(19) United States
(12) Patent Application Publication
(10) Pub. No.: US 2023/0404753 A1
(43) Pub. Date: Dec. 21, 2023

(54) DELIVERY SYSTEMS FOR REPLACEMENT HEART VALVES

(71) Applicant: EDWARDS LIFESCIENCES CORPORATION, Irvine, CA (US)

(72) Inventors: Hieu Minh Luong, Westminster, CA (US); Julio Cesar Sanchez, Garden Grove, CA (US); Alexander H. Cooper, Costa Mesa, CA (US); Jesse Robert Edwards, Silverado, CA (US); Hiroshi Okabe, Costa Mesa, CA (US); Karen Fromell Nesbitt, Newport Beach, CA (US); David Robert Landon, Huntington Beach, CA (US); Nikolai Brent Poulsen, Tustin, CA (US); Taylor Jacob Scheinblum, Newport Beach, CA (US); Eric Robert Dixon, Villa Park, CA (US); Cooper Ryan Rickerson, Huntington Beach, CA (US); Zachary Charles VanEvery, Irvine, CA (US)

(21) Appl. No.: 18/230,881

(22) Filed: Aug. 7, 2023

Related U.S. Application Data
(63) Continuation of application No. PCT/US2022/016150, filed on Feb. 11, 2022.
(60) Provisional application No. 63/273,402, filed on Oct. 29, 2021.
(60) Provisional application No. 63/148,501, filed on Feb. 11, 2021.

(51) Int. Cl. A61F 2/24

(52) U.S. Cl. CPC A61F 2/2436; A61F 2/2439

(57) ABSTRACT

A delivery system includes a suture attachment mechanism for maintaining a connection to a heart valve after initial deployment, thereby allowing recapture of the heart valve if desired. A plurality of suture portions is provided along a distal end portion of the delivery system, each suture portion having a first end fixed to the delivery system and a second end releasably coupled to a retention member on the delivery system. A knob on a handle of the delivery system is actuated for causing the second end of each suture portion to be released from its respective retention member, thereby allowing the suture portions to be decoupled from the heart valve. The suture portions are located only along a distal end portion of the delivery system for improving reliability and consistency.

### RELATED APPLICATIONS

This application is a continuation of International PCT Application No. PCT/US2022/016150, filed Feb. 11, 2022, published as WO/2022/174057, which claims the benefit of U.S. Provisional Application No. 63/148,501, filed Feb. 11, 2021; and U.S. Provisional Application No. 63/273,402, filed Oct. 29, 2021; the entirety of each of which is hereby incorporated by reference.

### FIELD

Certain embodiments disclosed herein relate generally to prostheses for implantation within a lumen or body cavity and delivery systems for a prosthesis. In particular, the prostheses and delivery systems relate in some embodiments to replacement heart valves, such as replacement mitral heart valves or replacement tricuspid heart valves.

### BACKGROUND

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments, such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. The ability to control the deployment of the prosthesis at the desired location can also be challenging.

### SUMMARY

Examples of the present disclosure are directed to a delivery system, such as but not limited to a delivery system for a replacement heart valve. Further examples are directed to methods of use to deliver and/or controllably deploy a prosthesis, such as but not limited to a replacement heart valve, to a desired location within the body. In some configurations, a replacement heart valve and methods for delivering a replacement heart valve to a native heart valve, such as a mitral valve, an aortic valve, or a tricuspid valve, are provided.

In some implementations, a delivery system and method are provided for delivering a replacement heart valve to a native mitral valve location. The delivery system and method may utilize a transseptal approach. In some implementations, components of the delivery system facilitate bending of a delivery device of the delivery system to steer a prosthesis from the septum to a location within the native mitral valve. In some implementations, a capsule is provided for containing the prosthesis for delivery to the native mitral valve location. The capsule may also be configured to recapture the prosthesis after initial deployment if another target implantation location is desired. In other implementations, the delivery system and method may be adapted for delivery of implants to locations other than the native mitral valve.

A suture-based release mechanism adapted for use with a delivery device for delivery of an implant (e.g., replacement heart valve or valve prosthesis) may include dual coaxial sliding shafts or subassemblies. The inner shaft may be a manifold to which sutures or tethers (e.g., ends of suture loops of a continuous suture or tether strand) are attached. The outer shaft may include one or more release windows that pushes the sutures or tethers (e.g., ends of suture loops) off the manifold for release.

The suture-based release mechanism may be incorporated into the delivery device. In other words, a delivery device may include a suture-based release mechanism involving dual coaxial sliding shafts or subassemblies that operate in conjunction to facilitate transition of the implant between a tethered configuration and an untethered (e.g., released) configuration upon actuation of an actuator (e.g., rotatable knob) of a proximal handle of the delivery device. The delivery device may include multiple suture or tether portions that are fixedly attached to a distal end portion of the delivery device at one end and inserted through an opening of an implant and then releasably coupled to retention members at the distal end portion of the delivery device. Thus, the suture or tether portions are only connected at a distal end portion of the delivery device and do not extend to the proximal handle of the delivery device. The actuator of the proximal handle may be configured to cause translation of one of the dual coaxial sliding shafts with respect to the other.

In some configurations, a delivery device for delivering an implant includes a shaft assembly comprising a proximal end portion and a distal end portion. The proximal end portion of the shaft assembly includes a handle including at least one actuator. The delivery device also includes at least one suture (e.g., a plurality of suture portions). A first end of the at least one suture (e.g., each of the plurality of suture portions) is permanently coupled to the distal end portion of the shaft assembly. A second end of the at least one suture (e.g., each of the plurality of suture portions) is removably coupled to at least one retention member (e.g., tab, finger, hook) of the distal end portion of the shaft assembly after being inserted through a coupling member (e.g., hole, eyelet) of an implant. In use, actuation of the at least one actuator causes the second end of the at least one suture (e.g., each of the plurality of suture portions) to be decoupled from the at least one retention member of the distal end portion of the shaft assembly.

The delivery device may include additional shafts, lumens or subassemblies to facilitate delivery of the implant to a desired implantation site (e.g., an outer sheath subassembly, a rail subassembly, a mid-shaft subassembly, and/or a nose cone subassembly). An outer sheath subassembly may be adapted to recapture the implant in-situ and then redeploy the implant at a new implantation site. A rail subassembly may facilitate bending of the delivery device to reach the desired implantation site. A mid-shaft subassembly may be adapted to retain a portion of the implant in a compressed configuration until the desired implantation site is reached and the implant is ready to deploy. The nose cone subassembly may facilitate access to the desired implantation site and guidance of the delivery device to the desired implantation site. The delivery device may include a handle with actuators (e.g., knobs) adapted to control movement (axial, bending, rotational movement) of the various subassemblies of the delivery device. The implant may be a prosthetic replacement heart valve and the desired implantation site may be within an annulus of a native heart valve (e.g., mitral valve, tricuspid valve, aortic valve).

In some implementations, the suture-based release mechanism includes an outer release shaft or subassembly having a proximal end and a distal end and an inner manifold shaft or subassembly having a proximal end and a distal end. The manifold shaft is coaxially positioned within the release shaft. The suture-based release mechanism may include a plurality of suture portions (which may be formed of a continuous piece of suture or tether wire) adapted to be removably tethered to an implant (e.g., inserted through an opening of or wrapped around a feature of a valve prosthesis, such as a replacement heart valve). The plurality of suture loops may be coupled to the manifold shaft. For example, a first end of each of the plurality of suture portions (e.g., loops) may be adapted to be removably coupled to at least one suture loop receiving member (e.g., tab, peg, finger) of the manifold shaft that is positioned proximal of the distal end (e.g., terminus) of the manifold shaft. A second end of each of the plurality of suture loops may be permanently (e.g., non-removably) coupled to the distal end of the manifold shaft. Relative sliding movement of the manifold shaft with respect to the release shaft from a locked configuration to an unlocked configuration causes release of the first end of each of the plurality of suture loops from the at least one suture loop receiving member, thereby allowing the first end of each of the plurality of suture loops to be untethered from the implant.

The relative sliding movement may include movement of the manifold shaft distally while the release shaft is stationary. The suture-based release mechanism (or delivery device comprising the release mechanism) may include a spring in the handle of the delivery device that is configured to keep the release mechanism in the locked configuration by default, wherein the spring exerts a distal spring force on the release shaft that must be overcome to transition the release mechanism to the unlocked configuration.

The at least one suture loop receiving member may comprise a plurality of tabs arranged circumferentially around the distal portion of the manifold shaft, wherein each of the plurality of tabs is adapted to receive a first end of at least one of the plurality of suture loops. A distal portion of the release shaft may include a plurality of windows, wherein each window of the plurality of windows is adapted to align with a respective one of the plurality of tabs of the manifold shaft. Sliding movement of the manifold shaft in a distal direction while keeping the release shaft fixed in position may cause a distal edge of each window of the release shaft to push the second end of each suture loop proximally along a respective one of the plurality of tabs of the manifold shaft until the second end of each suture loop is released from the respective one of the plurality of tabs, thereby allowing the implant (e.g., replacement heart valve) to be decoupled from the delivery device.

The at least one suture loop receiving member (e.g., tab, peg, finger) of the manifold shaft may reside within a respective opening or window proximal of the distal end of the manifold shaft. The second end of each of the plurality of suture loops may be permanently, or non-removably, coupled to a cog at the distal end of the manifold shaft including a plurality of tether cleats and then permanently glued or sealed between suture retention rings positioned on both sides of the tether cleats.

The plurality of suture loops may include three, four, five, six, seven, eight, nine, or more suture loops. The number of suture loops may correspond to the number of proximal eyelets (or other opening) located on a proximal end of the implant. During assembly, the first end of each of the plurality of suture loops may be inserted through a respective eyelet of the proximal end of the implant before being threaded through a release window of the release shaft and removably coupled to the at least one suture loop receiving member of the manifold shaft.

In one implementation with nine suture loops, the at least one suture loop receiving member (e.g., tab, peg, finger) of the manifold shaft or subassembly may comprise three tabs arranged circumferentially around the distal portion of the manifold shaft, wherein each of the three tabs is adapted to receive a first end of one or more of the plurality of suture loops. In this implementation, each tab receives three first ends of three suture loops. In this implementation, a distal portion of the release shaft may include three windows, wherein each window of the three windows is adapted to align with a respective one of the three tabs of the manifold shaft. In such an implementation, a second end of each of the nine suture loops may be non-removably coupled to a cog at the distal end of the manifold shaft. A portion of each of the nine suture loops may be looped through a respective eyelet positioned at a proximal end of the replacement heart valve. Sliding movement of the manifold shaft in a distal direction while keeping the release shaft fixed in position causes a distal edge of each window of the release shaft to push the second end of each of the nine suture loops proximally along a respective one of the three tabs of the manifold shaft until the second end of each of the nine suture loops is released from the respective one of the three tabs, thereby allowing the implant (e.g., replacement heart valve) to be decoupled from the delivery device.

The release shaft may include at least one radially inwardly-protruding retention member configured to be received within at least one slot of the manifold shaft so as to prevent rotation of the release shaft with respect to the manifold shaft to thereby maintain alignment of each window with a respective tab. Each of the plurality of tabs may have the substantially the same length or a different length.

In accordance with several implementations, a method of making or manufacturing a suture-based release mechanism to facilitate delivery of an implant includes permanently attaching a first end of a suture loop to a distal end of an inner tube, threading a free second end of the suture loop through a hole of the implant, inserting the free second end of the suture loop through a window positioned along a distal end portion of an outer tube coaxially surrounding the inner tube, placing the free second end of the suture loop onto a tab positioned along a distal end portion of the inner tube to removably couple the free second end of the suture loop to the tab, and causing the distal end of the outer tube to be advanced distally to align with the distal end of the inner tube such that the second end of the suture loop is prevented from coming off of the tab until the implant is in a desired position for implantation.

In accordance with several implementations, a method of making a suture-based release mechanism to facilitate delivery of an implant includes permanently attaching a first end of a suture loop to a distal end of an inner tube, threading a loop end of the suture loop through a hole of the implant, inserting the loop end of the suture loop through a slot positioned along a proximal tether retention component at a distal portion of the inner tube to removably couple the loop end of the suture loop to the proximal tether retention component, and inserting a free end of a release suture through the loop end of the suture loop to secure the suture loop to the inner tube.

The process described above may be repeated for multiple suture loops formed from a single continuous suture or tether strand. The distal end of the inner tube may comprise multiple tether cleats spaced apart circumferentially. These tether cleats may form a plurality of proximal members that the single continuous suture or tether strand is wrapped around to form a plurality of proximal suture loop ends. An assembly member may include a plurality of circumferentially-spaced apart pegs or cleats to form a plurality of distal members that the single continuous suture or tether strand is wrapped around to form a plurality of distal suture loop ends. The words “suture” and “tether” may be used interchangeably herein.

The proximal suture loop ends and the distal suture loop ends may be circumferentially offset from each other such that each strand portion connects a proximal suture loop end to a circumferentially offset distal suture loop end in an alternating serpentine fashion. For example, a strand is wrapped around a first proximal member to form a first proximal suture loop end and then brought back down to a first distal member that may be spaced apart (or offset circumferentially) from the first proximal member and wrapped around the first distal member to form a second suture loop end (a first distal suture loop end) and then brought back up to a second proximal member spaced apart (or offset circumferentially) from the first distal member to form a third suture loop end (a second proximal suture loop end) in a serpentine fashion. This process is repeated until the desired number of suture loop ends have been created. The two ends of the single continuous suture or tether strand may be knotted together (and optionally glued or otherwise adhered together) after forming the multiple suture loops and coupling them to eyelets or other retention members on a proximal end of the implant.

In accordance with several implementations, a method of facilitating delivery of an implant within a body of a patient using a suture-based release mechanism includes advancing a distal end portion of a delivery device to a desired implantation location. The delivery device includes dual, coaxial sliding shafts (e.g., an inner shaft and an outer shaft). At least one suture loop is pre-attached to the implant during manufacture of the delivery device and a first end of the suture loop is non-removably coupled to a distal end of an inner shaft of the dual, coaxial sliding shafts during manufacture of the delivery device. A second end of suture loop is removably coupled to a suture retention member of the manifold after having been inserted through a retention member (e.g., eyelet) of the implant. A distal end portion of an outer shaft of the two shafts includes a release window adapted to push the second end of the suture loop off of the suture retention member upon relative sliding movement of the inner shaft with respect to the outer shaft. The method also includes advancing the inner shaft distally with respect to the outer shaft so as to cause decoupling of the second end of the suture loop from the suture retention member and out of the release window and withdrawing the shafts to allow the second end of the suture loop to be decoupled from the retention member of the implant, thereby allowing the implant to remain in the desired implantation location when the delivery system is removed from the patient.

In some implementations, a loop end of the suture loop is inserted through a slot of a proximal tether retention component of the inner shaft after having been inserted through a retention member (e.g., proximal-most eyelet of an inflow strut of a frame) of the implant. A release suture may be inserted through the loop end of the suture loop after the loop end of the suture loop is inserted through the slot. The method may also include advancing the inner shaft distally with respect to the outer shaft, withdrawing the release suture from the loop end of the suture loop, and decoupling the loop end of the suture loop from the retention member of the implant, thereby allowing the implant to remain in the desired implantation location when the delivery device is removed from the patient.

During implant delivery, the outer release shaft or subassembly may be kept in a distal position by a spring in the handle at the proximal end of the delivery device, securing the suture loop(s) to the inner manifold shaft or subassembly. When the user advances a manifold/release knob of the handle, the outer release shaft moves forward with the inner manifold shaft via the biased compression spring force of the spring until a release shaft handle adapter hits a hard stop member in the handle. Continued advancement of the inner manifold shaft extends the inner manifold shaft distally while the outer release shaft stays in place due to contact with the hard stop member in the handle. The distal edge of a release shaft window abuts the suture loop end and pushes it proximally, releasing it from a suture receiving member (e.g., tab, finger, peg) on the underlying inner manifold shaft. Retraction of the release and manifold shafts (e.g., by rotating the manifold/release knob proximally) unthreads or uncouples the suture loops from the valve eyelets. The suture loops are removed from the body with the delivery system. The suture loops may be formed from a single continuous tether strand in which the two ends of the continuous tether strand are knotted and glued together after forming the suture loops.

In accordance with several configurations, a valve prosthesis adapted for non-uniform compression during loading into a capsule includes a self-expanding frame configured to transition between a compressed configuration and an expanded configuration. The frame includes at least one row of cells forming a ring. The valve prosthesis also includes a plurality of prosthetic valve leaflets coupled to the frame. The frame includes a plurality of pre-curved axial connection portions, each axial connection portion extending between a top end and bottom end of each cell of the at least one row of cells. Each axial connecting portion is adapted to bend in a predetermined manner for accommodating changes in cell height during non-uniform compression of the valve prosthesis.

In accordance with several configurations, a valve prosthesis includes a self-expandable frame configured to transition between a compressed configuration and an expanded configuration. The frame includes a plurality of rows of cells formed by struts, wherein the cells form a chevron-shaped cell structure. At least one cell of a distal-most row of the plurality of rows of cells includes an axial strut connecting a distal apex of the cell with a distal apex of a bordering cell in a row immediately above the distal-most row. The axial strut includes a bow-spring structure adapted to prevent cell ovality during the transition between the compressed configuration and the expanded configuration, and vice-versa.

The bow-spring structure may include a dual bow-spring structure in which the axial strut comprises two axial strut segments connected at their proximal and distal ends but separated along their lengths. Each of the cells of the distal-most row may include an axial strut connecting a distal apex of the respective cell with a distal apex of a respective bordering cell in a row immediately above the distal-most row. Each of the axial struts of the cells of the distal-most row comprises a bow-spring structure adapted to prevent cell ovality during the transition between the compressed configuration and the expanded configuration, and vice-versa. The bow-spring structures may be asymmetric or symmetric.

In accordance with several configurations, a dual-frame valve prosthesis includes an inner frame including an inflow portion having an inflow end, an outflow portion having an outflow end, and an intermediate portion extending between the inflow portion and the outflow portion. The inflow end of the inner frame includes a plurality of inflow struts (e.g., axial proximal struts or beams) including a plurality of eyelets (e.g., two, three or more eyelets). The outflow end of the inner frame includes a plurality of anchors (e.g., distal anchors or ventricular anchors). The valve prosthesis also includes an outer frame including an inflow portion having an inflow end, an outflow portion including an outflow end, and an intermediate portion extending between the inflow portion and the outflow portion. The inflow end of the outer frame includes a plurality of inflow struts (e.g., axial proximal struts or beams) including a plurality of eyelets. At least one of the plurality of eyelets of each of the plurality of inflow struts of the outer frame is configured to engage with at least one of the plurality of eyelets of the plurality of inflow struts of the inner frame.

The valve prosthesis may also include a skirt assembly positioned between the inner frame and the outer frame. The skirt assembly includes an integral piece of cloth material with varying diameters, the integral piece of cloth material including a body portion, a plurality of proximal extensions extending from the body portion, and a plurality of distal extensions extending from the body portion. In some configurations, the plurality of proximal extensions is positioned between the inflow portion of the inner frame and the inflow portion of the outer frame. The body portion of the skirt assembly may be positioned external to the intermediate portion of the outer frame. The plurality of distal extensions may be positioned between the outflow portion of the inner frame and the outflow portion of the outer frame.

In some implementations, one or more of the plurality of proximal extensions include a tab configured to be positioned between one or more of the plurality of inflow struts of the inner frame and one or more of the plurality of inflow struts of the outer frame. In some implementations, one or more of the plurality of distal extensions include a hole configured to allow blood to flow into a volume between the inner frame and the outer frame.

In some implementations, the plurality of proximal extensions and/or the plurality of distal extensions comprise a trapezoidal shape. In some implementations, the plurality of proximal extensions is sewn together via one or more sutures when the valve prosthesis is assembled. In some implementations, the plurality of distal extensions is sewn together via one or more sutures when the valve prosthesis is assembled.

The one or more sutures may include at least one interlock stitch instead of a knot. At least one edge of the cloth material of the skirt assembly may be melted (e.g., using laser or soldering iron) to create a smooth edge surface. In some implementations, a valve assembly is positioned within the inner frame, the valve assembly including a plurality of prosthetic leaflets, wherein a cusp of each of the plurality of prosthetic leaflets is sutured to the skirt assembly using two different stitch lines (e.g., double stitch line).

In some configurations, the inflow struts of the outer frame each include a bendable tab that is unattached to the inflow strut of the outer frame along at least a portion of the bendable tab such that the bendable tab can bend along an independent plane from the respective inflow strut of the outer frame. The bendable tab may include at least one eyelet that is configured to engage with at least one of the plurality of eyelets of the plurality of axial inflow struts of the inner frame.

In some implementations, the inflow end of the outer frame and the inflow end of the inner frame are mechanically attached together via a dovetail joint configuration or a “puzzle piece” fit configuration.

In some implementations, the inflow struts of the inflow end of the outer frame and the inflow struts of the inflow end of the inner frame are attached together and proximal-most ends of at least two of the axial inflow struts are configured to be positioned at an offset distance from each other (e.g., staggered heights). Each adjacent inflow strut may be offset or they may be offset in pairs or other numbered groups.

In some implementations, at least some of the plurality of anchors include an attachable anchor dampener that does not comprise foam. The attachable anchor dampener may be configured to have a first portion configured to engage a native heart valve leaflet. The first portion may be more rigid than a second portion configured to contact a septal wall or annulus of a heart. The second portion may be configured to provide a cushioned contact surface.

In some implementations, at least some of the plurality of anchors include a metallic cushion anchor tip configured to distribute and dampen a load exerted on native tissue in contact with the anchor tip. The metallic cushion anchor tip may include a nitinol material. In one configuration, the metallic cushion anchor tip is a whisk configuration formed from a plurality of wire hoops.

In some implementations, at least some of the plurality of anchors include an anchor tip that is configured to provide a cushioning effect in a radially outward direction to reduce a likelihood of conduction disturbances caused by the anchor in contact with a septal wall of a heart and to provide rigidity in a radially inward direction to facilitate capture of native heart valve leaflets.

In accordance with several configurations s, a dual-frame valve prosthesis comprising co-organizing features to facilitate alignment and registration during compression and expansion of the dual frames of the dual-frame valve prosthesis includes an inner frame and an outer frame comprising one or more co-organizing features (e.g., a hammer-head proximal eyelet design and/or the distal apexes of the inner frame and the outer frame are circumferentially offset).

### BRIEF DESCRIPTION OF THE DRAWINGS

FIG. 1 shows an embodiment of a delivery system for an implant, such as a dual-frame heart valve prosthesis.

FIG. 2 shows a perspective view of a dual-frame valve prosthesis that may be delivered using the delivery system described herein.

FIG. 2A shows a side view of an inner frame of the dual-frame valve prosthesis of FIG. 2.

FIG. 2B shows a side view of an outer frame of the dual-frame valve prosthesis of FIG. 2.

FIG. 2C shows a side perspective view of a fully-assembled dual-frame valve prosthesis including a skirt assembly and padding.

FIGS. 2D-1 to 2D-3 illustrate how structural instability (e.g., strut buckling) can occur during compression of a standard chevron-cell frame structure.

FIGS. 2E-1 to 2E-4 illustrate various views of an embodiment of an inner frame having asymmetric “bow spring” structural mechanisms in compressed, partially-compressed, and expanded configurations.

FIGS. 2F-1 and 2F-2 illustrate embodiments of inner frames having highly asymmetric “bow spring” structural mechanisms and minimally asymmetric “bow spring” structural mechanisms, respectively.

FIGS. 2G-1, 2G-2, and 2G-3 show various views of an embodiment of an inner frame having symmetric “bow spring” structural mechanisms.

FIGS. 2G-4A, 2G-4B, 2G-5, 2G-6, 2G-7, 2G-8, 2G-9A, 2G-9B, 2G-10, 2G-11A, 2G-11B, 2G-11C, 2G-12, 2G-13, 2G-14, 2G-15, 2G-16, and 2G-17 illustrate various views of embodiments of anchor tips of a frame of a replacement heart valve, such as an inner frame of a dual-frame valve prosthesis.

FIGS. 2G-18A, 2G-18B, 2G-19, 2G-20A, 2G-20B, 2G-21A, 2G-21B, 2G-22, 2G-23A, 2G-23B, 2G-24A, 2G-24B, 2G-25A, 2G-25B, 2G-26A, 2G-26B, 2G-26C, 2G-27A and 2G-27B illustrate various views of embodiments of an anchor tip of a frame of a replacement heart valve, such as an inner frame of a dual-frame valve prosthesis.

FIG. 2H shows a side view of an embodiment of an outer frame including co-organizing frame features to facilitate improved operation with the inner frames described herein throughout transitory loading and deployment configurations.

FIGS. 2I-1 to 2I-3 illustrate various eyelet designs configured to reduce rotational and/or translational movement between an outer frame and inner frame of a dual-frame valve prosthesis.

FIG. 2J-1 illustrates an outer frame without certain co-organizing frame features. FIG. 2J-2 illustrates an outer frame having a co-organizing feature designed to straddle an inner frame axial strut to facilitate alignment.

FIGS. 2J-3 and 2J-4 illustrate another embodiment of a frame of a heart valve prosthesis where heights of proximal-most struts (e.g., tether attachment struts) of the frame are alternately varying or are offset.

FIG. 2K-1 illustrates how an outer frame can adversely interact with an anchor on an inner frame of a dual-frame valve prosthesis during crimping. FIG. 2K-2 shows how an implementation of an outer frame can be designed such that distal outflow portions of the outer frame avoid interaction with inner frame anchors during crimping.

FIGS. 2L-1 to 2L-3 illustrates various implementations of an outer frame design of a dual-frame valve prosthesis showing various options of a connection or attachment structure between the proximal eyelets and the connecting struts of the outer frame.

FIGS. 2L-4 to 2L-6 illustrate various embodiments of tabs and/or eyelets of a frame, such as an outer frame of a dual-frame valve prosthesis.

FIGS. 2M-1 and 2M-2 illustrate various implementations of a dual-frame valve prosthesis having various radii of curvature profiles when an inner frame and an outer frame are engaged.

FIGS. 2N-1 and 2N-2 illustrate one example of an outer frame. FIGS. 2N-3 and 2N-4 illustrate another example of an outer frame.

FIG. 20-1 illustrates a dual-frame valve prosthesis in which an inner frame and an outer frame are engaged in a pre-expansion state where the outer frame is not deployed. FIG. 20-2 illustrates a dual-frame valve prosthesis in which an inner frame and an outer frame are engaged in a capsule retracted state where the outer frame is deployed.

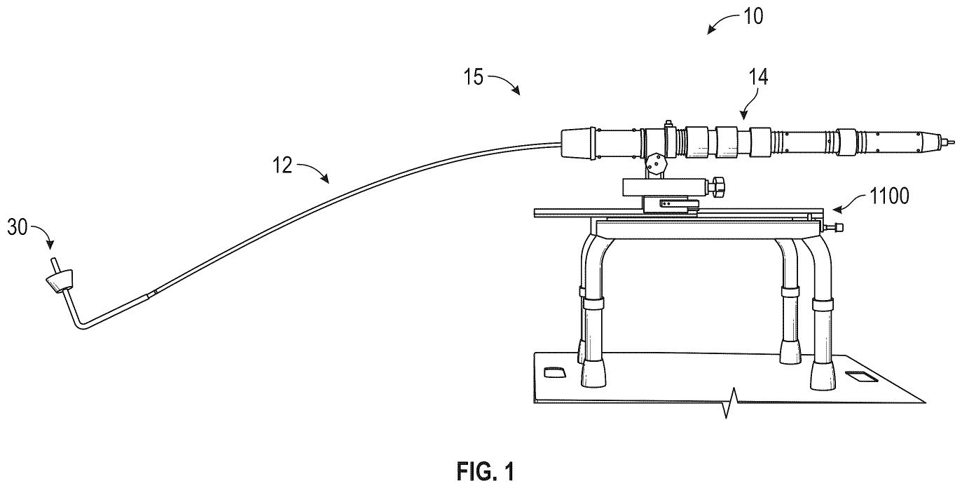

FIGS. 2P-1 to 2P-7 illustrate various embodiments of engaging an inner frame and an outer frame for forming the dual-frame heart valve prosthesis.

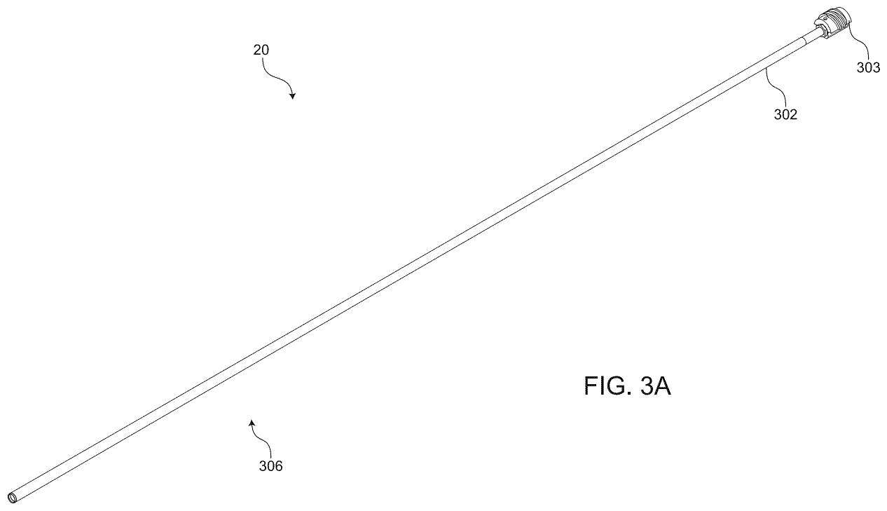

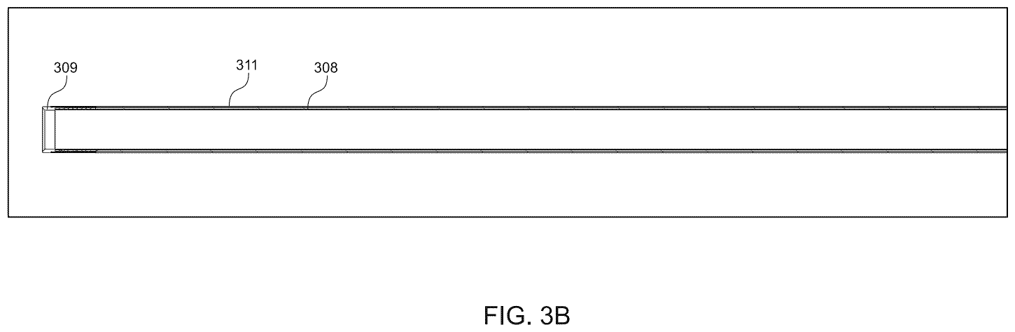

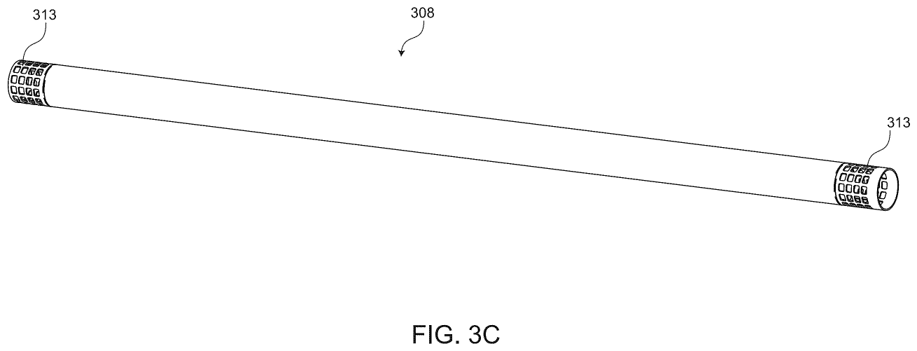

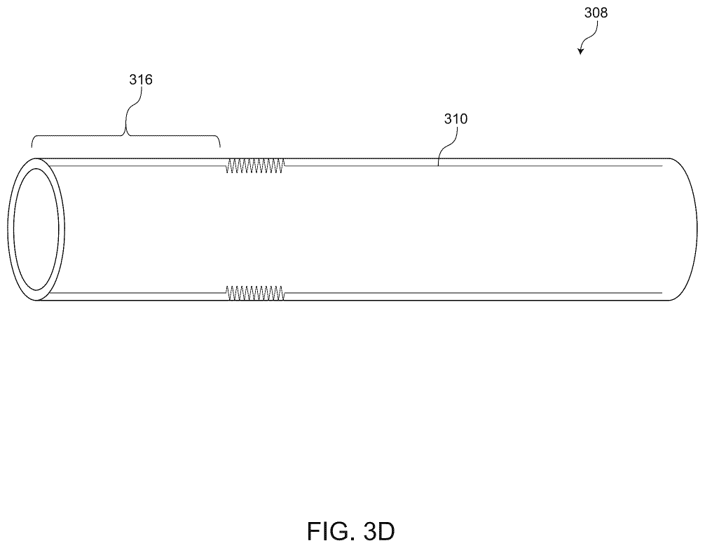

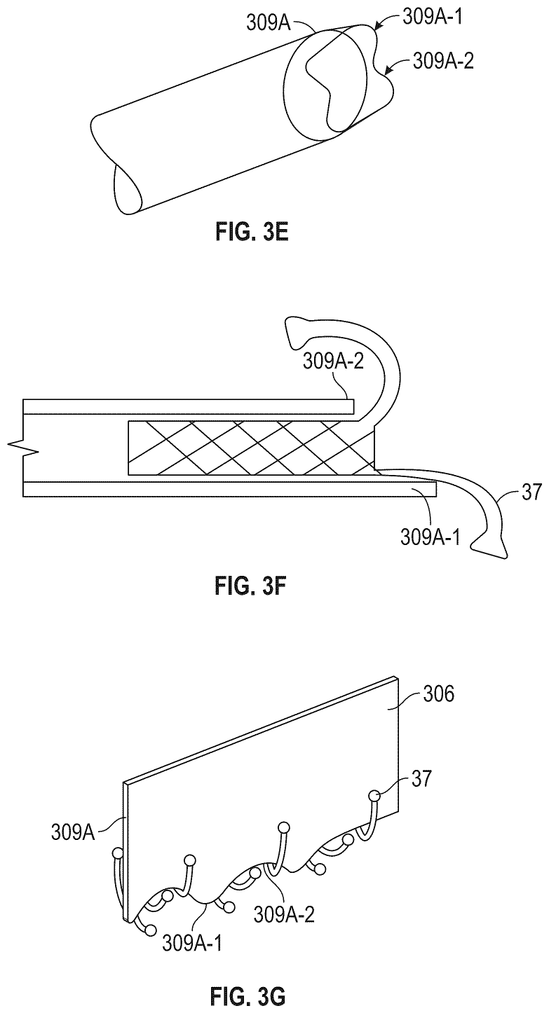

FIG. 3A shows a perspective view of an embodiment of an outer subassembly of a delivery device of the delivery system of FIG. 1. FIG. 3B illustrates a side-cross-section view of a capsule subassembly of the outer sheath subassembly of FIG. 3A. FIG. 3C shows a perspective view of a capsule stent, or distal hypotube, of the outer sheath subassembly of FIG. 3A. FIG. 3D shows how a portion of a liner extending along a length of the outer sheath subassembly can have built-in slack to facilitate flexible bending of the outer subassembly.

FIGS. 3E to 3G illustrate another embodiment of a distal capsule tip of a capsule subassembly.

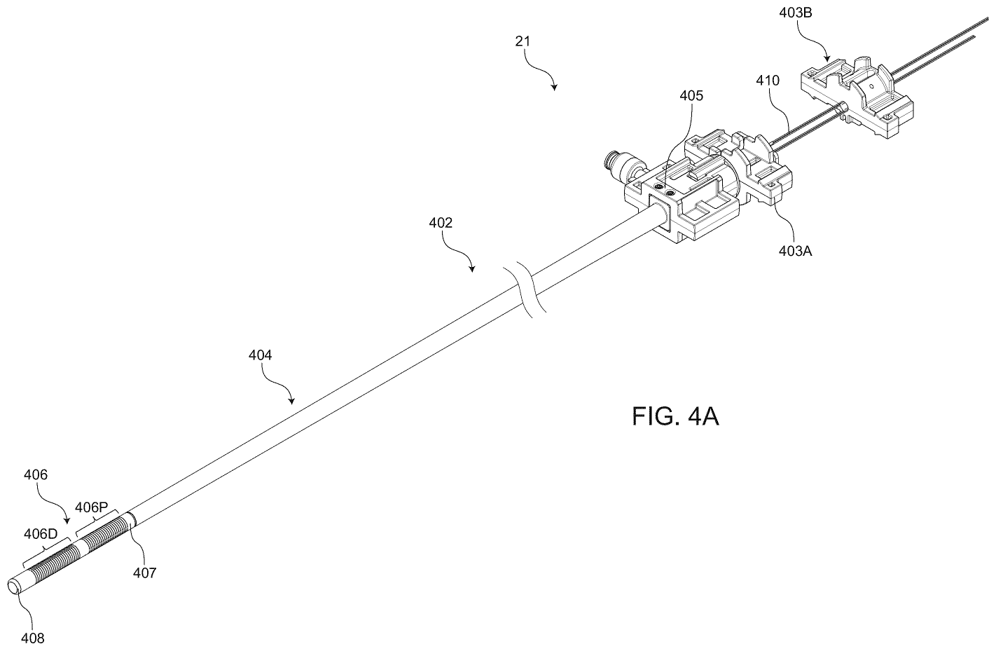

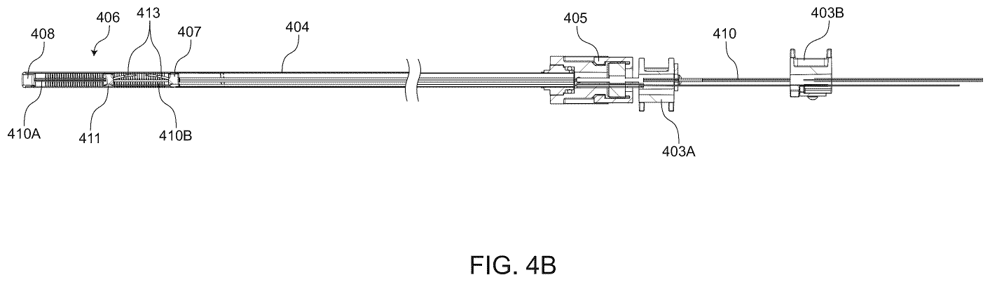

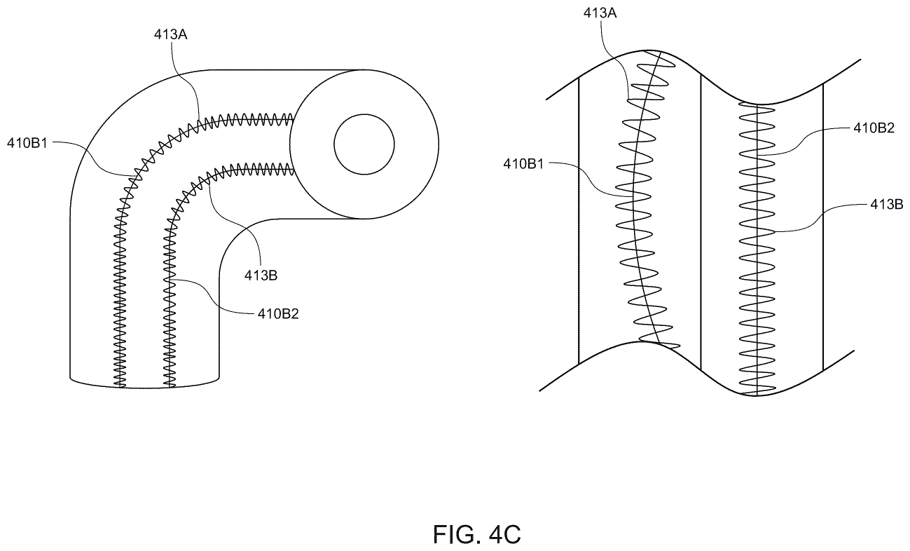

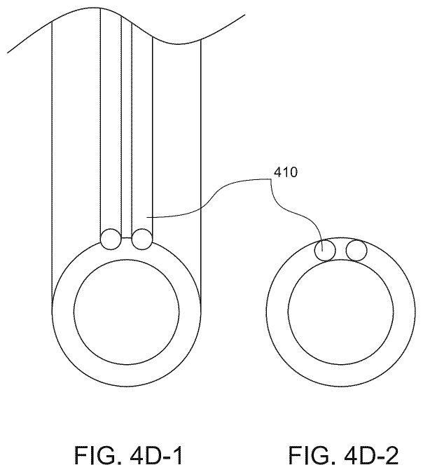

FIG. 4A shows a perspective view of a rail subassembly of the delivery device of the delivery system of FIG. 1. FIG. 4B shows a side cross-section view of the rail subassembly of FIG. 4A. FIG. 4C schematically illustrates how an outer compression coil and pull wire can have a longer length than an inner compression coil and pull wire of the rail subassembly. FIGS. 4D-1 and 4D-2 schematically illustrates thru-wall welding techniques performed during manufacture of the rail subassembly (as compared to prior direct welding techniques).

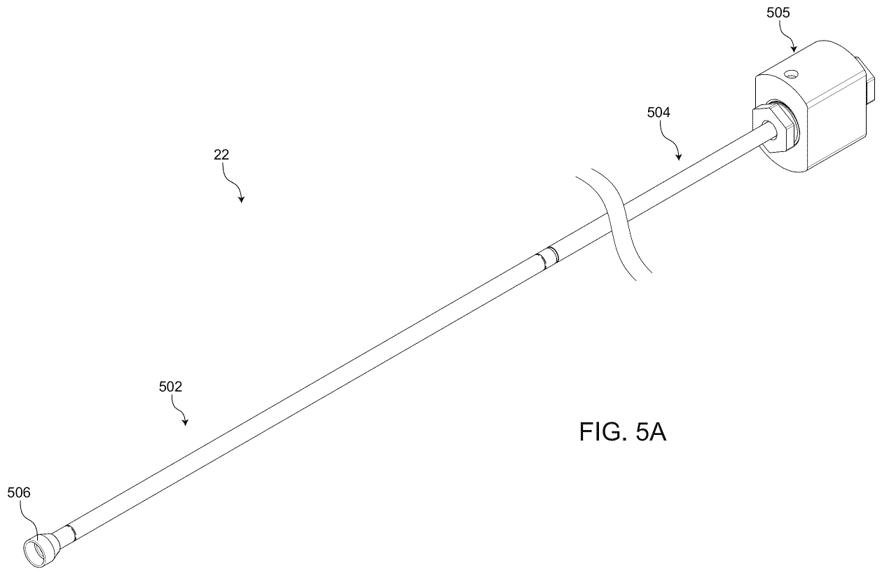

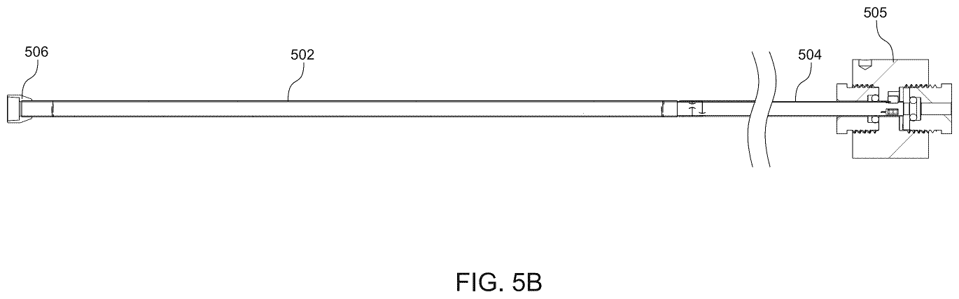

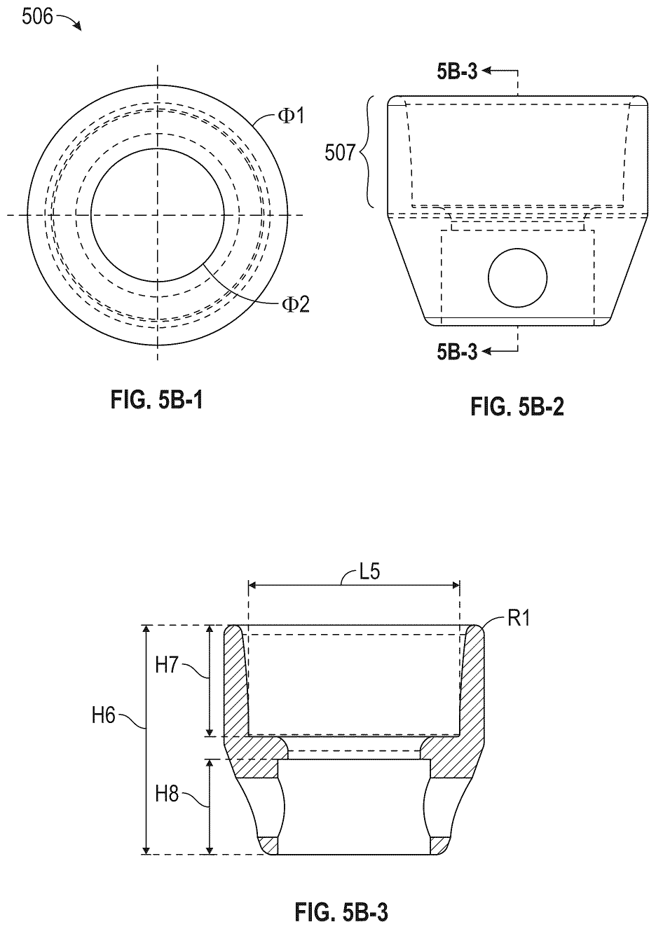

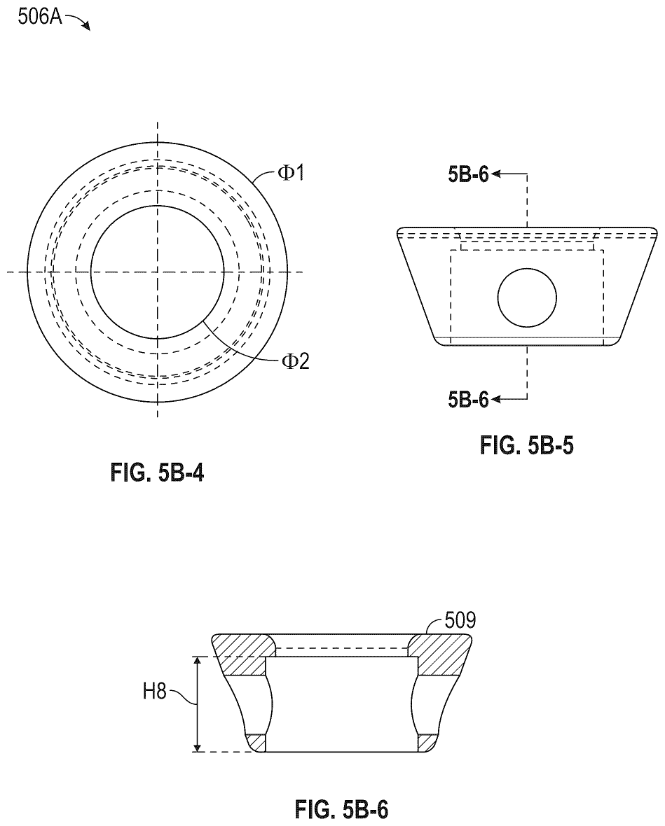

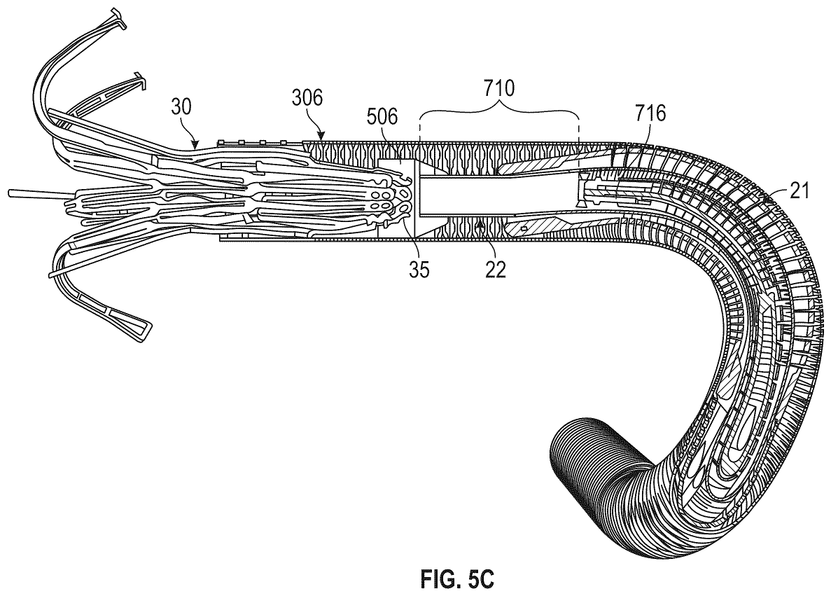

FIG. 5A shows a perspective view of a mid-shaft subassembly of the delivery device of the delivery system of FIG. 1. FIG. 5B illustrates a side cross-section view of the mid-shaft subassembly of FIG. 5A.

FIGS. 5B-1 to 5B-3 illustrate an embodiment of a distal end of the mid-shaft subassembly. FIGS. 5B-4 to 5B-6 illustrate another embodiment of a distal end of the mid-shaft subassembly. FIG. 5C illustrates a side cross-section view of a distal end portion of the shaft assembly, including the mid-shaft subassembly.

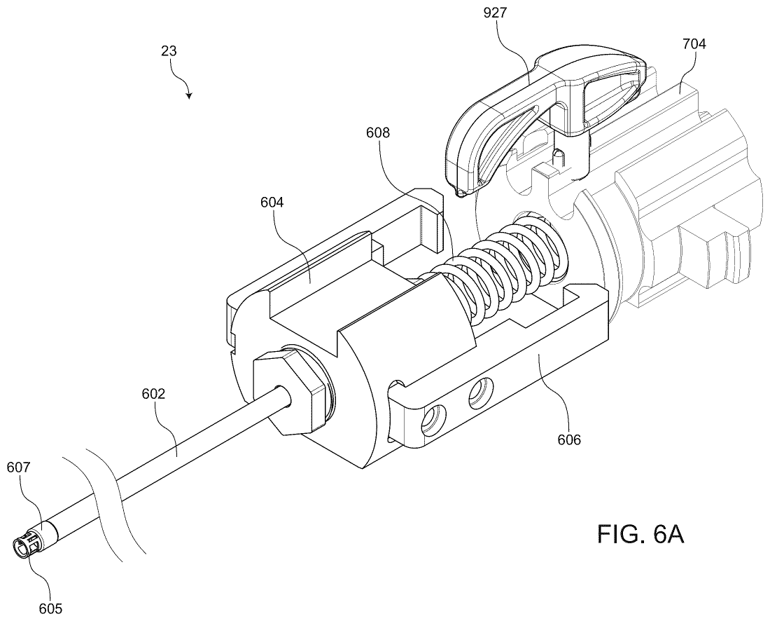

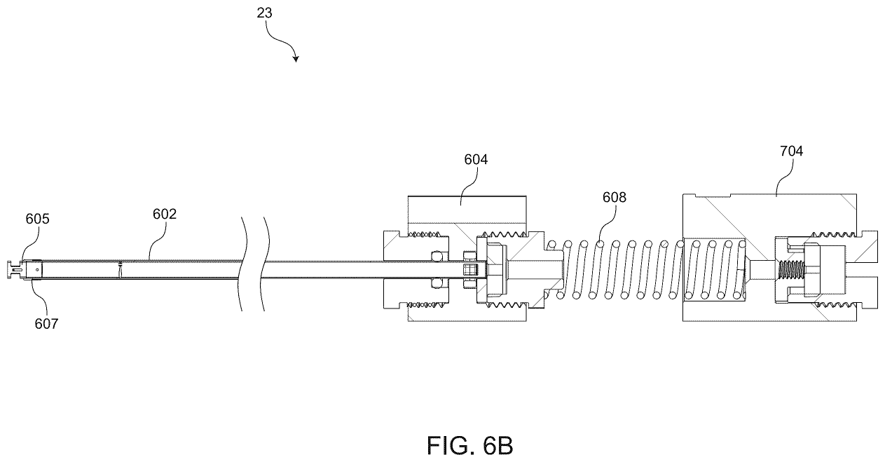

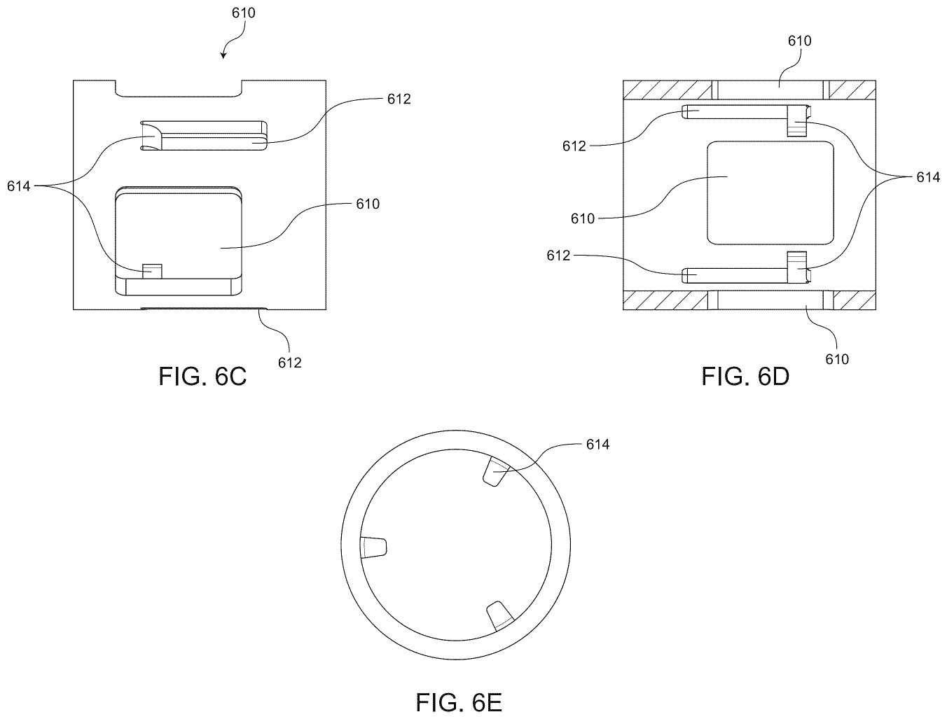

FIG. 6A shows a perspective view of a release subassembly of the delivery device of the delivery system of FIG. 1. FIG. 6B shows a side cross-section view of the release subassembly of FIG. 6A. FIG. 6C shows a close-up side view of a distal end portion of the release subassembly. FIG. 6D shows a side cross-section view of the distal end portion of the release subassembly. FIG. 6E shows a bottom view of the distal end of the release subassembly.

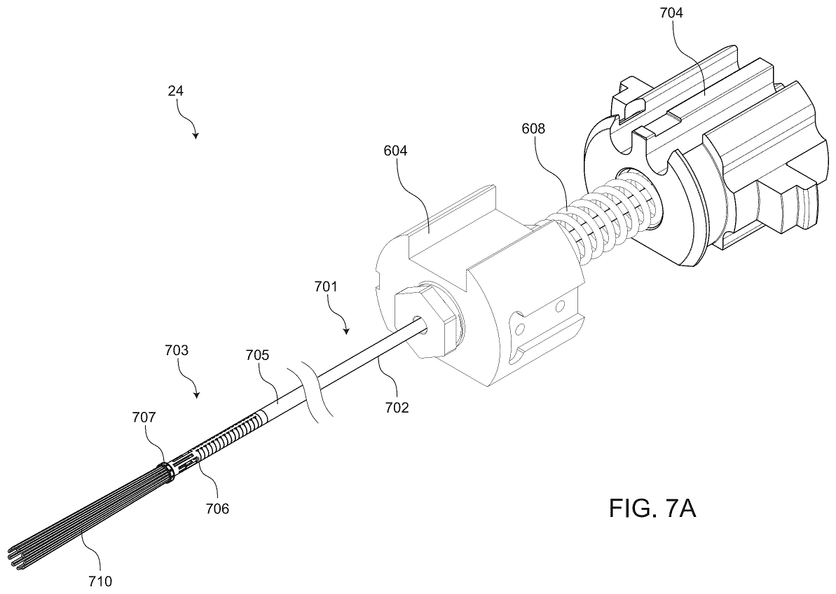

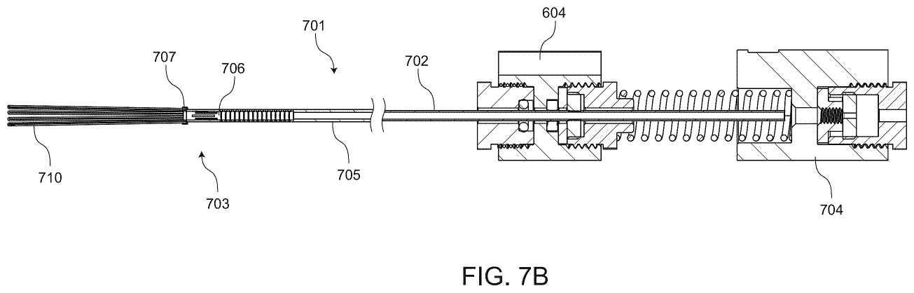

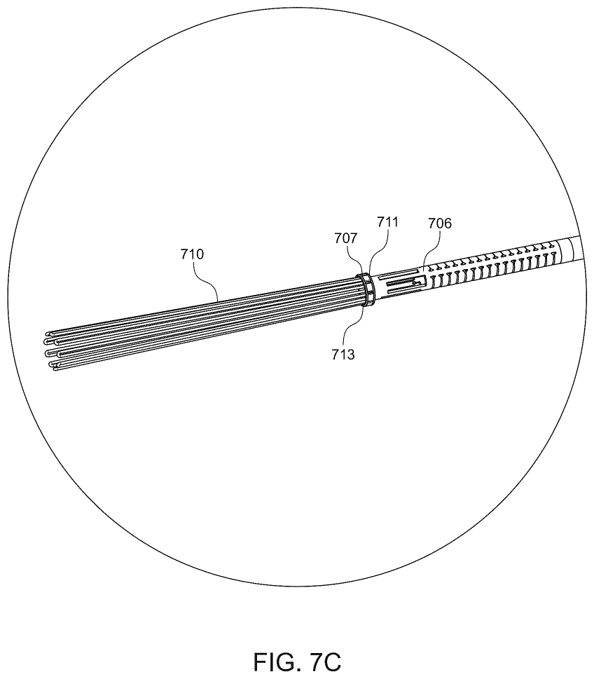

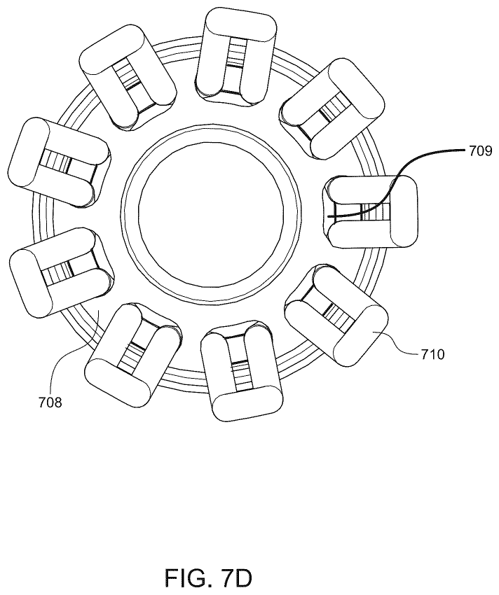

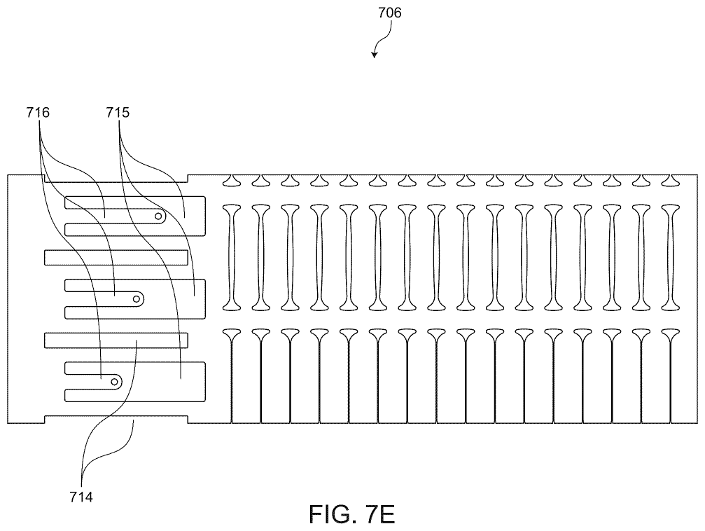

FIG. 7A shows a perspective view of a manifold subassembly of the delivery device of the delivery system of FIG. 1. FIG. 7B shows a side cross-section view of the manifold subassembly of FIG. 7A. FIG. 7C shows a close-up view of a distal end portion of the manifold subassembly. FIG. 7D shows a bottom view of the distal end portion of the manifold subassembly. FIG. 7E shows a flat cut pattern of a distal end portion of the manifold subassembly.

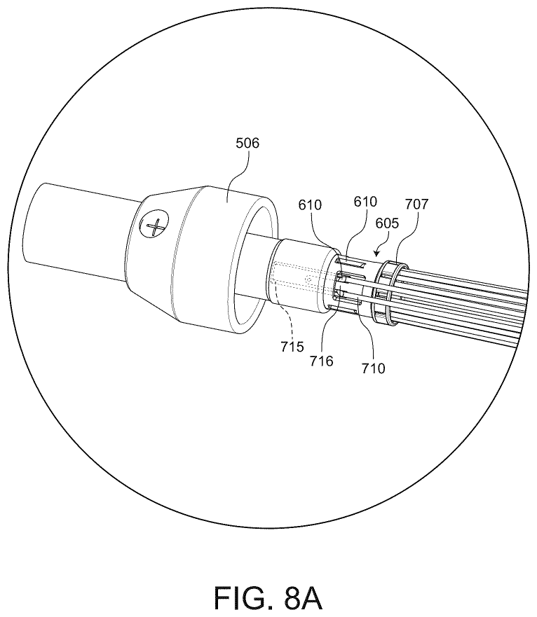

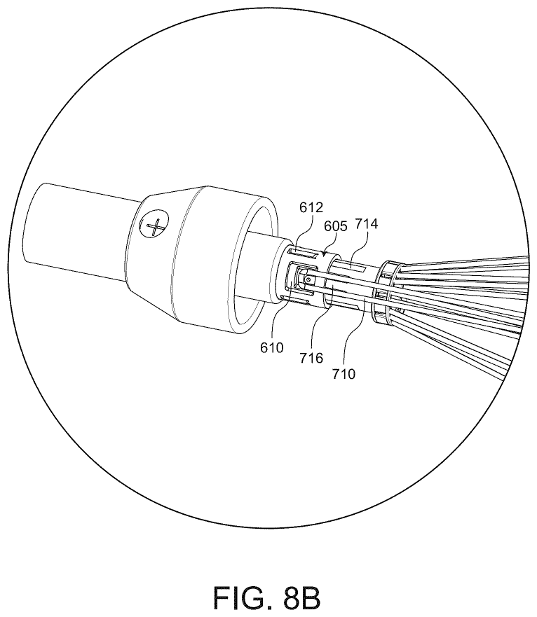

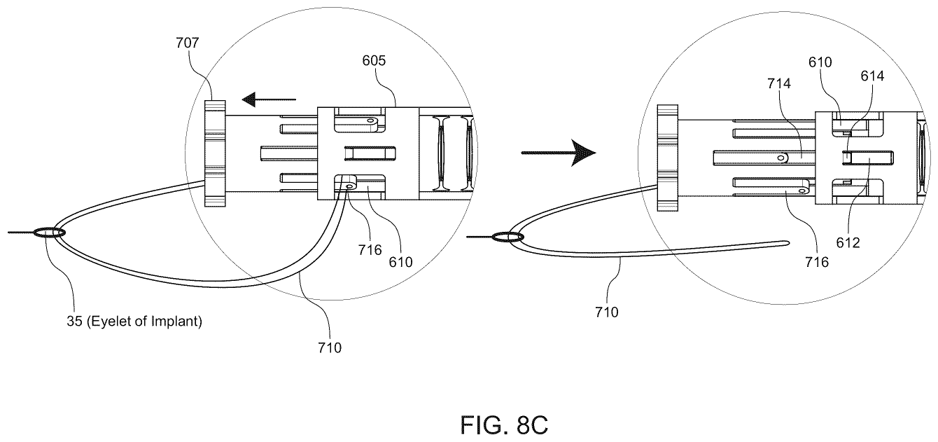

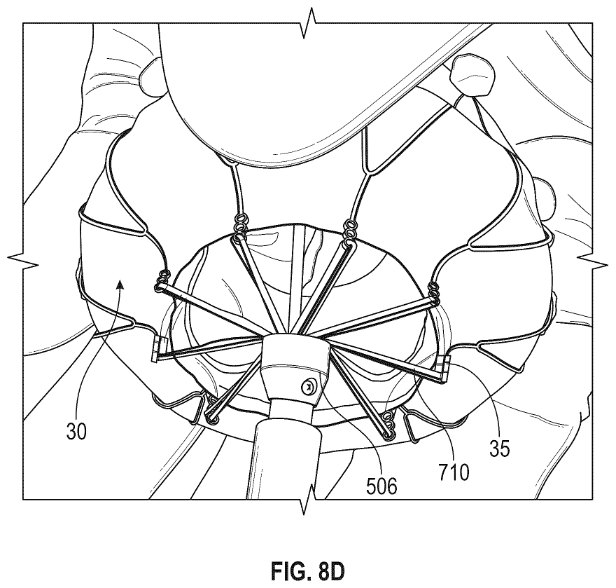

FIGS. 8A and 8B show distal end portions of the release and manifold assemblies in a locked configuration and unlocked configuration, respectively.

FIG. 8C illustrates tethering and untethering of a suture using the release and manifold assemblies. FIG. 8D shows suture loops tethered to the eyelets of the valve prosthesis while also tethered to the manifold subassembly of the delivery device.

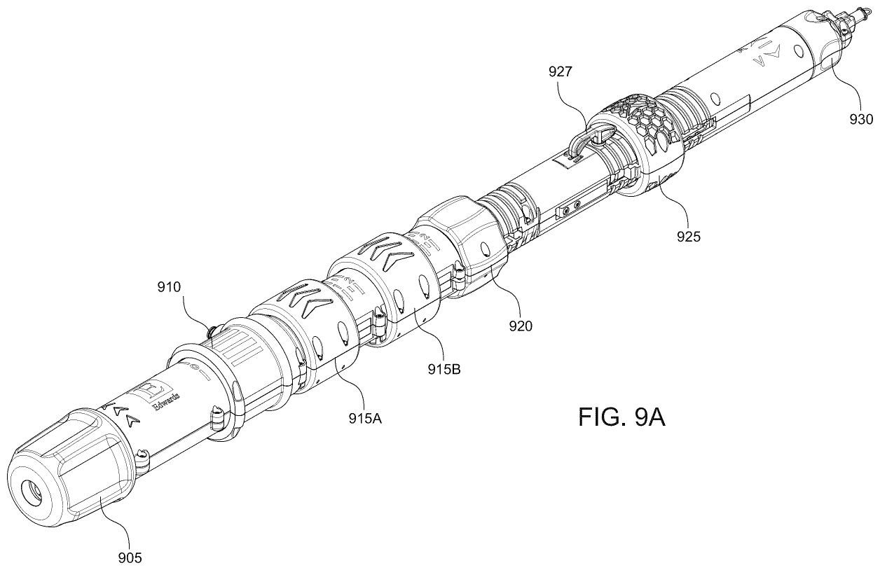

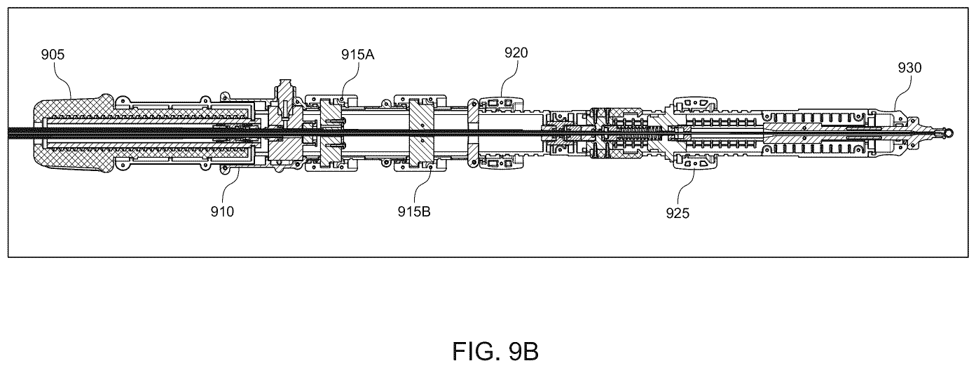

FIG. 9A shows a perspective view of a handle of the delivery device of FIG. 1. FIG. 9B shows a side cross-section view of the handle of the delivery device.

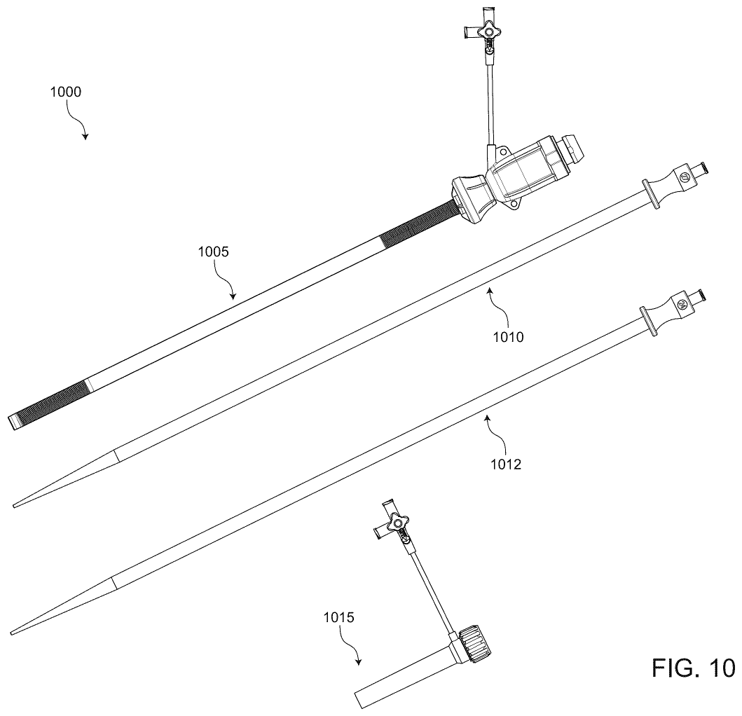

FIG. 10 shows components of an introducer assembly of the delivery system of FIG. 1.

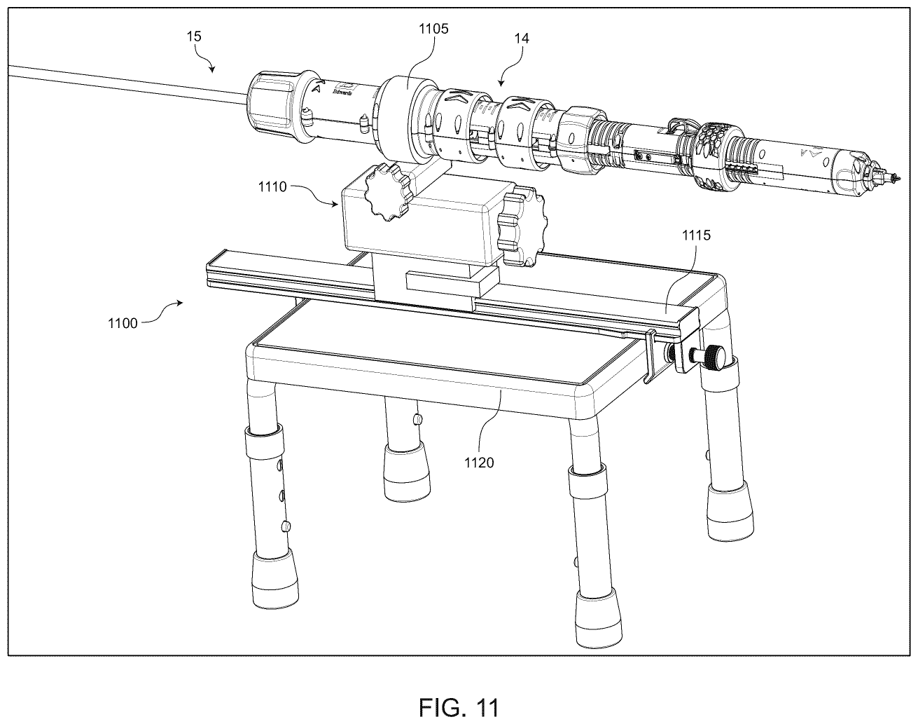

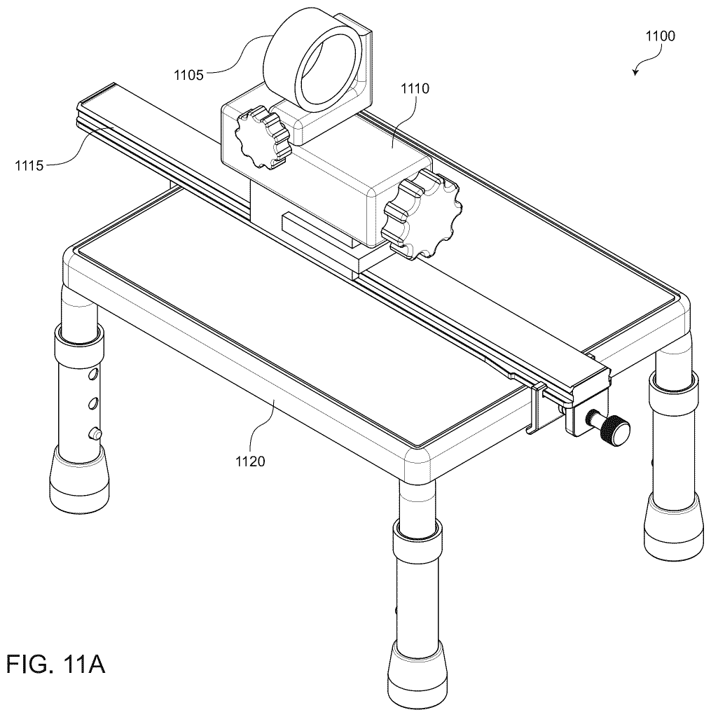

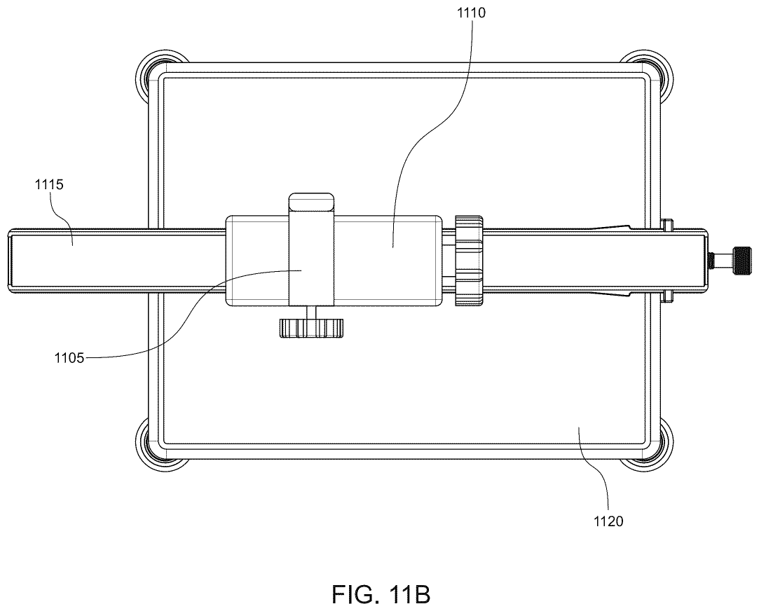

FIG. 11 illustrates how the handle of the delivery device interfaces with an embodiment of a stabilizer assembly of the delivery system of FIG. 1. FIG. 11A shows a perspective view of the stabilizer assembly without the delivery device attached. FIG. 11B shows a top view of the stabilizer assembly of FIG. 11A.

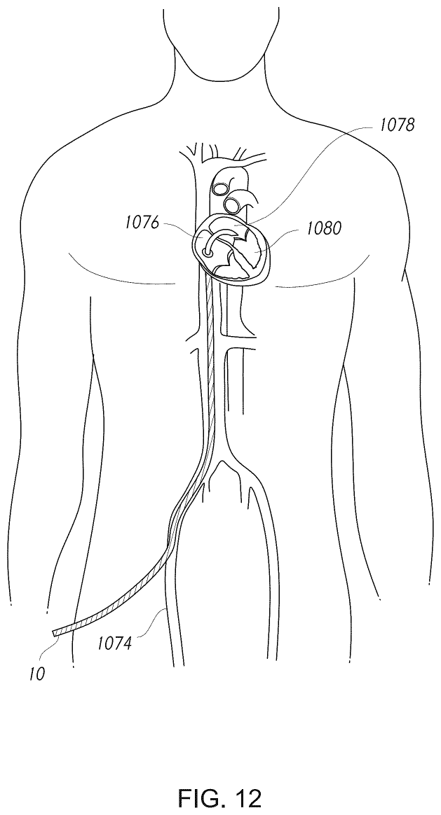

FIG. 12 illustrates a schematic representation of a transfemoral and transseptal delivery approach.

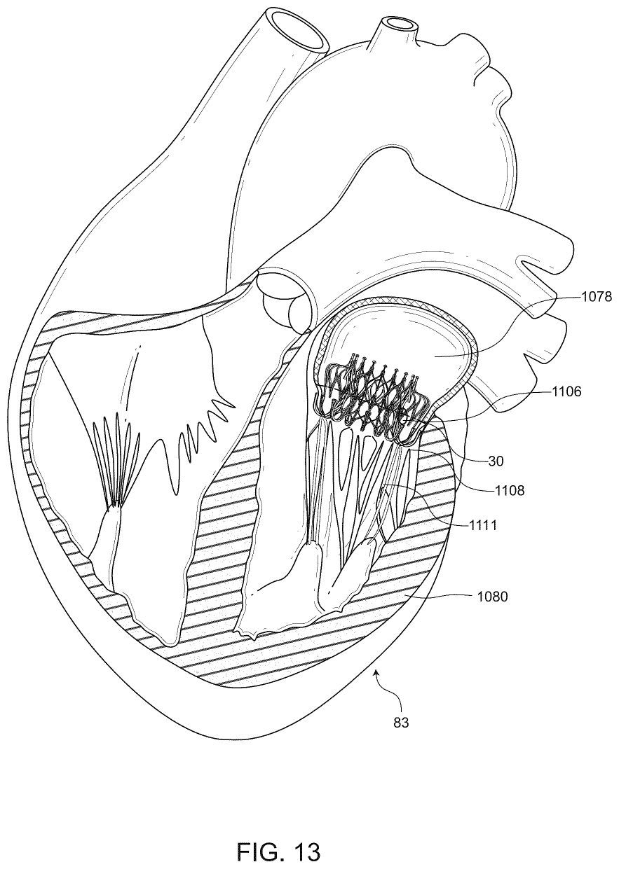

FIG. 13 illustrates a schematic representation of a valve prosthesis positioned within a native mitral valve (shown without a skirt assembly to facilitate visualization of interface with native heart valve structures).

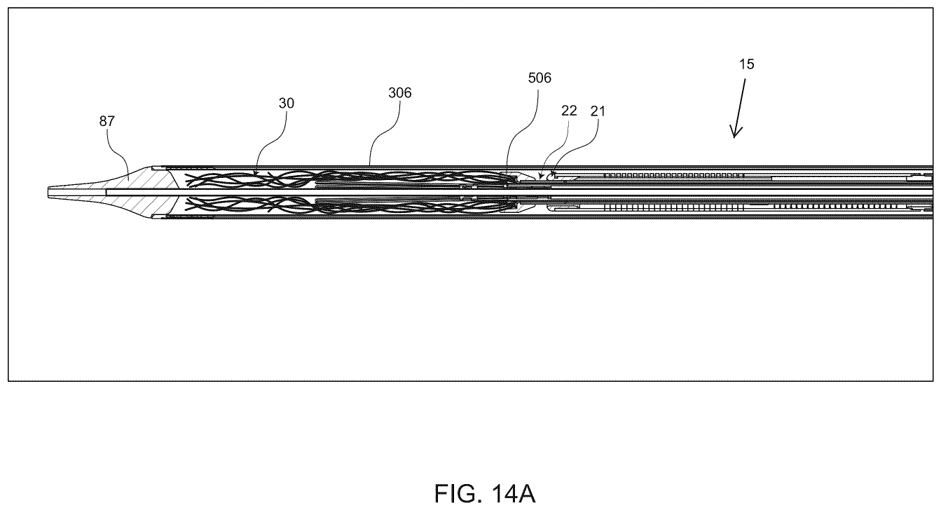

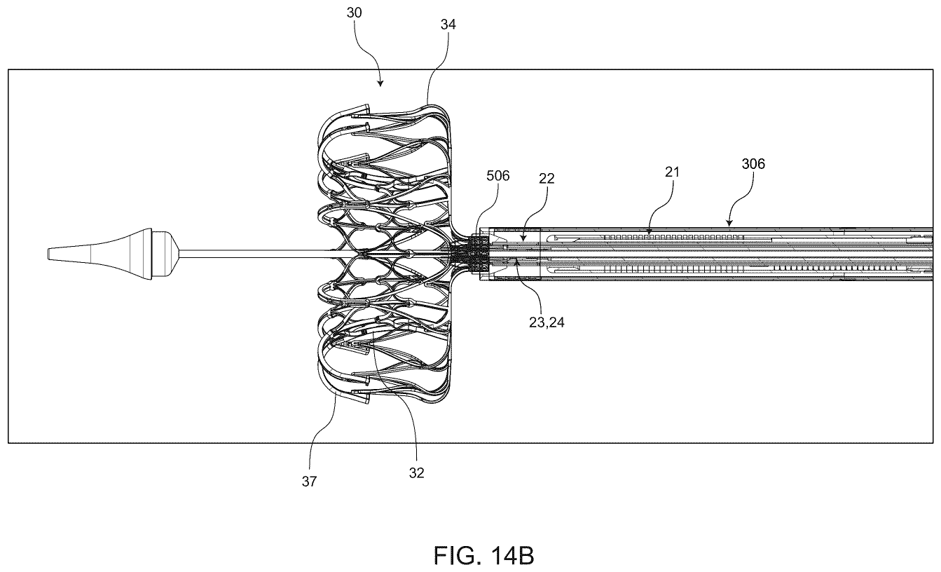

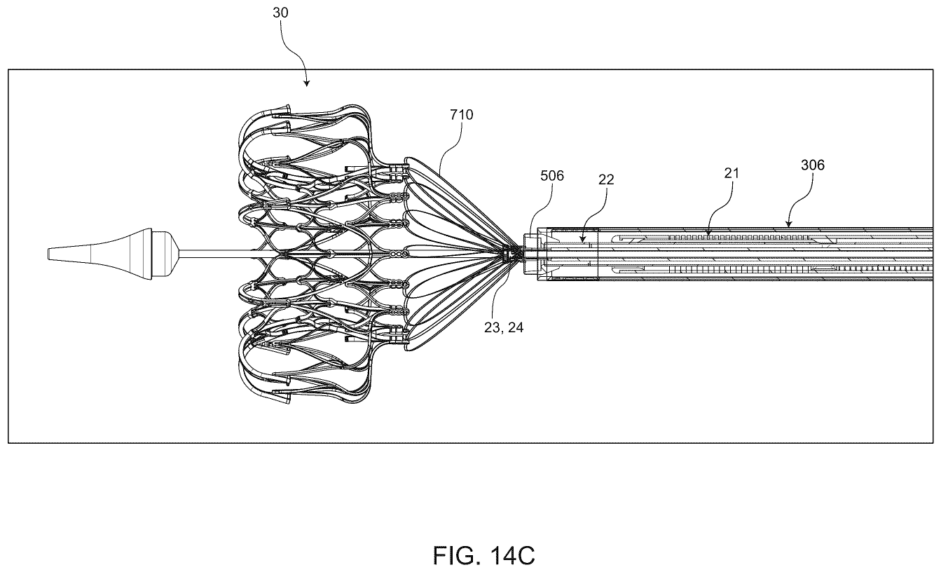

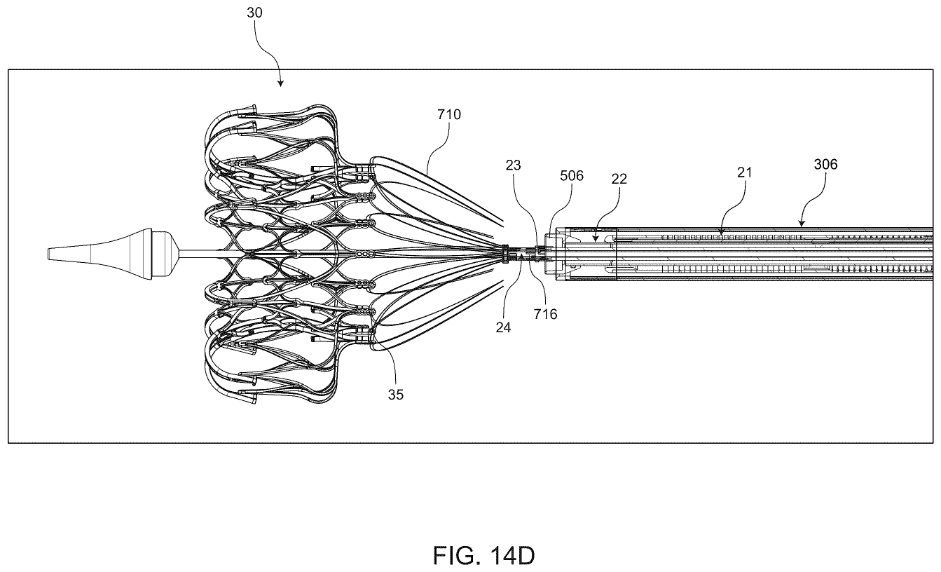

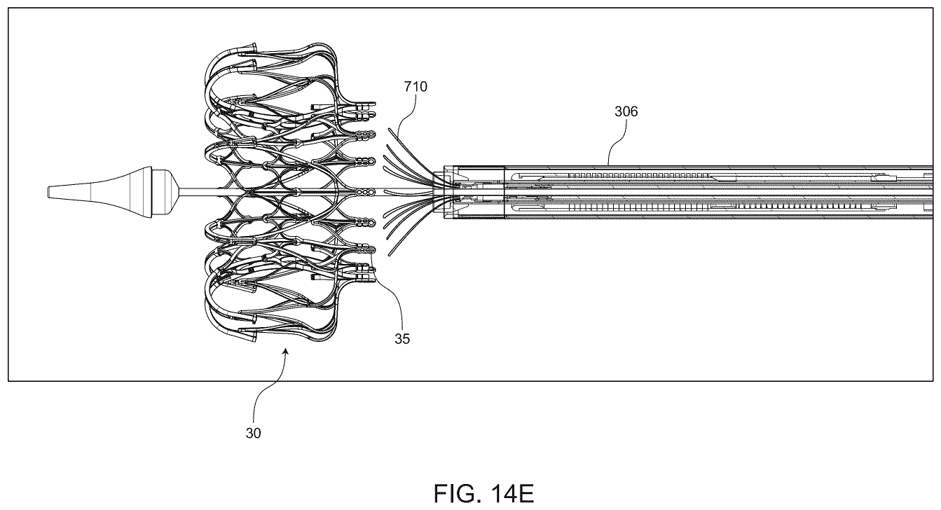

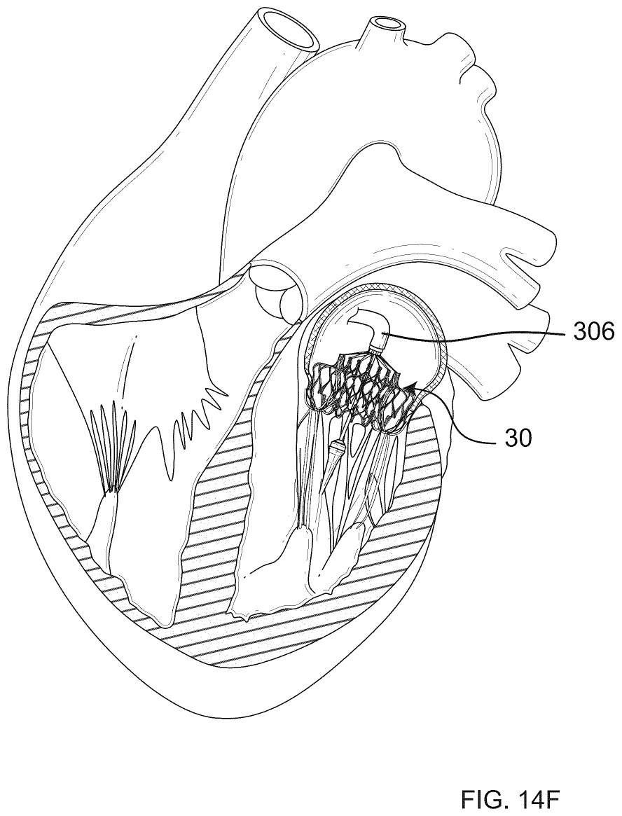

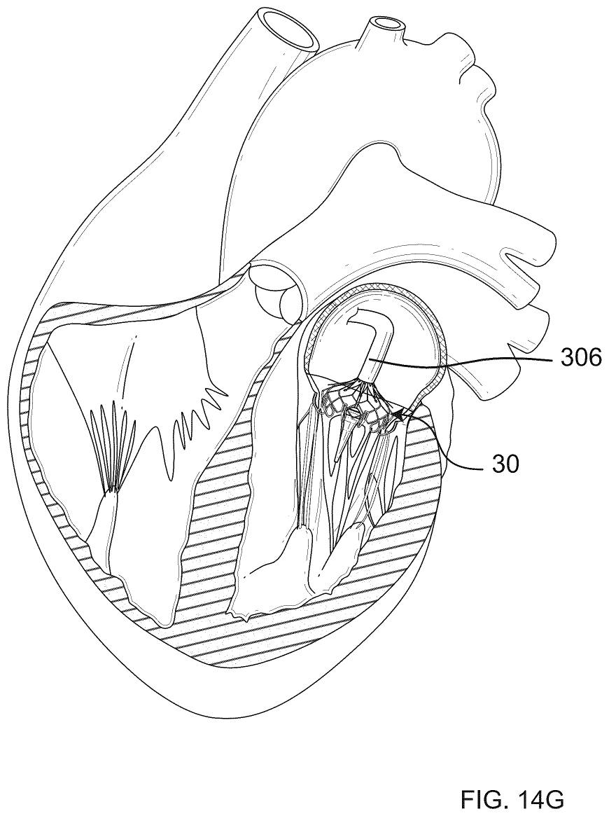

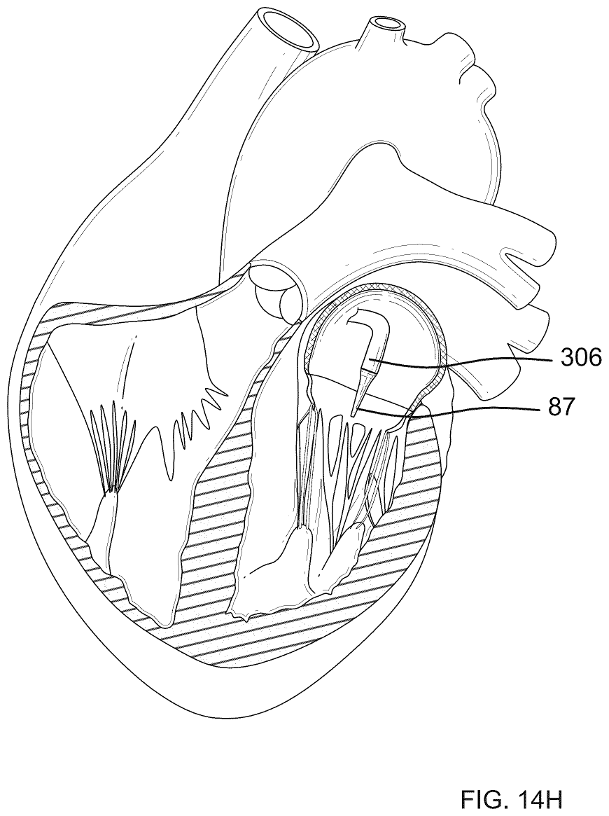

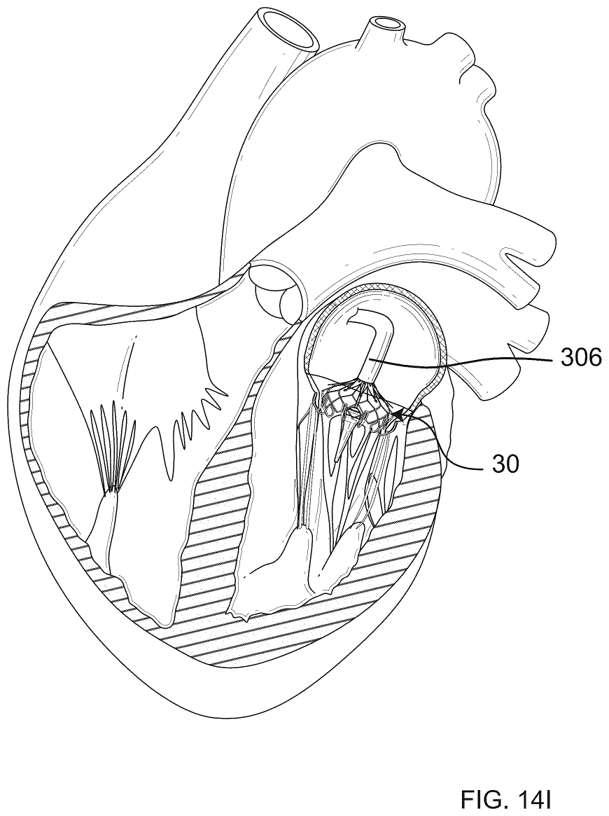

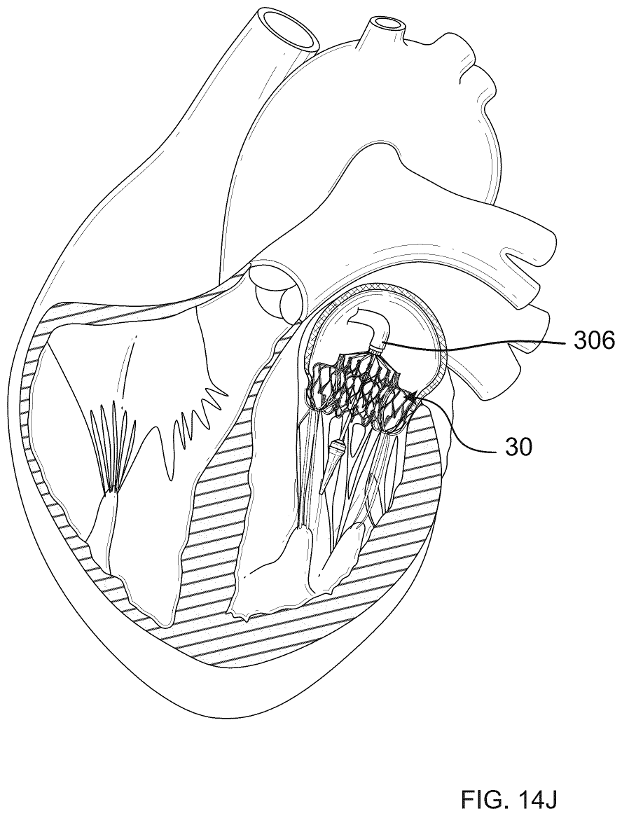

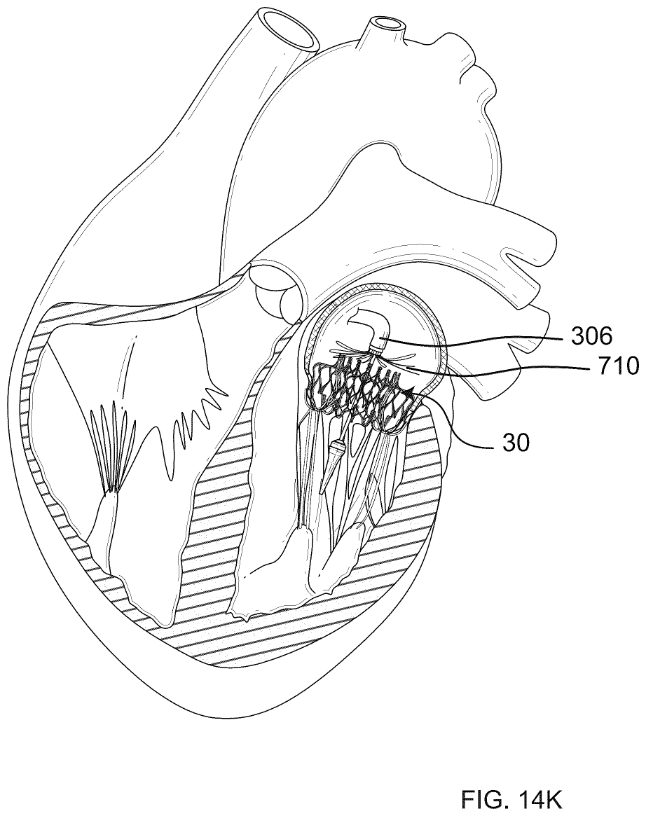

FIGS. 14A-14E illustrate various steps of deployment of the valve prosthesis using the delivery device described herein, with a focus on the positioning of the various subassemblies of the delivery device with respect to each other and with respect to the valve prosthesis at the different steps. FIGS. 14F to 14K illustrate various steps of deployment and recapture of the valve prosthesis using the delivery device described herein shown with reference to an example implantation location within the heart.

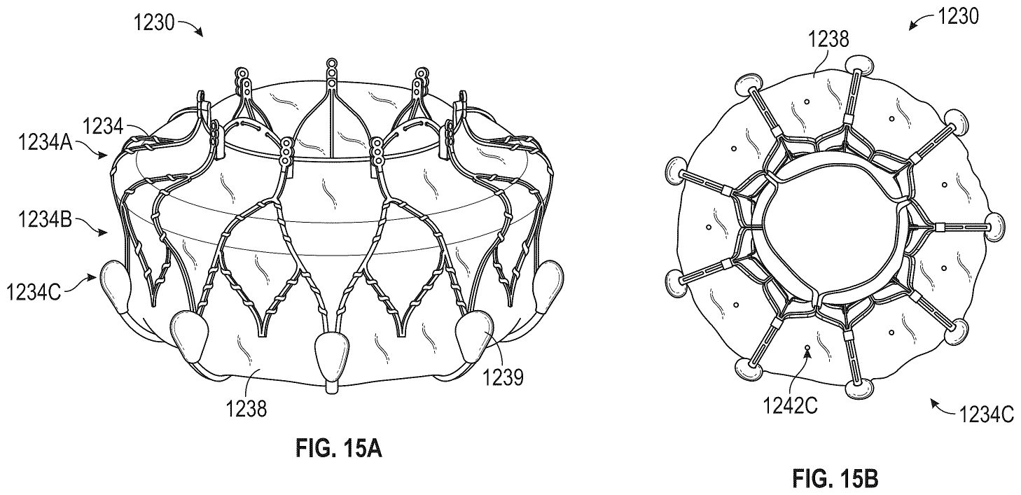

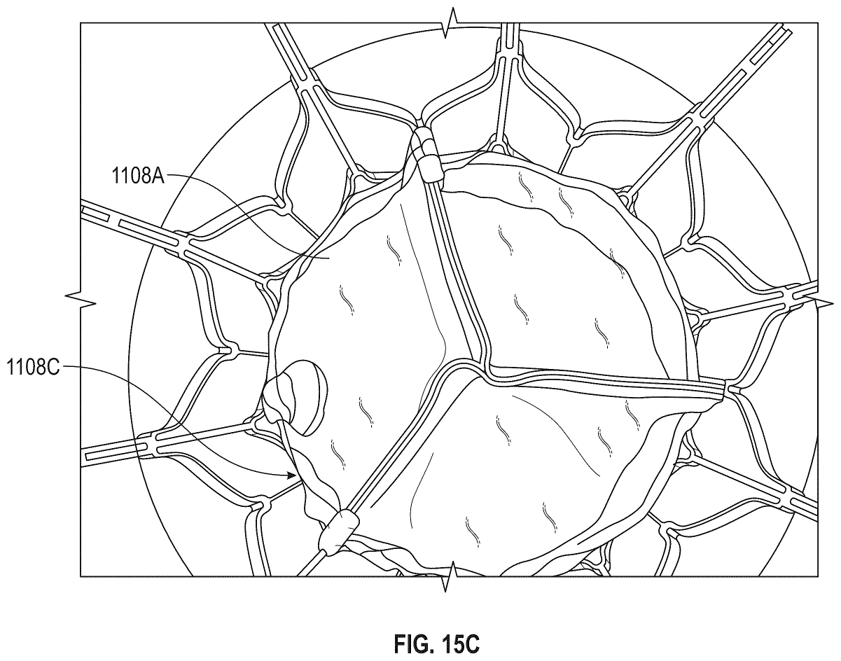

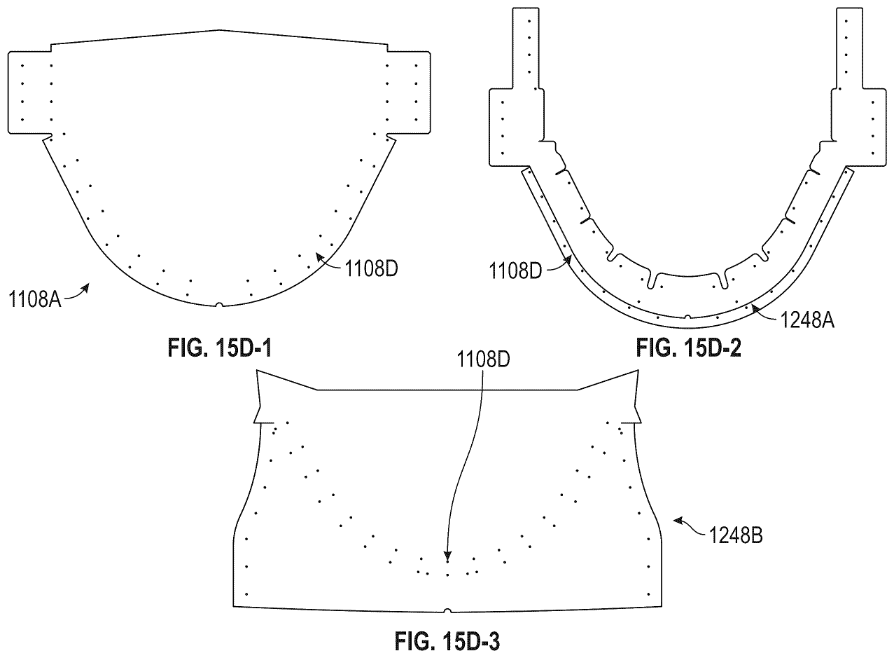

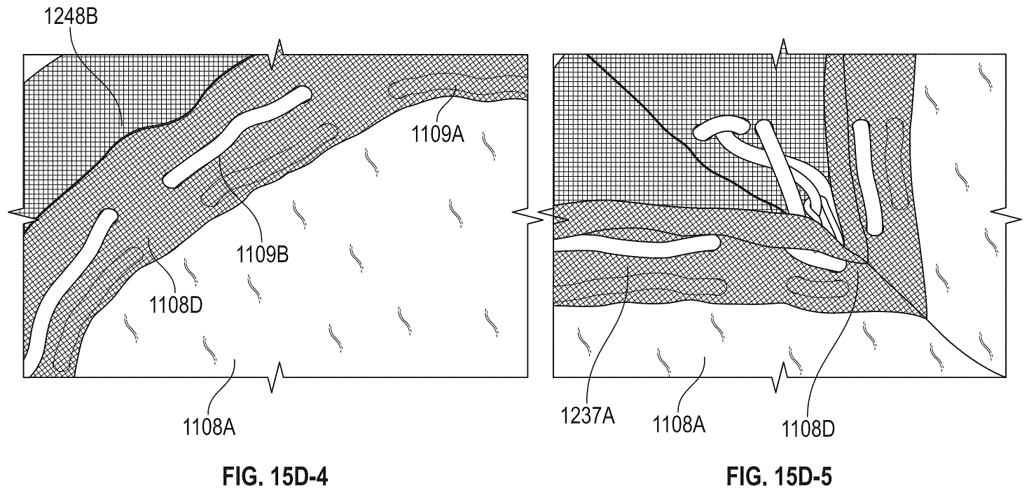

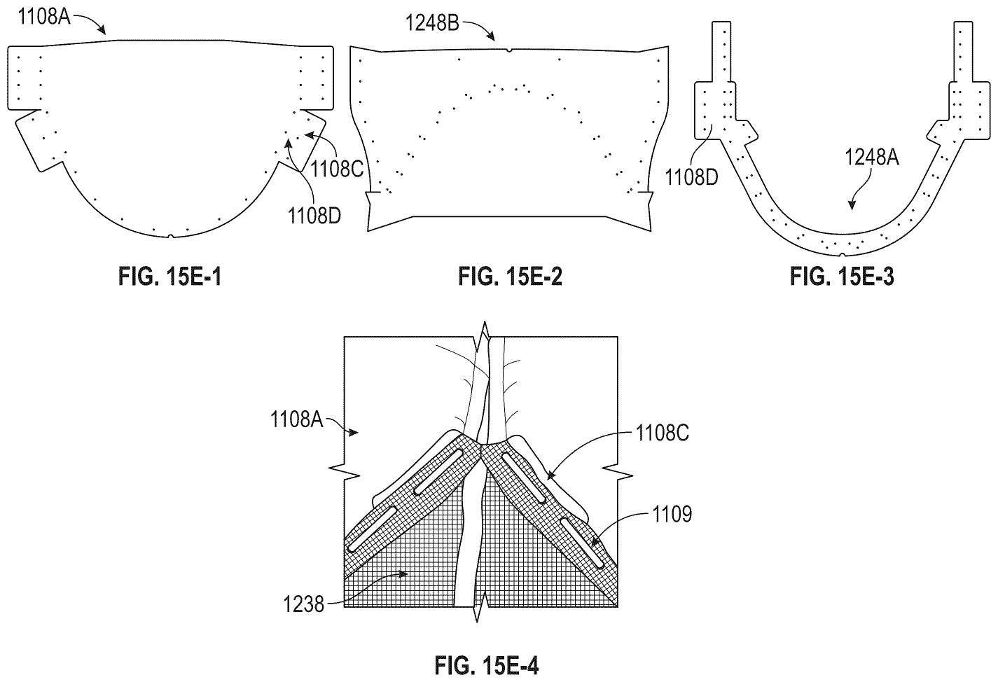

FIG. 15A shows a side perspective view of a configuration of a fully-assembled dual-frame valve prosthesis including a skirt assembly and padding.

FIG. 15B shows a side view of the fully-assembled dual-frame valve prosthesis of FIG. 15A.

FIG. 15C shows a prosthetic leaflet stitched to an inner frame of the dual-frame valve prosthesis.

FIGS. 15D-1 to 15D-5 and 15E-1 to 15E-4 show double stitching applied to a prosthetic leaflet to securely attach the prosthetic leaflet to an inner frame of the dual-frame valve prosthesis.

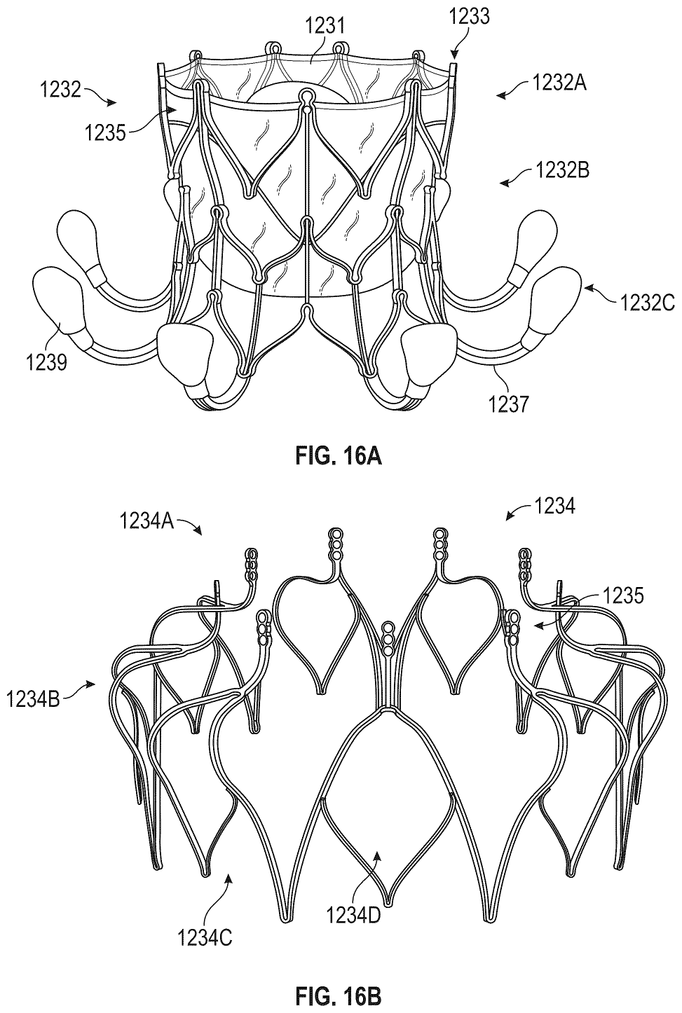

FIG. 16A shows a side perspective view of an inner frame of the dual-frame valve prosthesis of FIGS. 15A and 15B. FIG. 16B shows a side perspective view of an outer frame of the dual-frame valve prosthesis of FIGS. 15A and 15B.

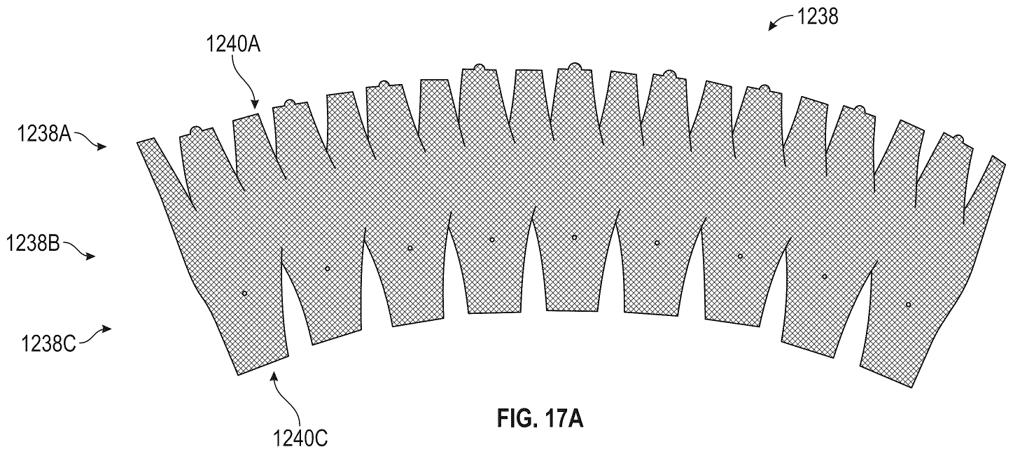

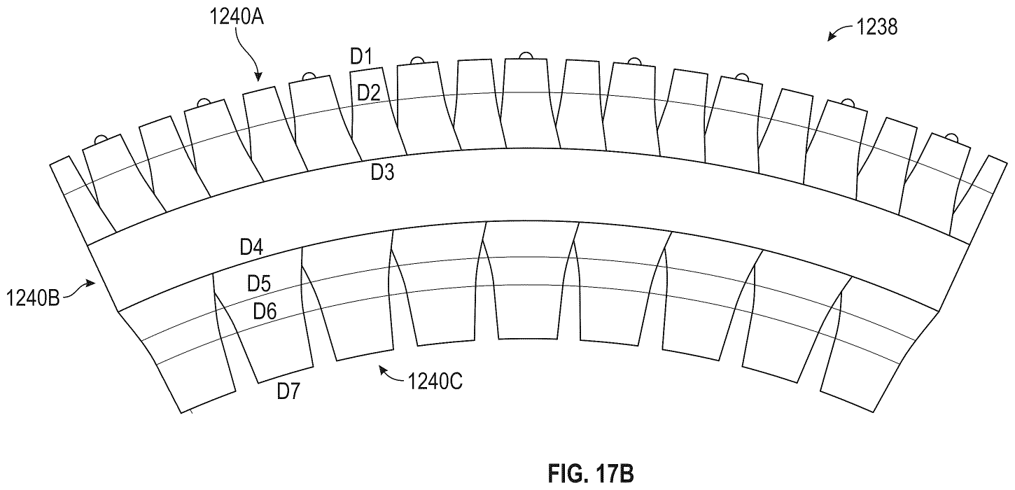

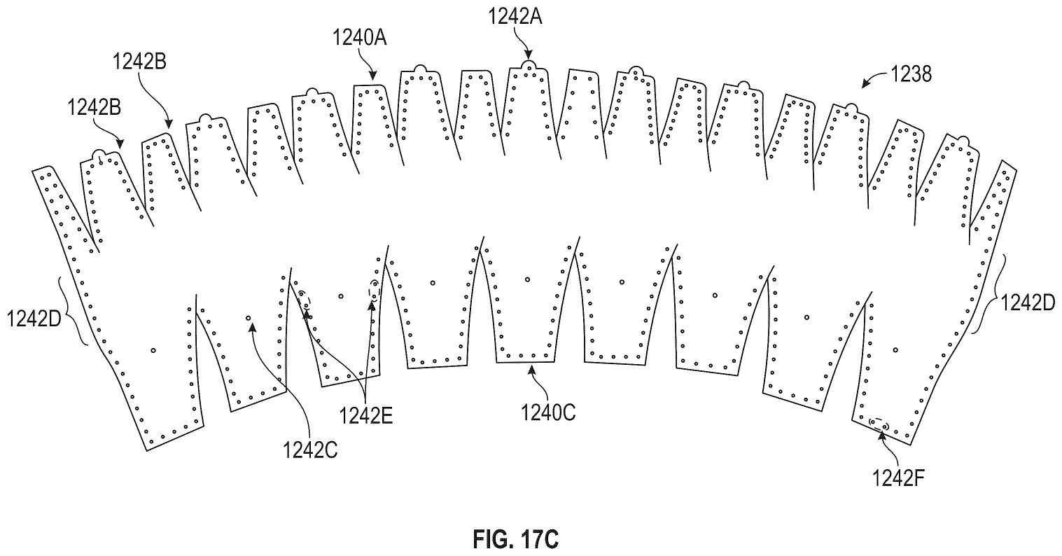

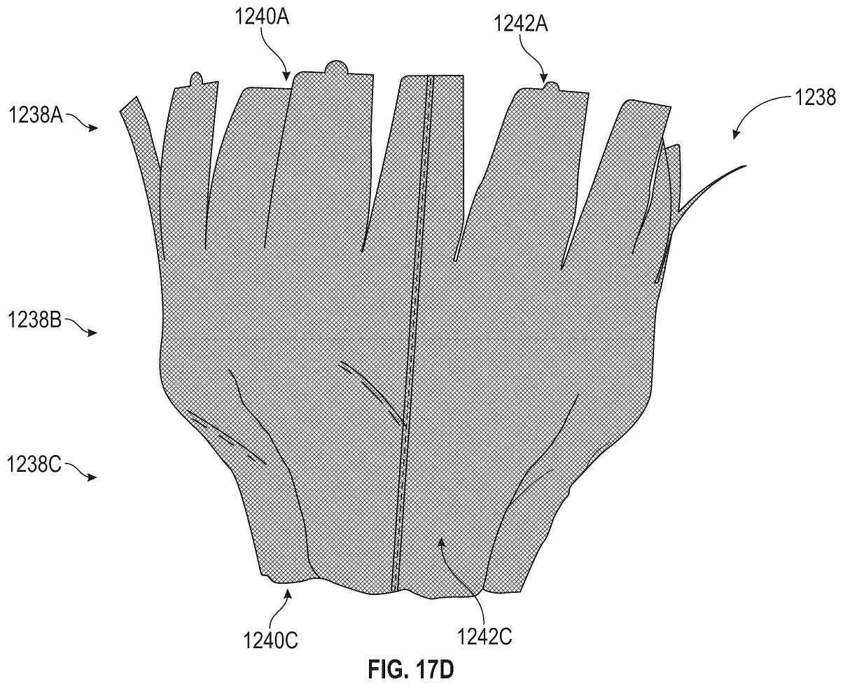

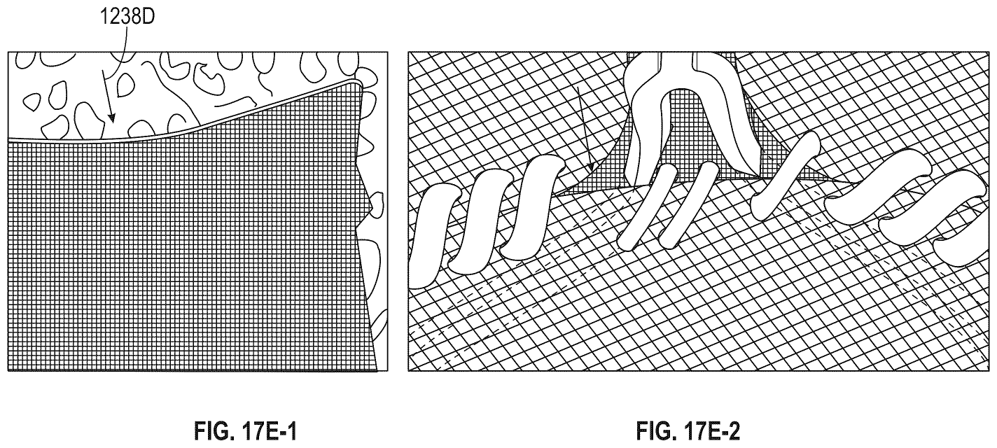

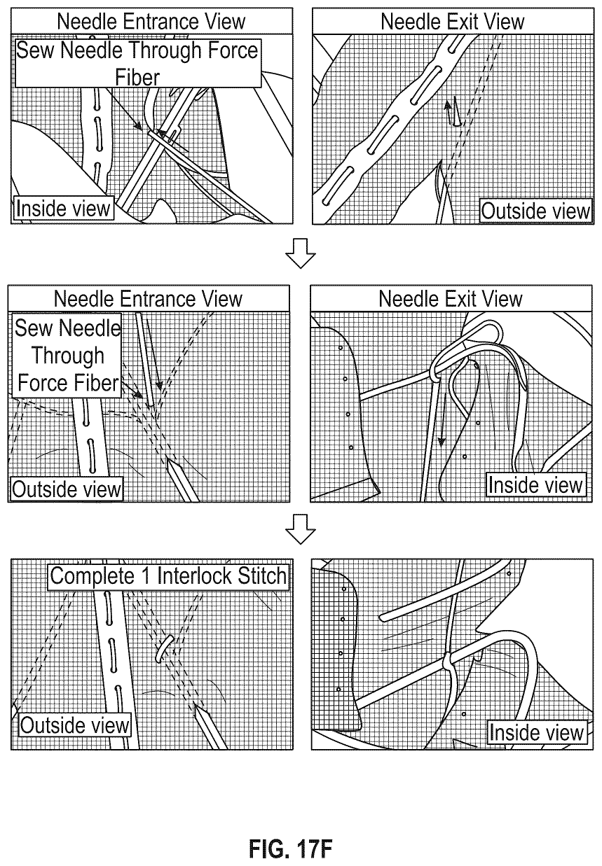

FIGS. 17A to 17C show the skirt assembly of the dual-frame valve prosthesis of FIGS. 15A and 15B in a flat configuration. FIG. 17D shows a side view of the skirt assembly of the dual-frame valve prosthesis of FIGS. 15A and 15B in a partially folded configuration.

FIGS. 17E-1 and 17E-2 show softened edges of cloth material used for the skirt assembly of FIGS. 17A to 17D.

FIG. 17F shows a process of applying an interlocking stitch of the cloth material used for the skirt assembly of FIGS. 17A to 17D to eliminate knots.

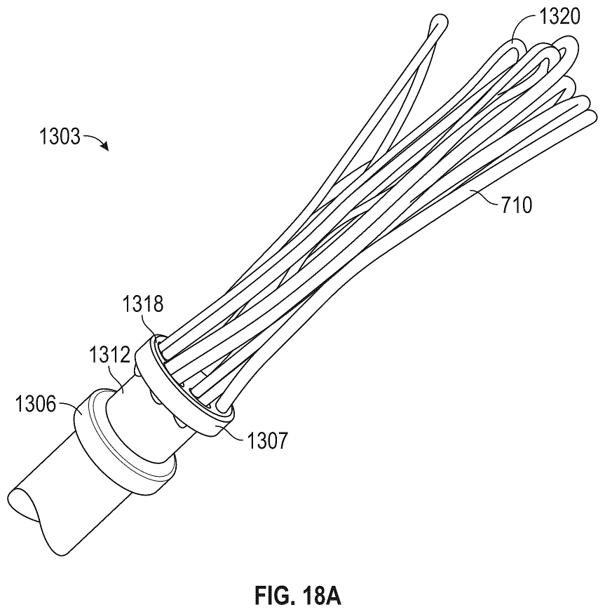

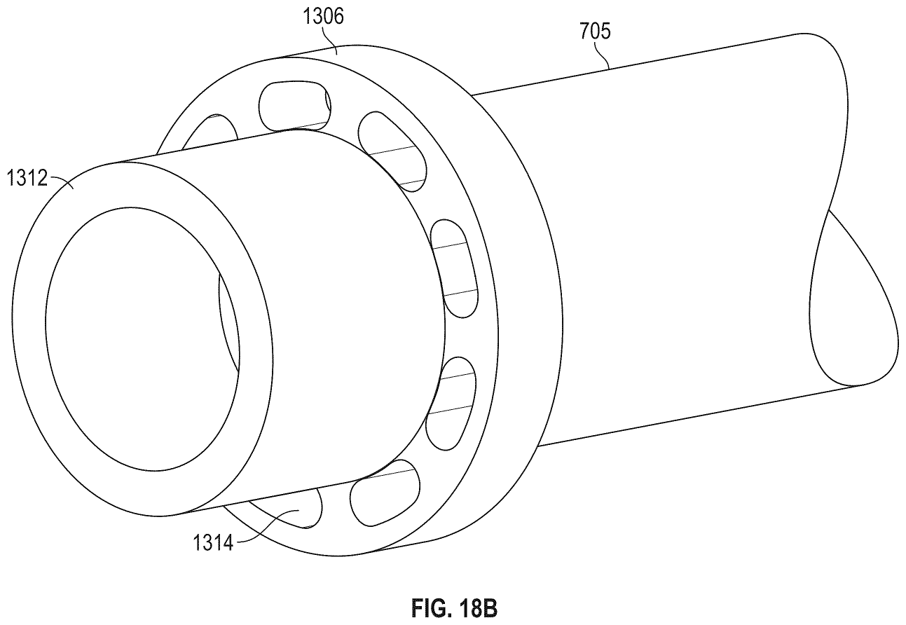

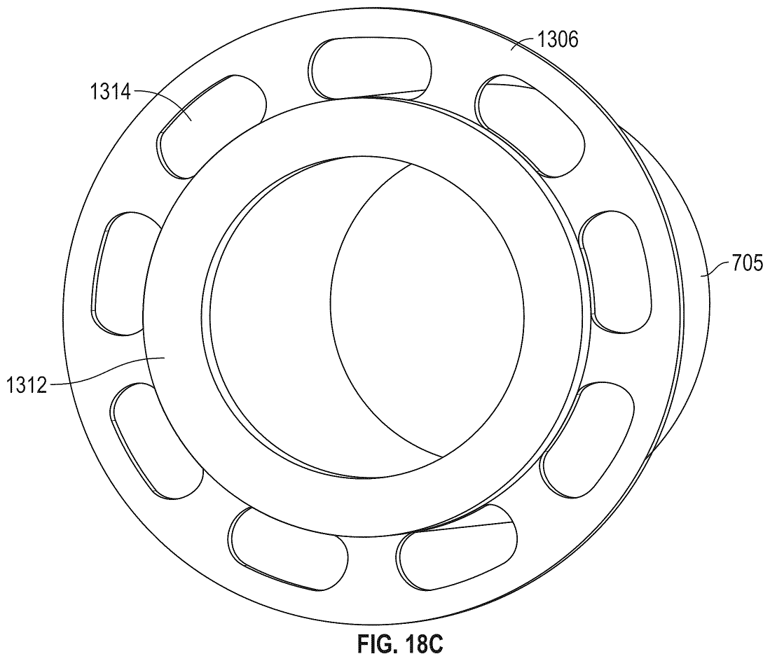

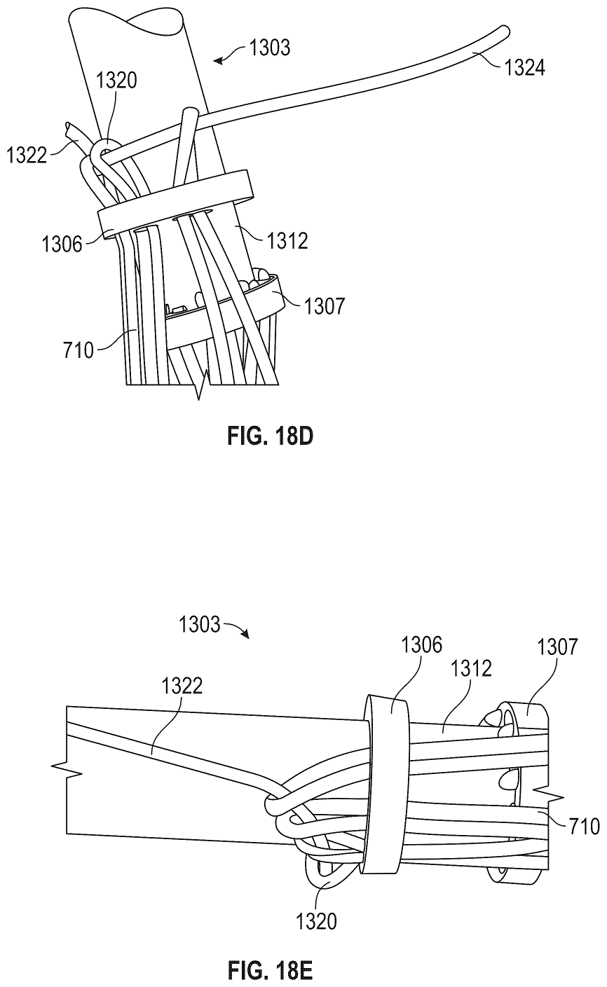

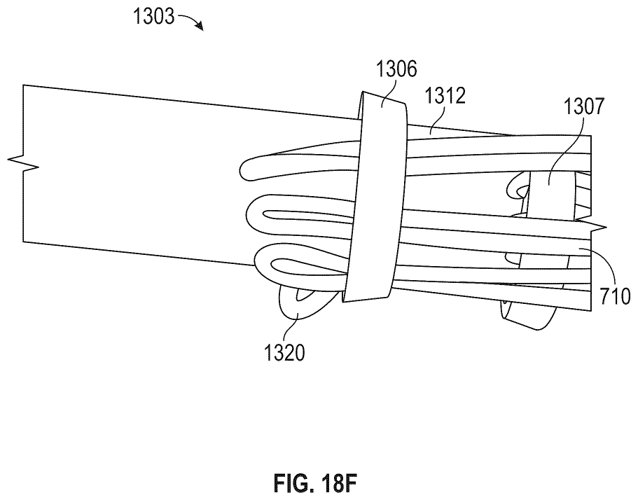

FIG. 18A shows a close-up view of a distal end portion of a configuration of a manifold subassembly with suture or tether loops assembled thereto.

FIG. 18B shows a perspective side view of the distal end portion of the configuration of the manifold subassembly of FIG. 18A. FIG. 18C shows a perspective bottom view of the distal end portion of the configuration of the manifold subassembly of FIG. 18A. FIG. 18D shows a perspective view of a tether or a suture arrangement being secured to the distal end portion of the configuration of the manifold subassembly of FIG. 18A. FIGS. 18E and 18F show a perspective view of the manifold subassembly illustrating how a retention portion of the tether or suture arrangement can be removed from the distal end portion of the configuration of the manifold subassembly of FIG. 18A.

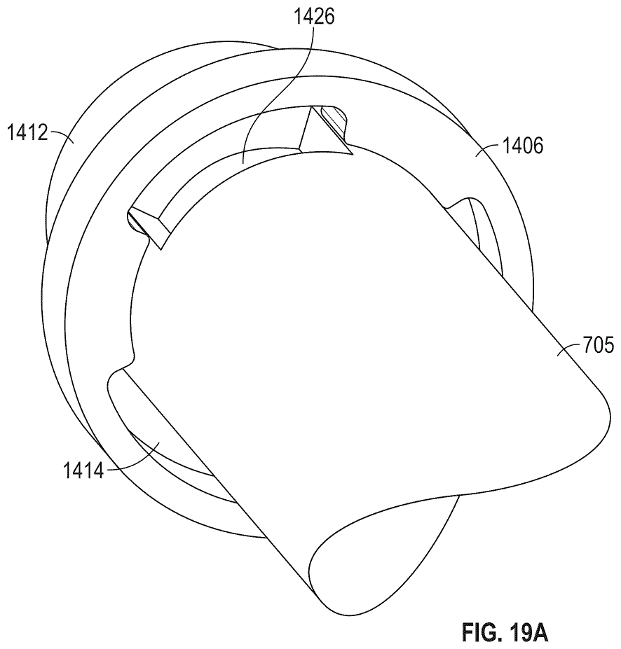

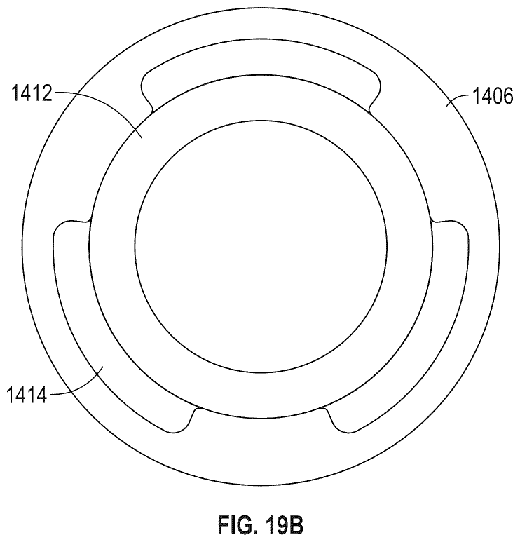

FIG. 19A shows a perspective side view of a distal end portion of another configuration of a manifold subassembly. FIG. 19B shows a plan view of the distal end portion of the configuration of the manifold subassembly of FIG. 19A.

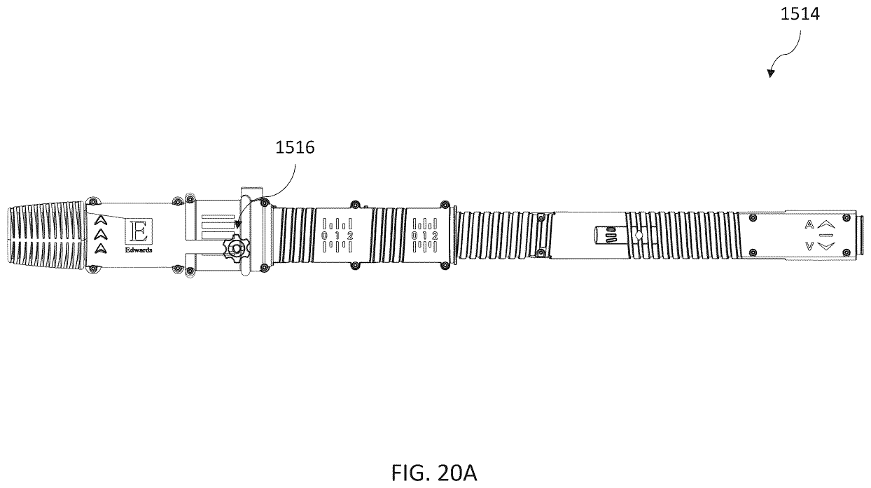

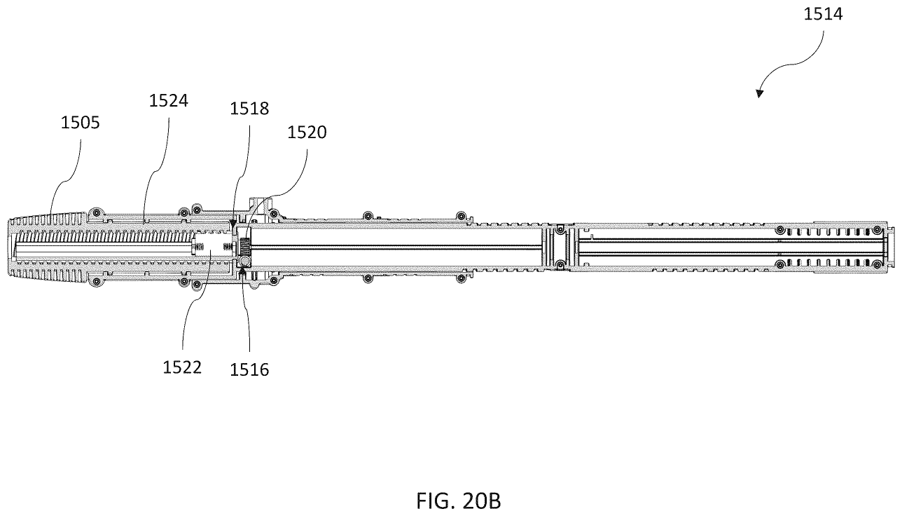

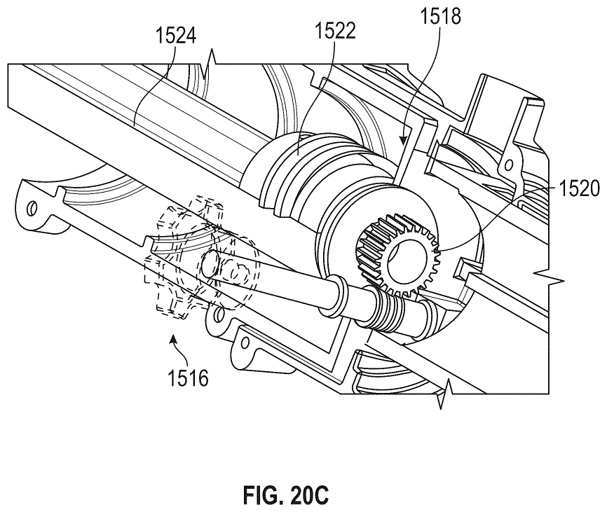

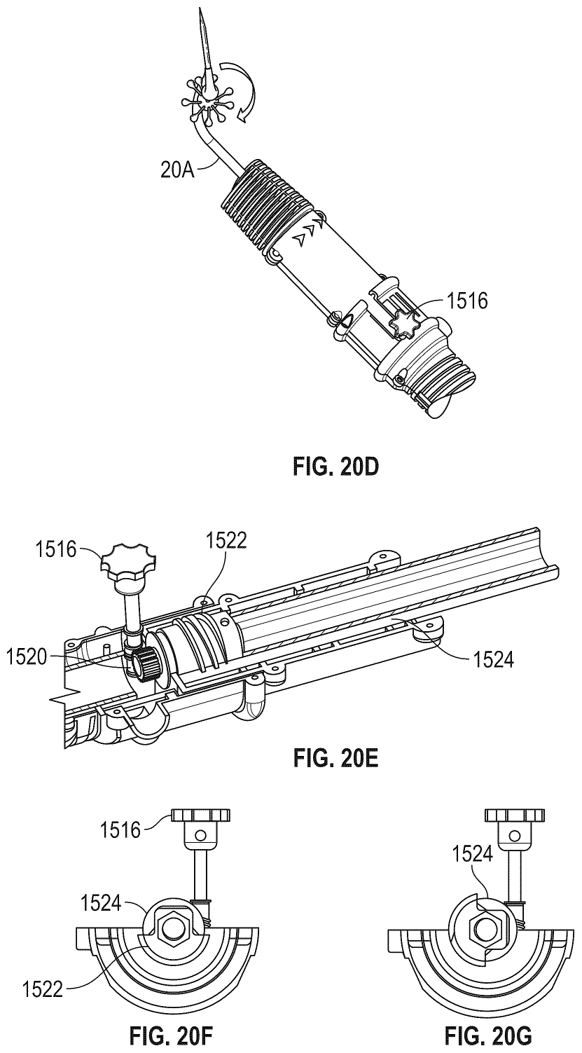

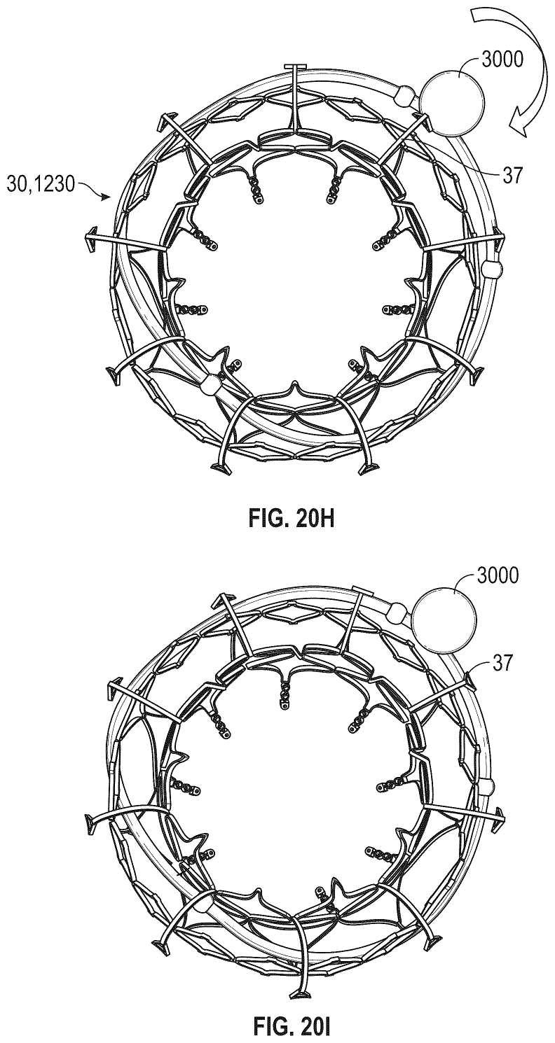

FIG. 20A shows a side view of a configuration of a handle of a delivery device. FIG. 20B shows a side cross-section view of the handle of FIG. 20A. FIG. 20C shows a close-up cross-section view of the handle of FIG. 20A.

FIGS. 20D, 20E, 20F and 20G illustrate an orientation mechanism of FIG. 20C connected to an outer lumen within which a dual-frame valve prosthesis rotates to facilitate clocking of the prosthesis at a desired implantation location. FIGS. 20H and 201 schematically illustrate a clocking mechanism utilizing direct fluoroscopic visualization.

FIG. 21 shows a perspective view of a configuration of a handle of a delivery device.

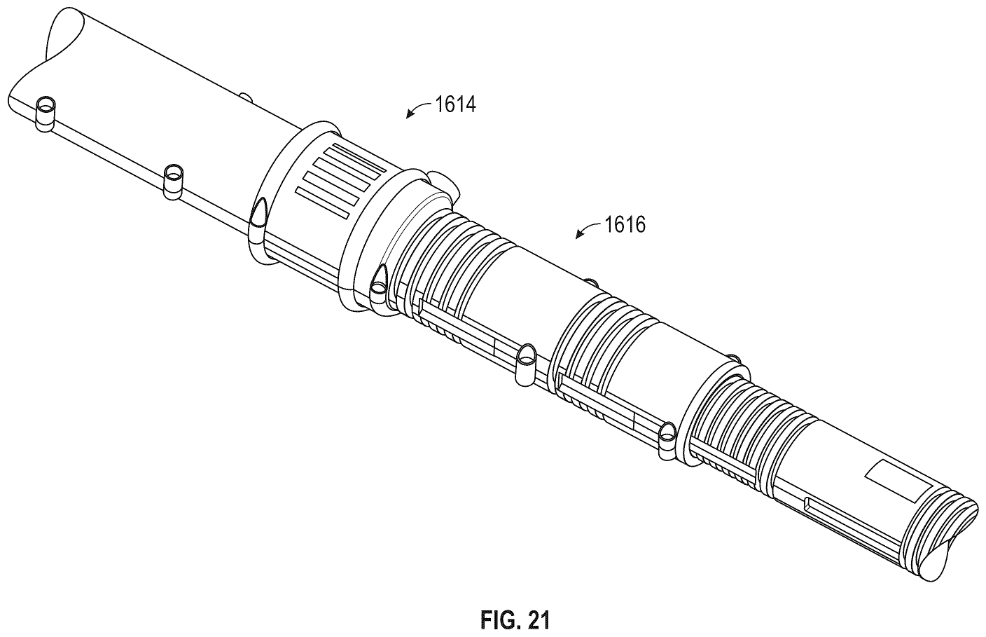

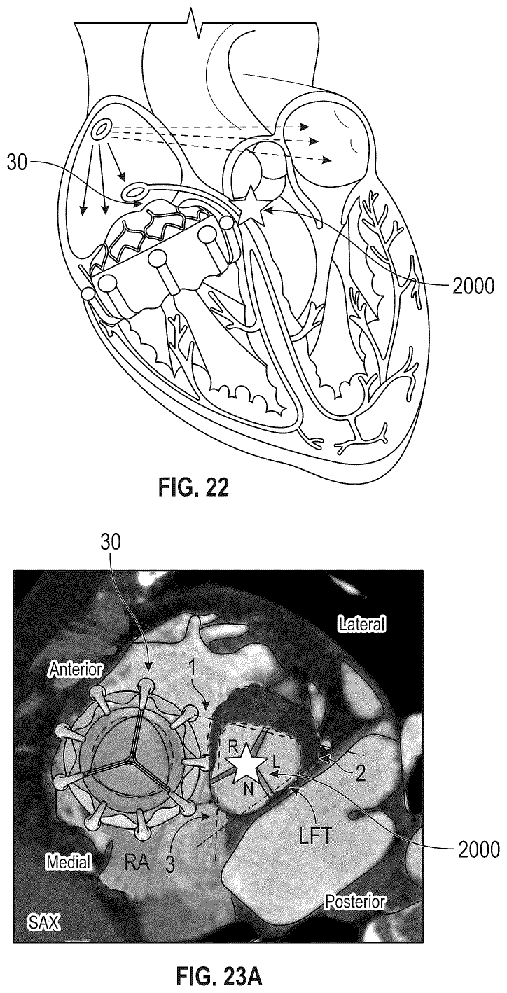

FIG. 22 shows a configuration of an implant within a heart of a patient.

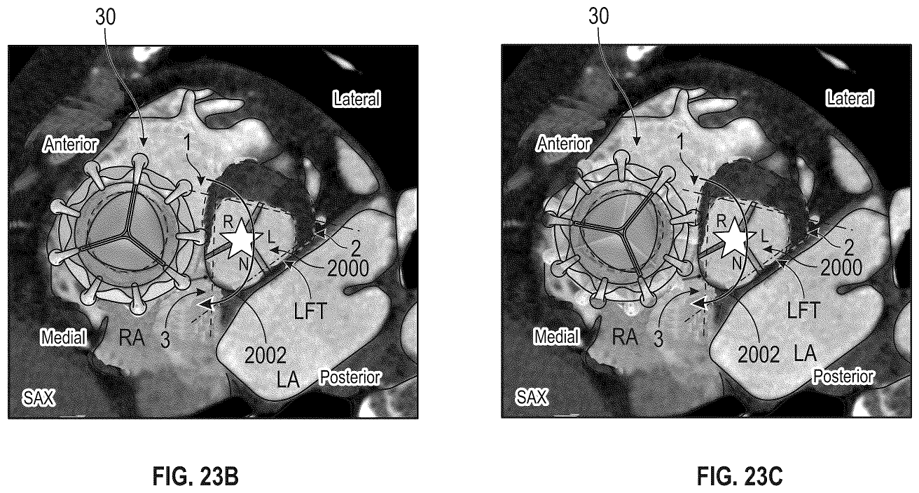

FIGS. 23A to 23C show the implant shown in FIG. 22 being rotated within the heart of the patient.

### DETAILED DESCRIPTION

The present specification and drawings provide aspects and features of the disclosure in the context of several embodiments of replacement heart valves, delivery systems and methods that are configured for use in the vasculature of a patient, such as for replacement of natural heart valves in a patient. These embodiments may be discussed in connection with replacing specific valves such as the patient's aortic, tricuspid, or mitral valve. However, it is to be understood that the features and concepts discussed herein can be applied to products other than heart valve implants. For example, the controlled positioning, deployment, and securing features described herein can be applied to medical implants, for example other types of expandable prostheses, for use elsewhere in the body, such as within an artery, a vein, or other body cavities or locations. In addition, particular features of a valve, delivery system, etc. should not be taken as limiting, and features of any one embodiment discussed herein can be combined with features of other embodiments as desired and when appropriate. While certain of the embodiments described herein are described in connection with a transfemoral delivery approach, it should be understood that these embodiments can be used for other delivery approaches such as, for example, transapical or transjugular approaches. Moreover, it should be understood that certain of the features described in connection with some embodiments can be incorporated with other embodiments, including those which are described in connection with different delivery approaches.

### Delivery System

FIG. 1 illustrates an embodiment of a delivery system 10. The delivery system 10 can be used to deploy a prosthesis, such as a replacement heart valve, to a location within a body of a subject (e.g., human or veterinary subject). Replacement heart valves can be delivered to a subject's heart mitral or tricuspid valve annulus or other heart valve location in various manners, such as by open surgery, minimally-invasive surgery, and percutaneous or transcatheter delivery through the subject's vasculature. Example transfemoral approaches are described further in U.S. Pat. Publ. No. 2015/0238315, published Aug. 27, 2015, the entirety of which is hereby incorporated by reference in its entirety. While the delivery system 10 is described in connection with a percutaneous delivery approach, and more specifically a transfemoral delivery approach, it should be understood that features of delivery system 10 can be applied to other delivery approaches, including delivery systems for a transapical delivery approach.

The delivery system 10 can be used to deploy a prosthesis, such as a replacement heart valve as described elsewhere in this specification, to a location within the body of a subject. The delivery system 10 can include multiple components, devices, or subassemblies. As shown in FIG. 1, the delivery system 10 can include a delivery device 15, a stabilizer assembly 1100, and an introducer assembly 1000 (not shown in FIG. 1 but shown in FIG. 10). The delivery device 15 includes a shaft assembly 12 and a handle 14. An implant (e.g., valve prosthesis or replacement heart valve) 30 can advantageously be pre-attached to the delivery device 15 during manufacture or assembly such that the clinician does not have to attach the implant 30 prior to use. The delivery device 15 may be configured to facilitate delivery and implantation of the implant (e.g., valve prosthesis) 30 to and at a desired target location (e.g., a mitral or tricuspid heart valve annulus). The implant (e.g., replacement heart valve) 30 may be pre-attached to or within a distal end portion of the shaft assembly 12 and removably tethered to one or more retention components of the shaft assembly 12 during manufacturing or assembly. The delivery device 15 with the pre-attached implant 30 may then be packaged, sterilized, and shipped for use by one or more clinicians. In accordance with several embodiments, the implant 30 is not supplied pre-crimped in the shaft assembly 12 delivery device 15. In other embodiments, the implant 30 is pre-loaded or supplied pre-crimped in the shaft assembly 12.

Implants for Use with Delivery System

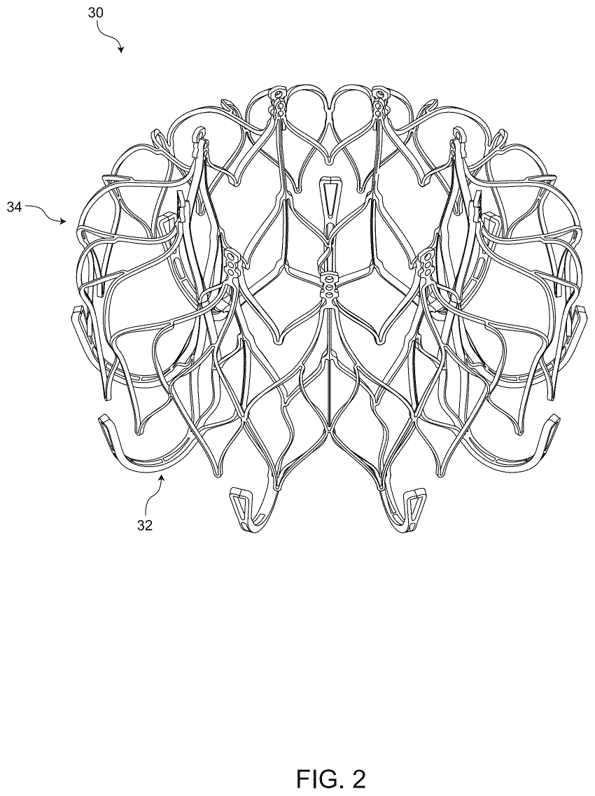

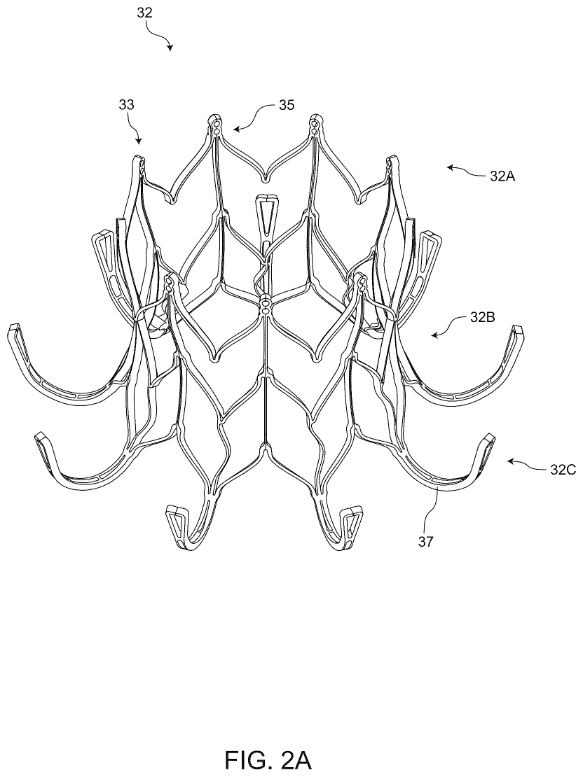

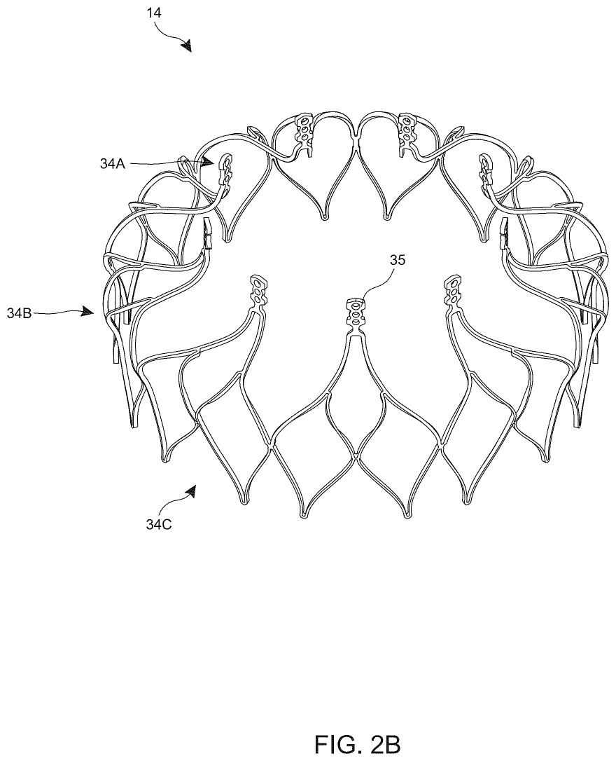

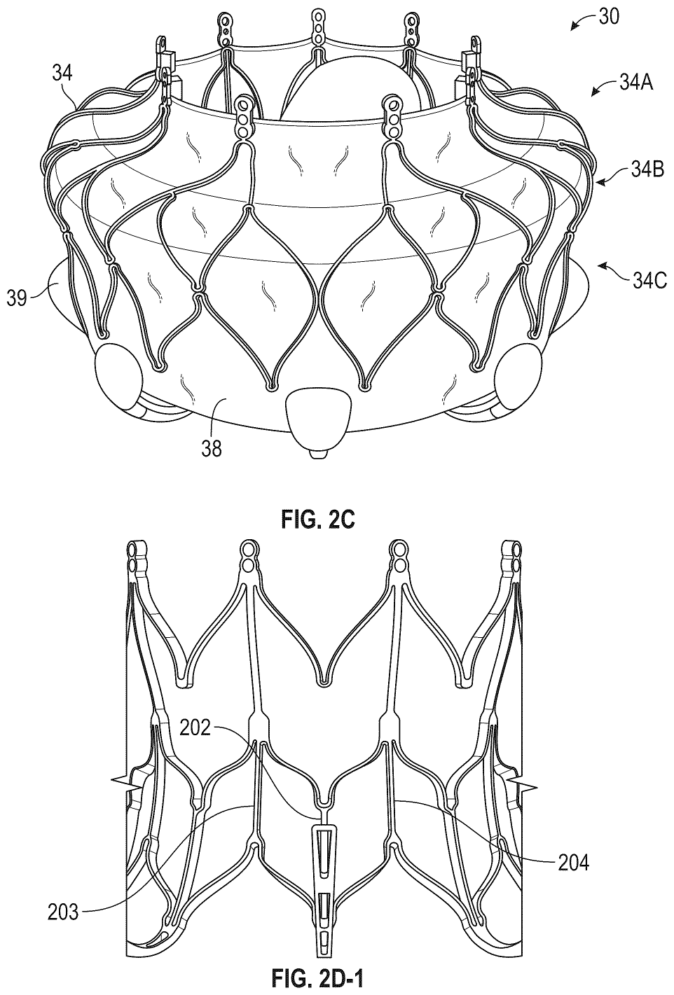

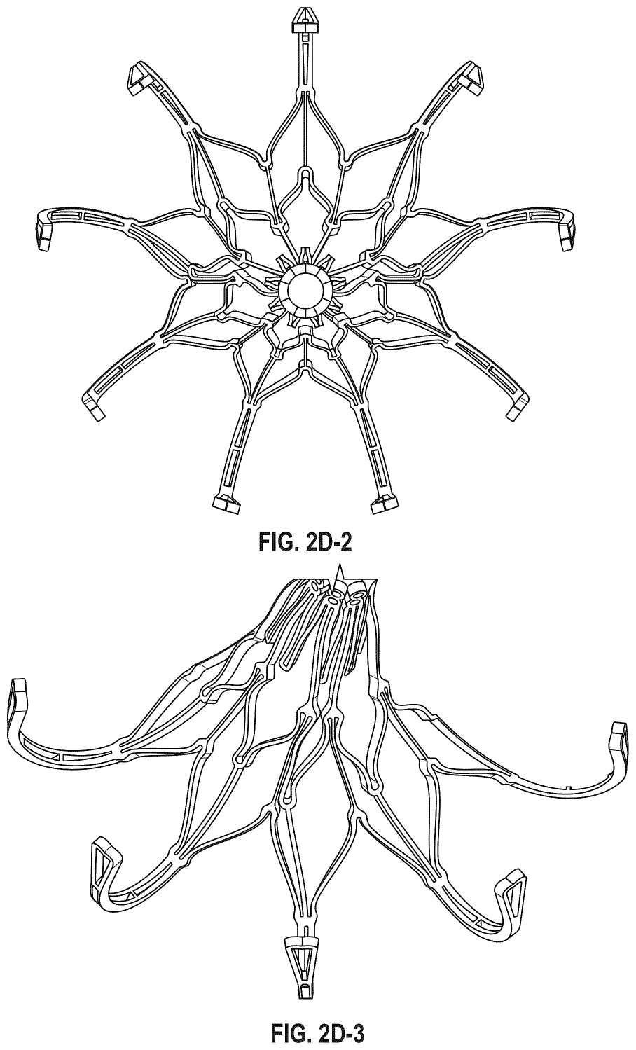

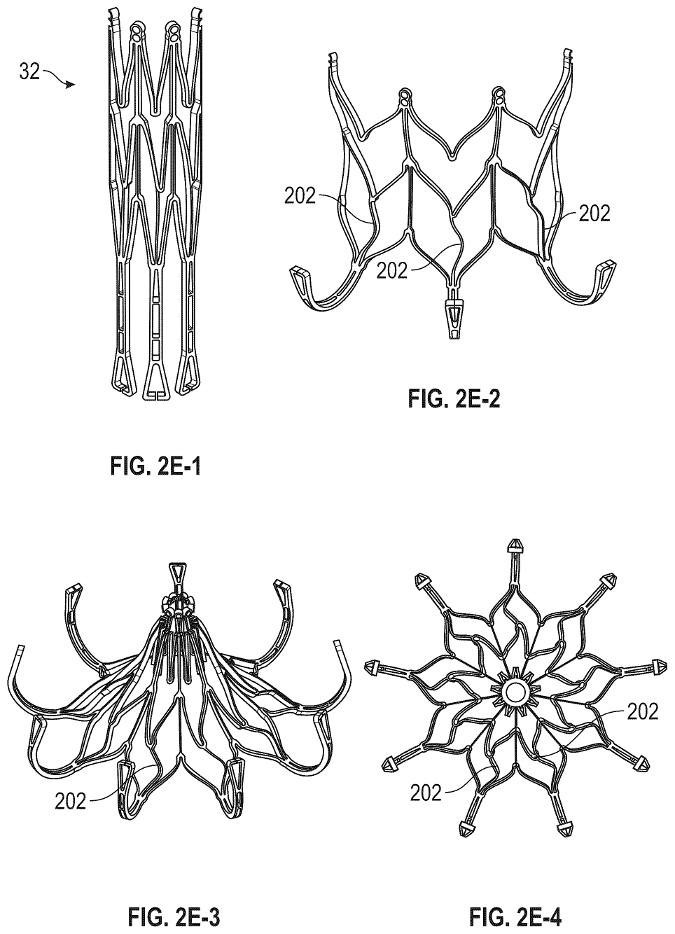

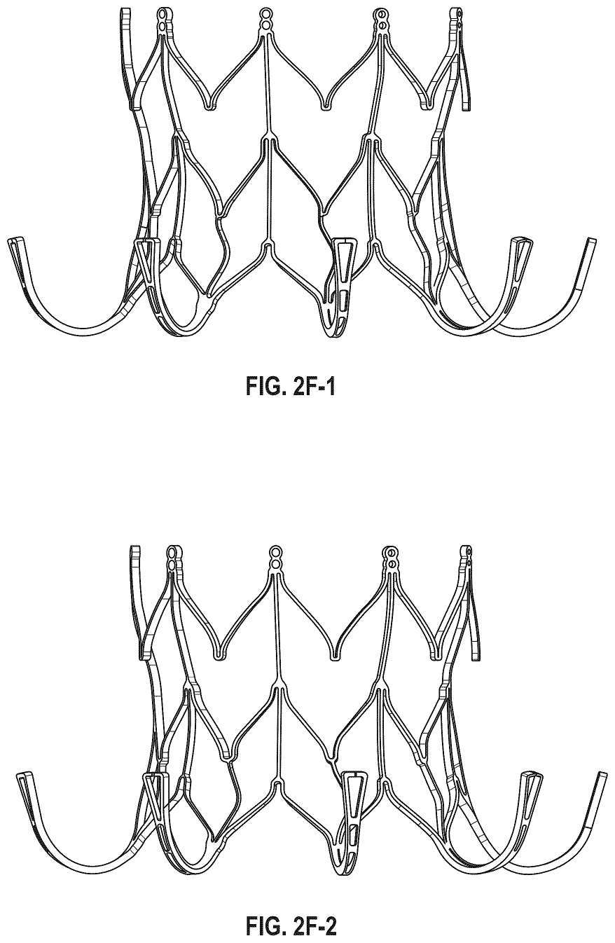

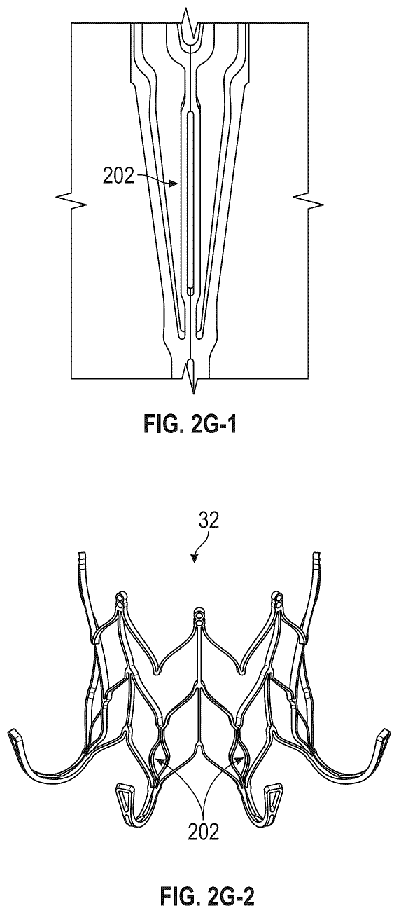

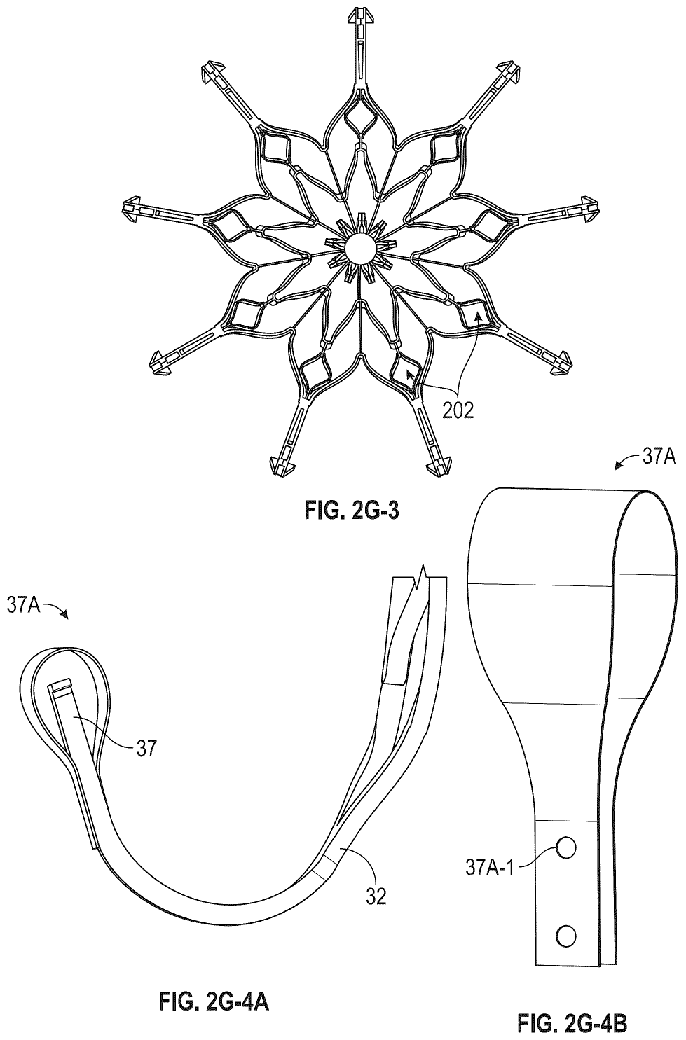

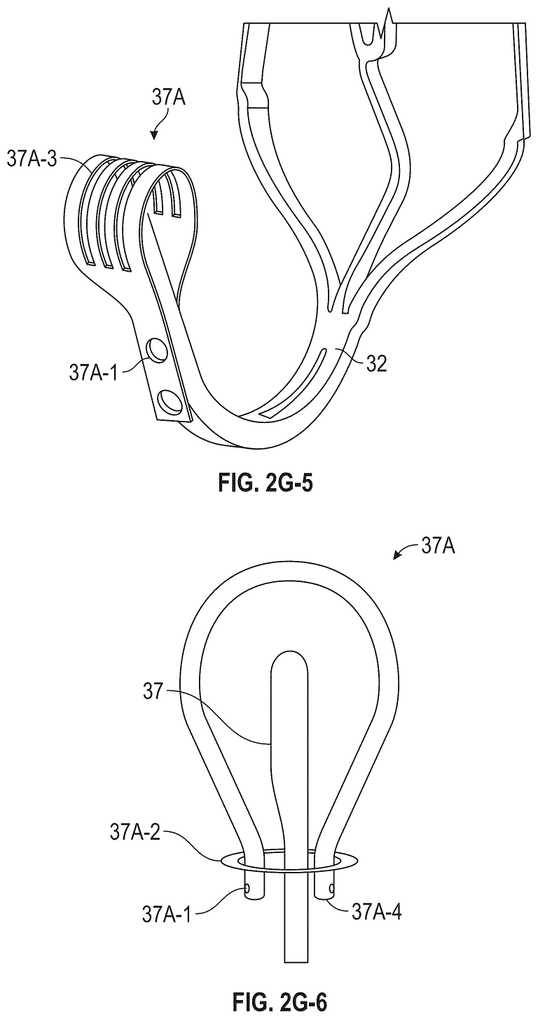

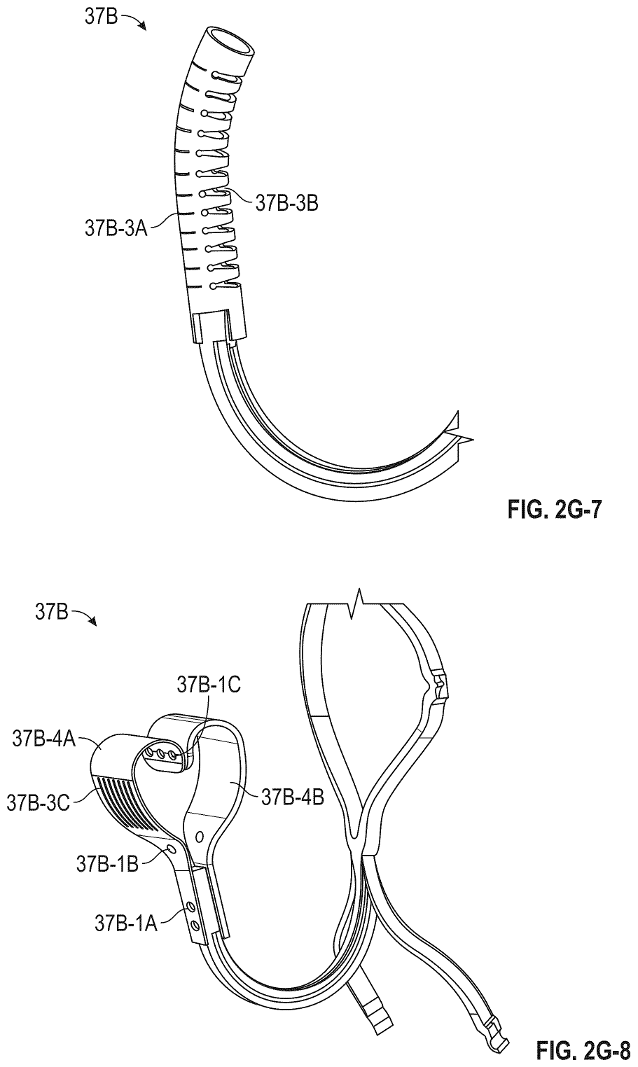

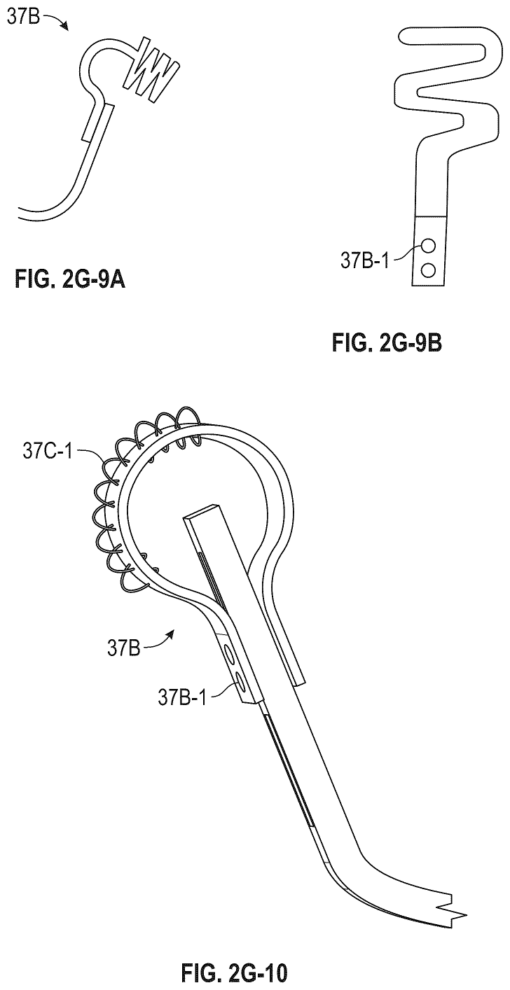

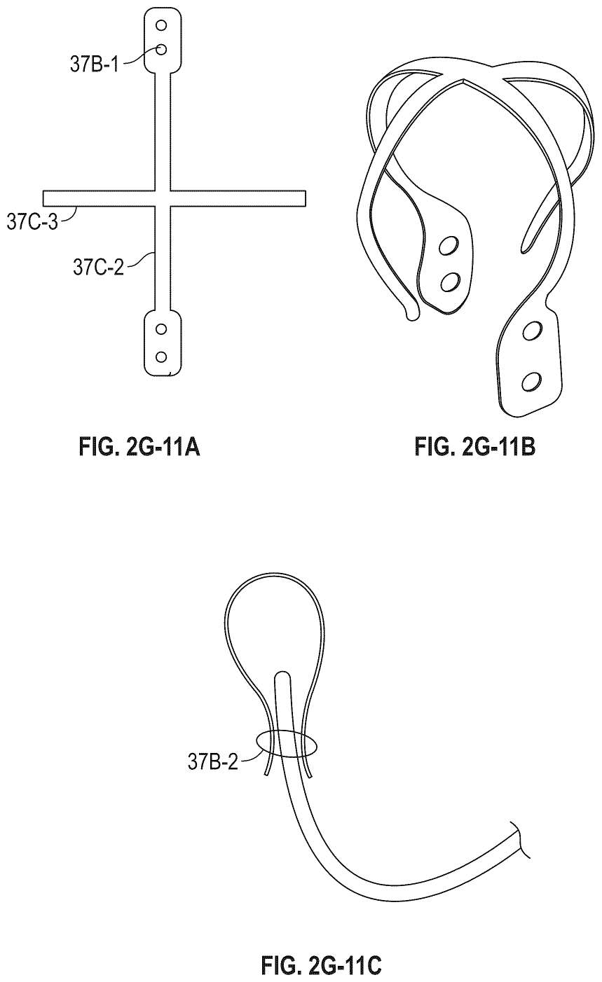

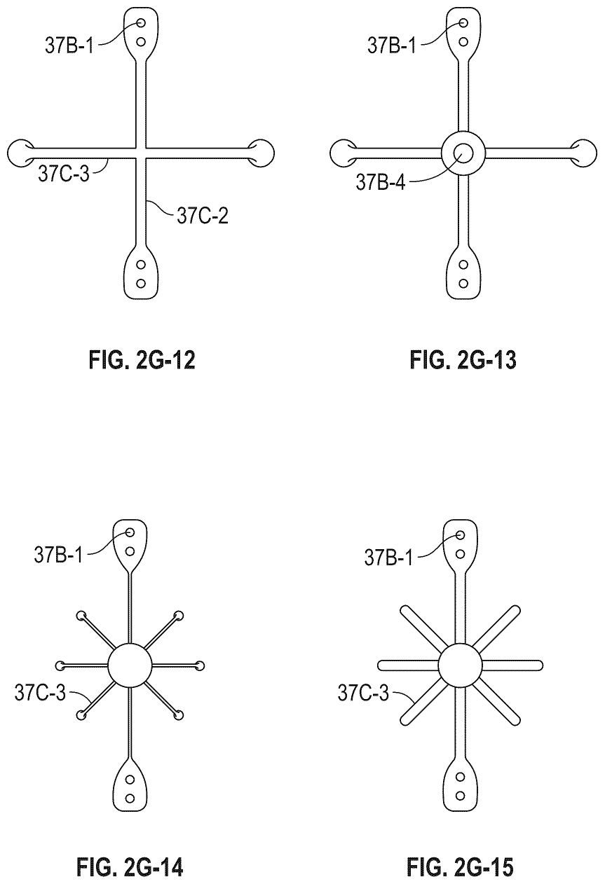

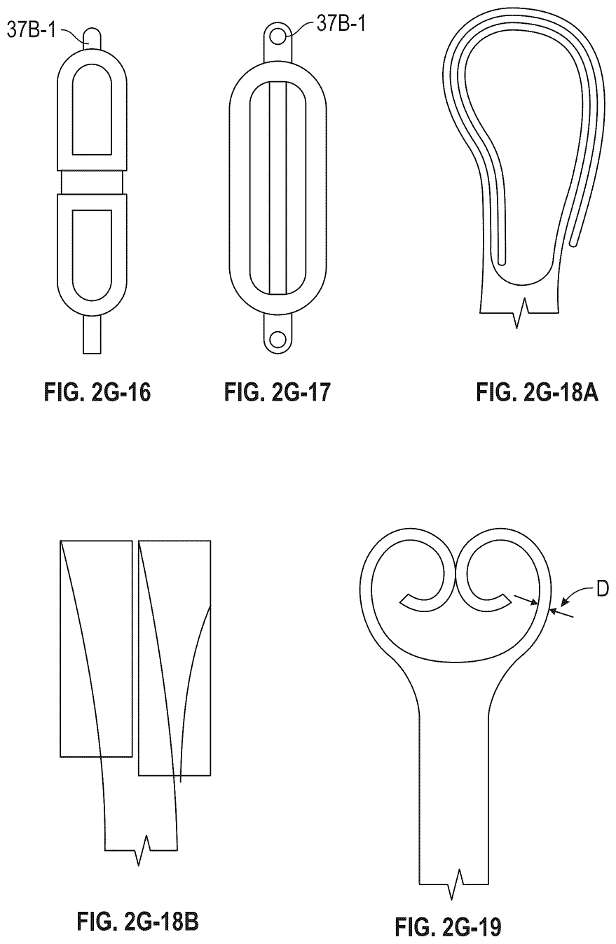

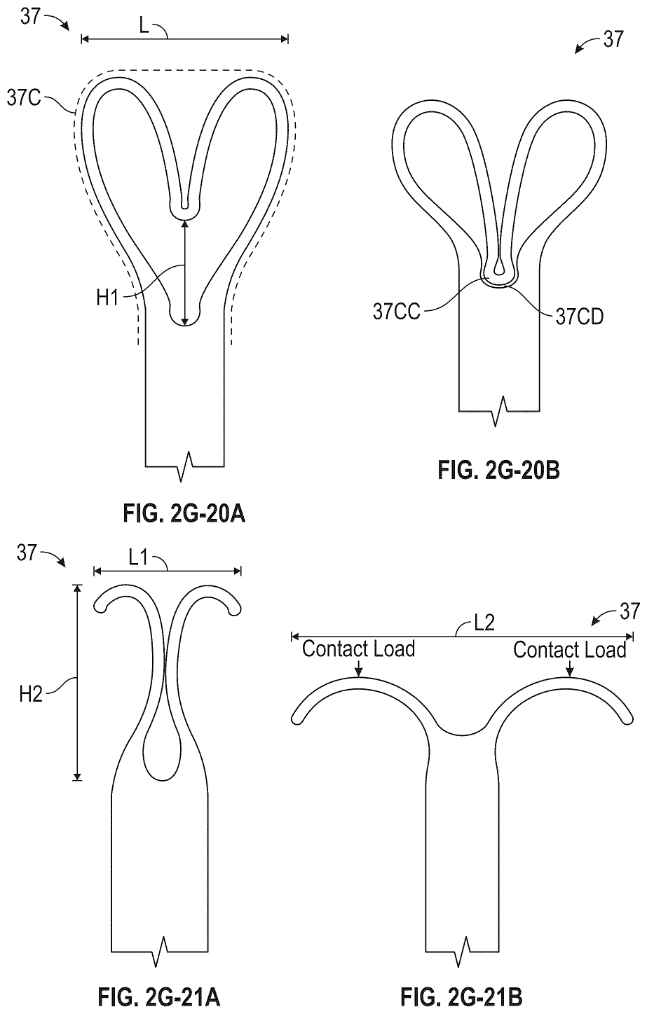

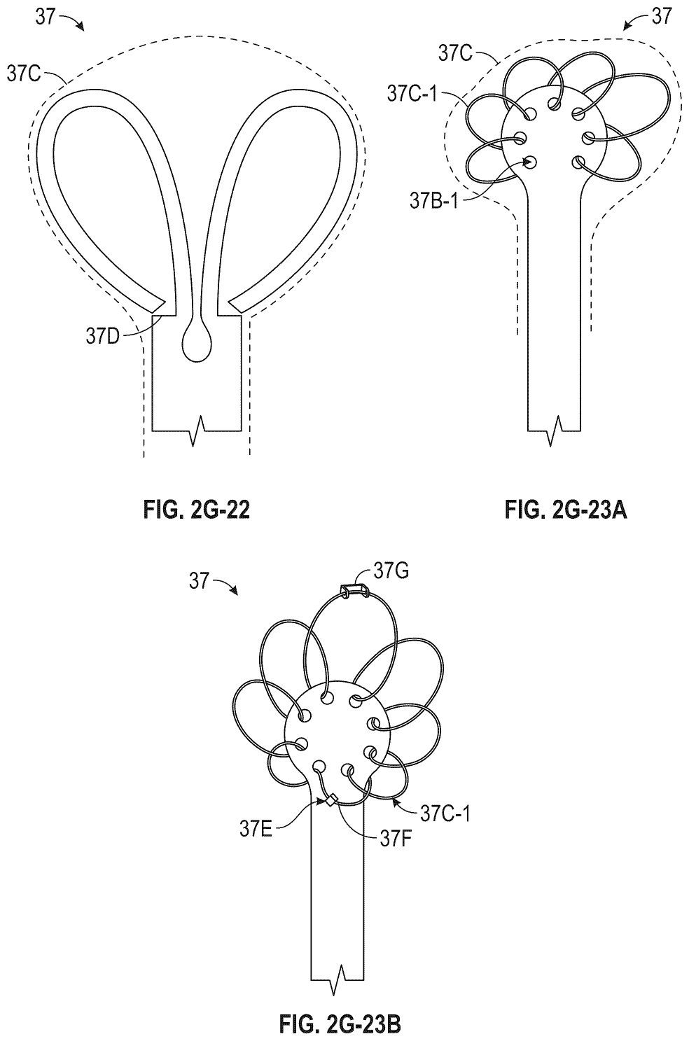

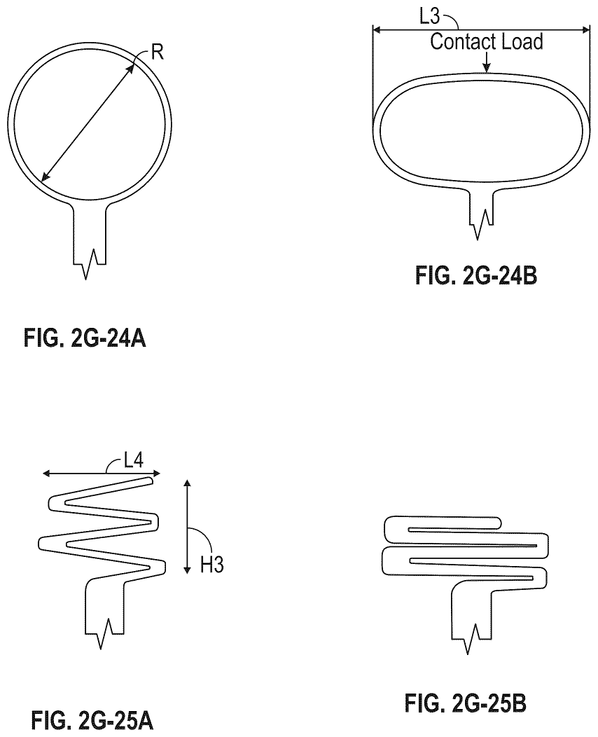

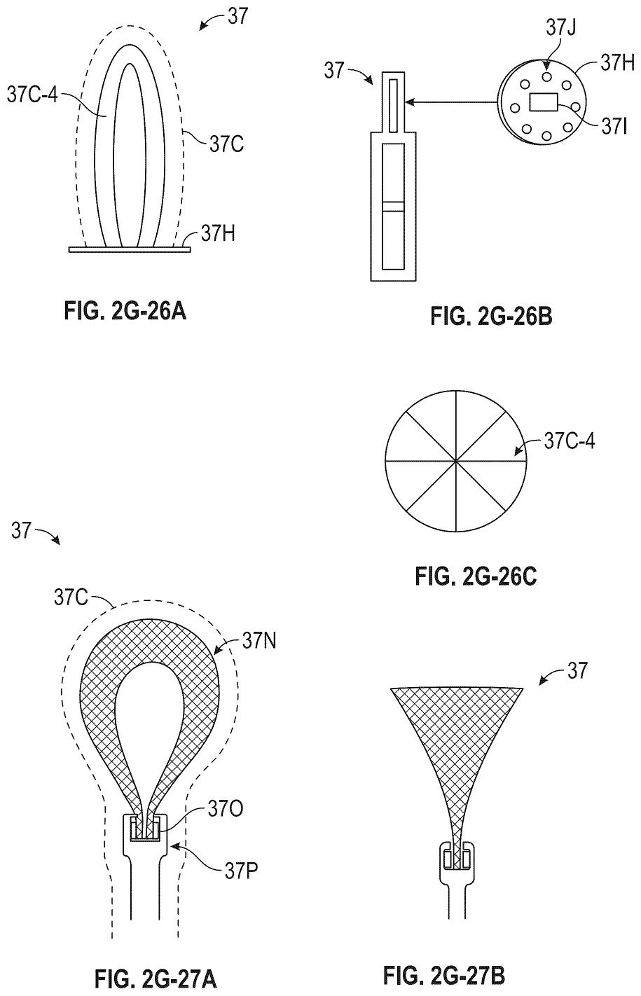

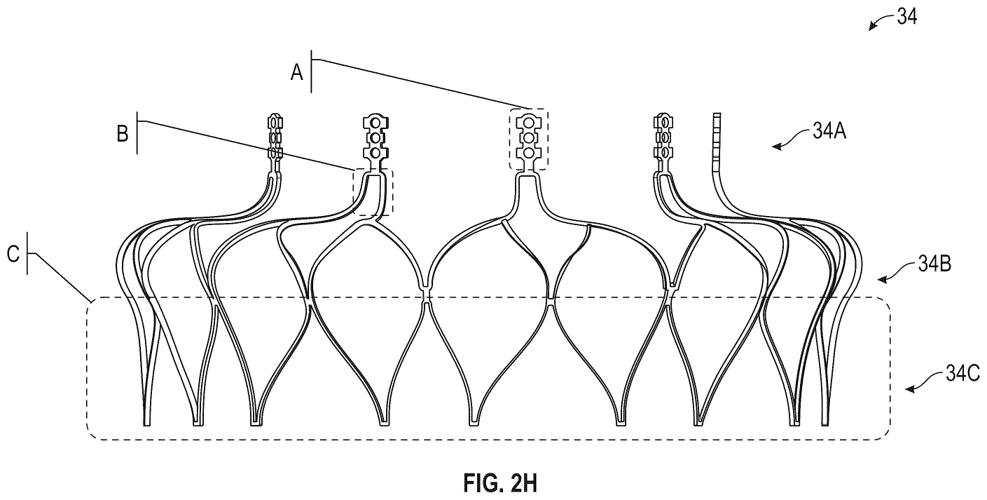

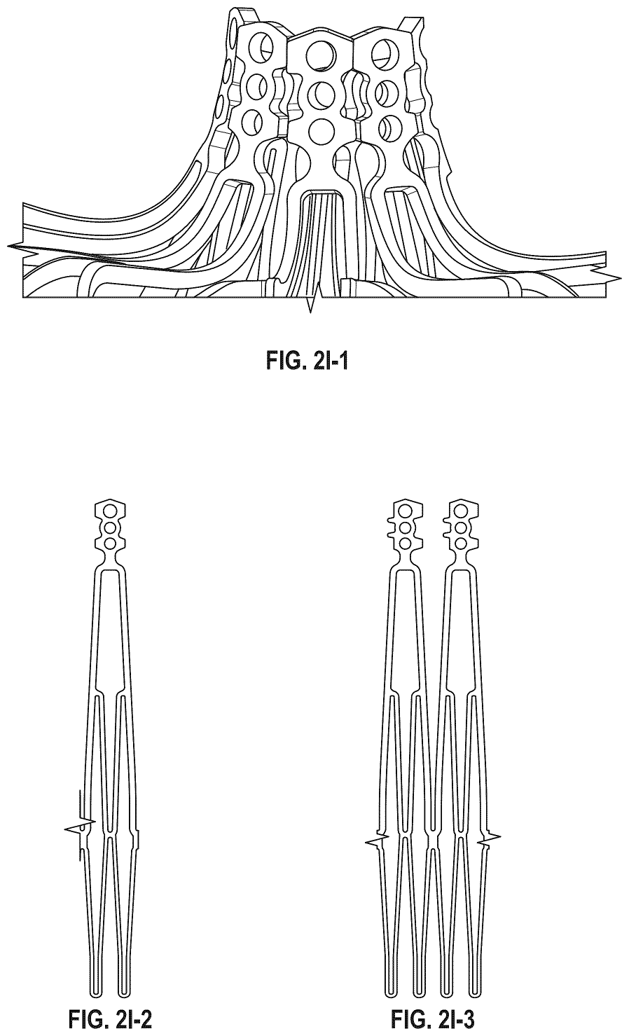

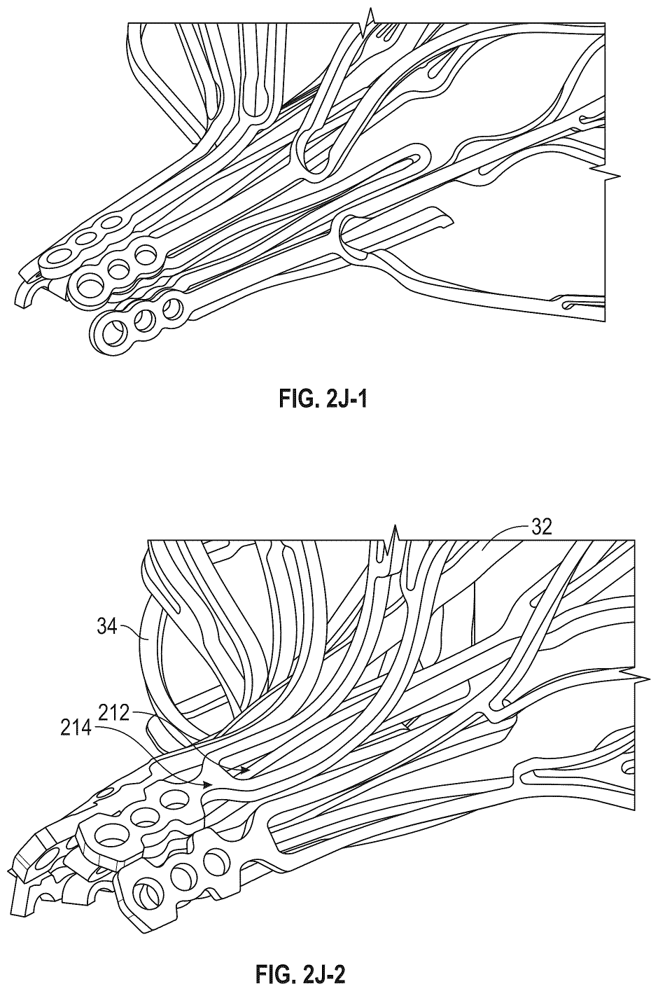

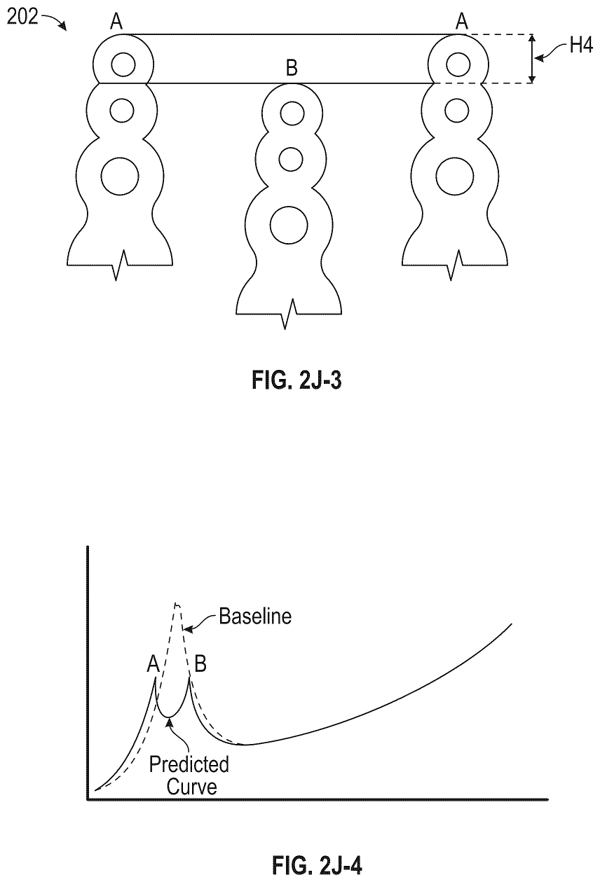

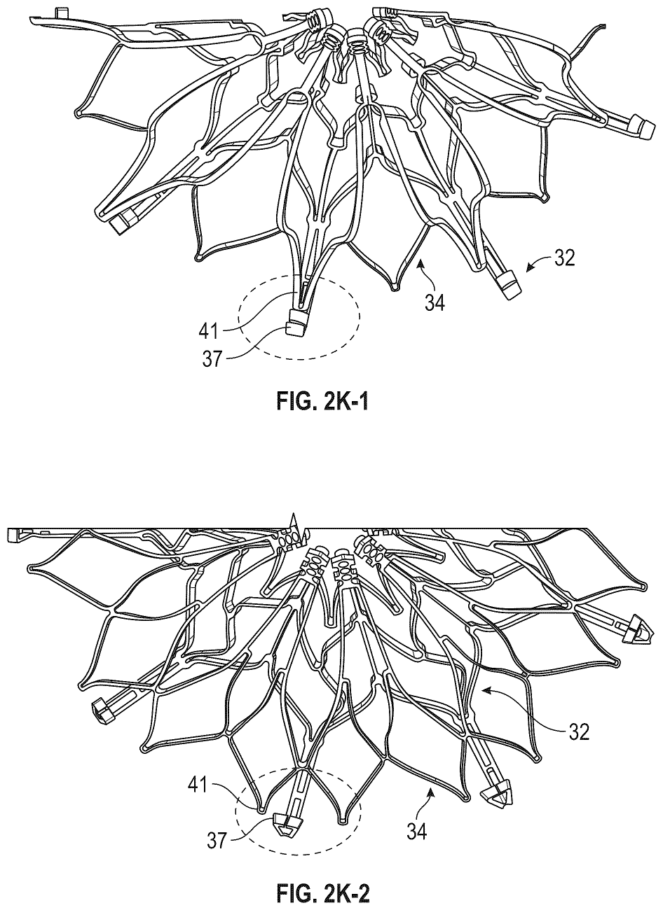

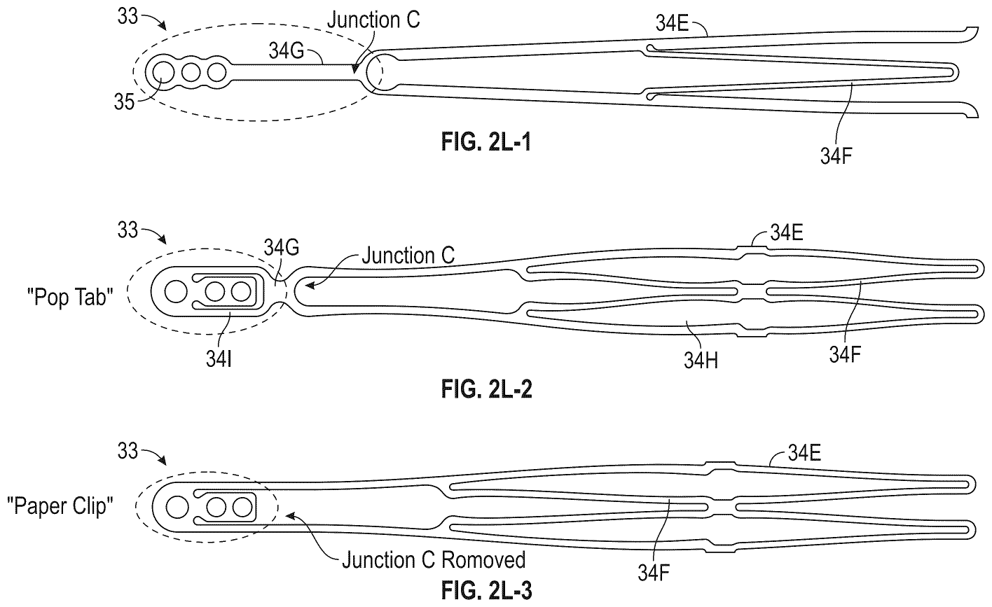

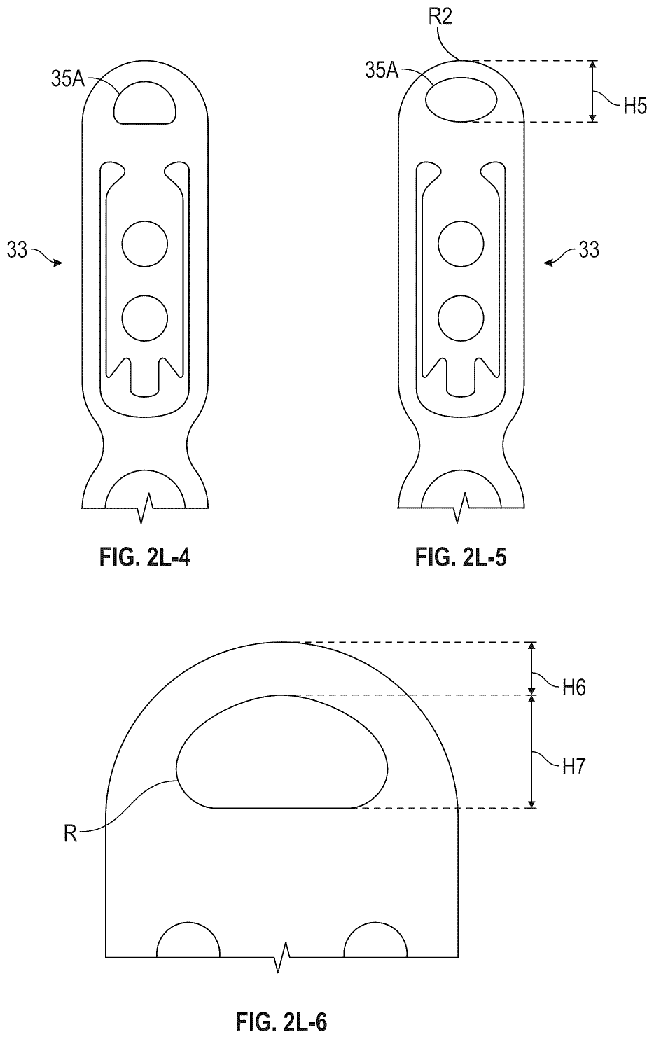

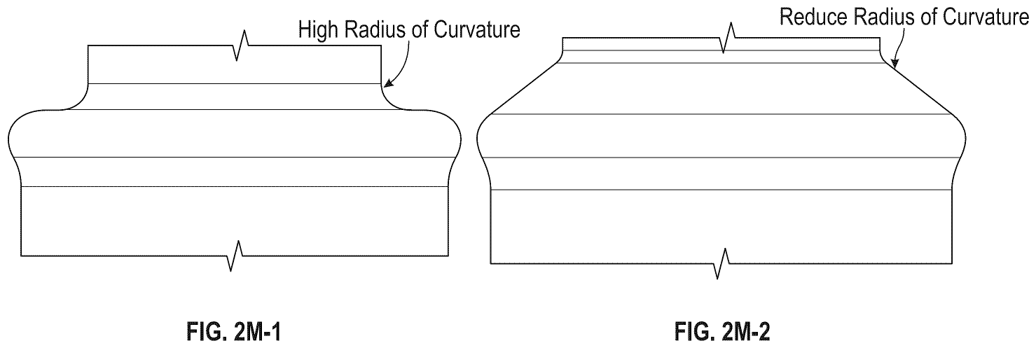

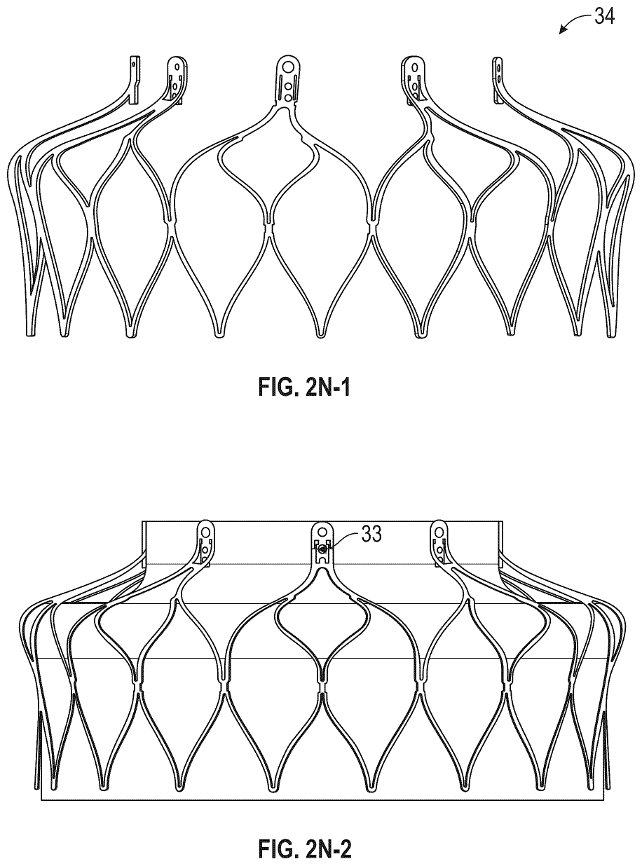

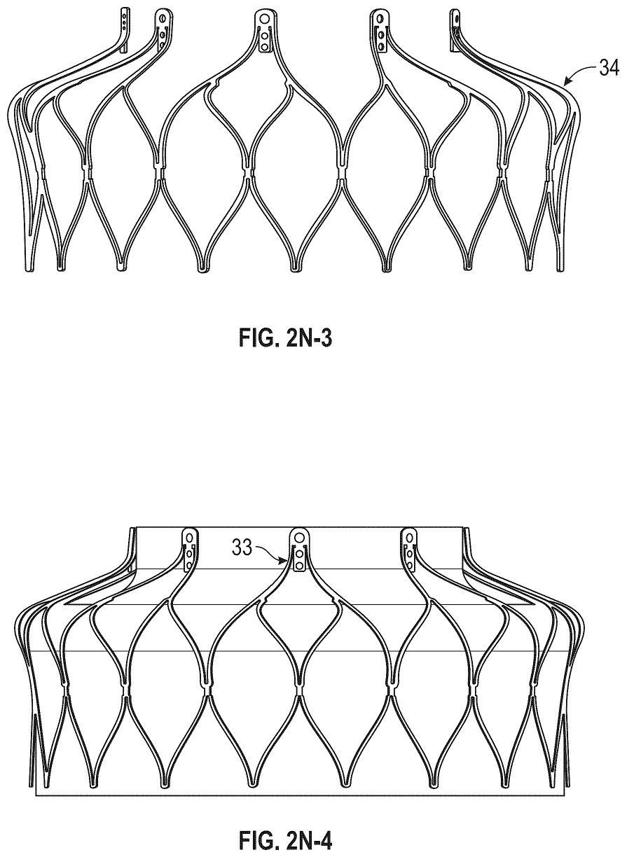

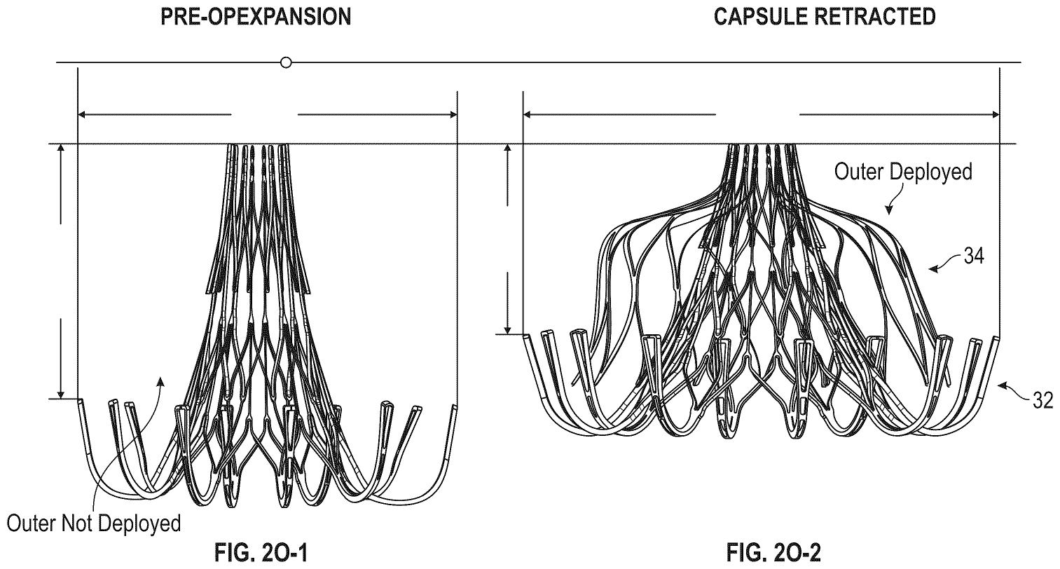

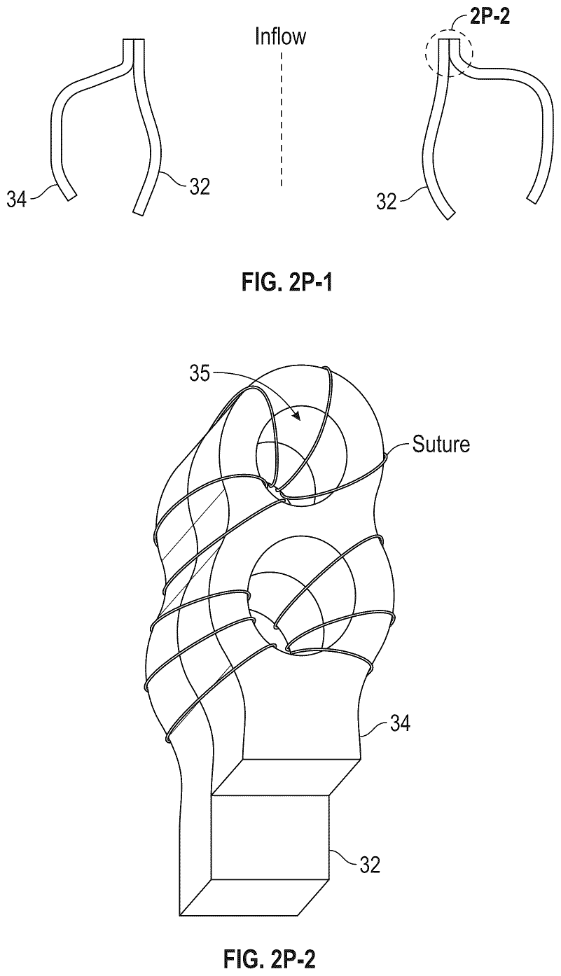

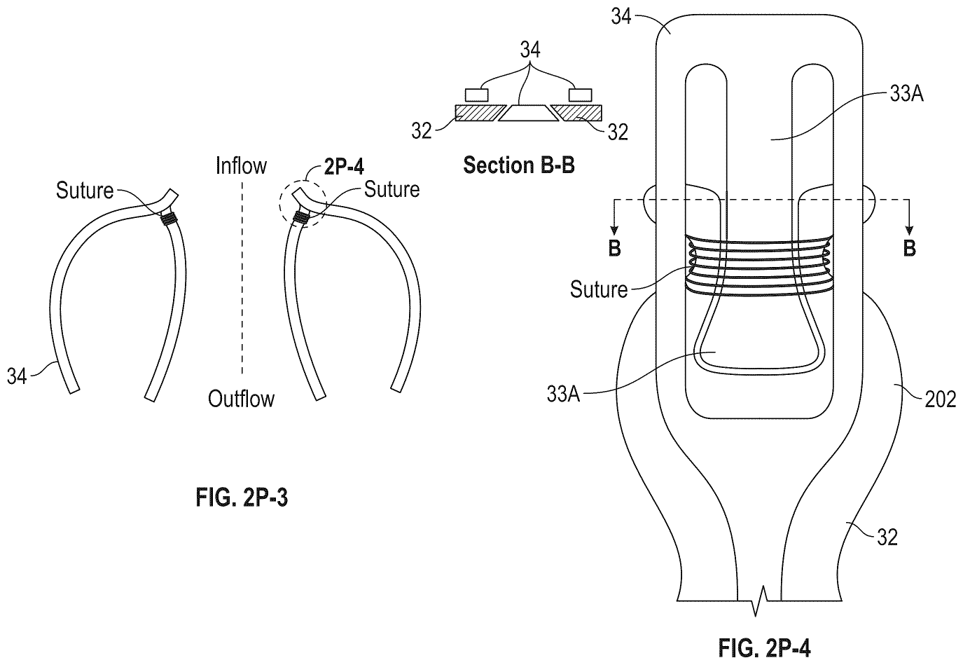

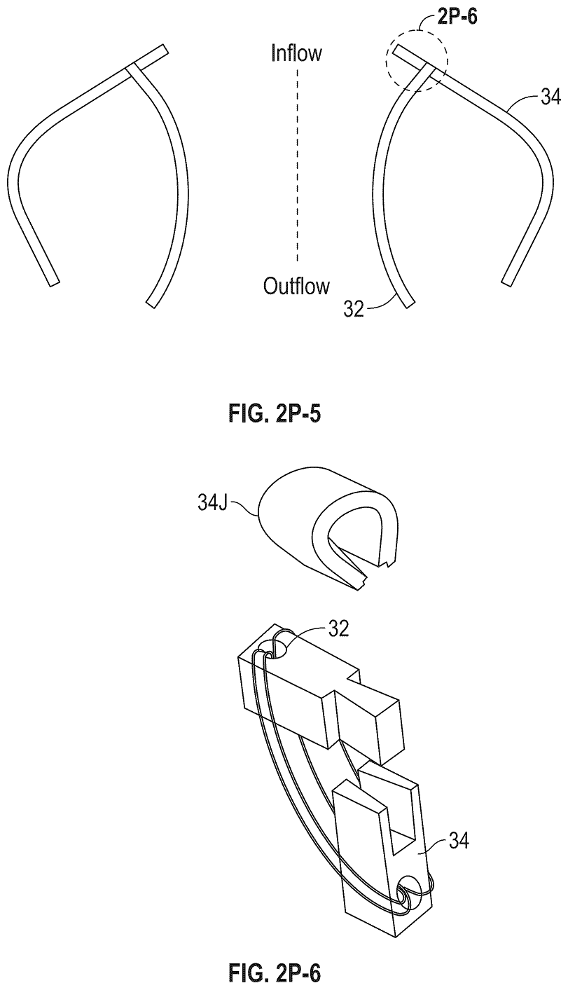

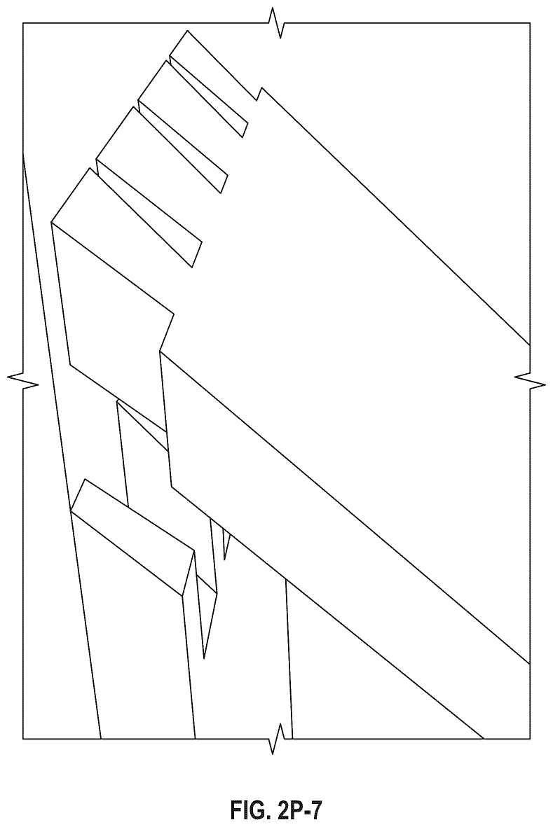

FIG. 2 shows an example frame structure for an implant (e.g., valve prosthesis) 30 that can be pre-loaded into and delivered by the delivery device 15. The implant 30 includes a dual frame assembly including an inner frame 32 and an outer frame 34 that are aligned and coupled together during manufacture. FIG. 2A illustrates an embodiment of the inner frame 32. The inner frame 32 can include a proximal, or inflow, portion 32A, a middle, or intermediate, portion 32B, and a distal, or outflow, portion 32C. The inner frame 32 can be shaped to exhibit a generally hourglass shape in an expanded configuration, in which the middle portion 32B has a smaller cross-sectional width than the cross-sectional width of the proximal portion 32A and the distal portion 32C. The proximal portion 32A may include tabs 33 and/or eyelets 35 to facilitate engagement with other structures or materials (e.g., the outer frame 34, a skirt or fabric assembly, a prosthetic valve assembly, and/or tethers or retention sutures of the delivery device 15). The distal portion 32C may include outwardly and upwardly-extending anchors 37 to facilitate anchoring at a desired target location (e.g., a native heart annulus). The inner frame 32 may have a chevron cell structure as shown in FIG. 2A. However, other cell structures may be used. The inner frame 32 may include a prosthetic valve assembly coupled thereto comprising a plurality of prosthetic valve leaflets (not shown). FIG. 2B illustrates an embodiment of the outer frame 34. The outer frame 34 may also include a proximal, or inflow, portion 34A, a middle, or intermediate, portion 34B, and a distal, or outlet, portion 34C. Similar to the proximal portion 32A of the inner frame 32, the proximal portion 34A of the outer frame 34 may also include one or more eyelets 35 to facilitate coupling to one or more structures or materials (e.g., the inner frame 32, a skirt or fabric assembly, and/or to tethers or retention sutures of the delivery device 15). For ease of understanding, in FIGS. 2, 2A, 2B, the prosthesis 30 is shown with only the bare metal frame structures illustrated. FIG. 2C illustrates an embodiment of a fully-assembled implant (e.g., valve prosthesis) 30 including a skirt assembly 38 positioned between the frames 32, 34 and padding 39 surrounding the anchors 37. The implant (e.g., prosthesis) 30 can take any number of different forms or designs.

Additional details and example designs for an implant (e.g., prosthesis or replacement heart valve) are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, 2018/0055629 and 2019/0262129 (e.g., hourglass shape of inner frame). The entirety of these patents and publications are hereby incorporated by reference and made a part of this specification. Further details and embodiments of a replacement heart valve or prosthesis and its method of implantation are described in U.S. Publication Nos. 2015/0328000, 2016/0317301, 2019/0008640, and 2019/0262129, the entirety of each of which is hereby incorporated by reference and made a part of this specification.

### Frame Structural Features

FIGS. 2D-1 to 2D-3 illustrate how structural instability (e.g., strut buckling) can occur during compression (e.g., crimping, mid-loading) of a standard chevron-cell frame structure. When a chevron-cell frame is progressively reduced in diameter (e.g., funneled), such as when a frame is loaded into a shaft assembly of a delivery device having a smaller diameter than the frame in the expanded configuration, structural instability (e.g., ovality) of the cells and struts of the chevron-cell frame can occur. This structural instability can hamper an implantation procedure and, in extreme cases, can reduce structural integrity of the frame. The structural instability can produce unpredicted stress or strain on the frame, which could compromise durability, leading to device failure. With reference to FIG. 2D-1, the chevron-cell structure, as it is crimped or funneled, drives internal forces through its constituent struts. When a chevron-cell frame is partially funneled or crimped, the internal forces are at a maximum, with some cells partially open and others partially closed. A conventional chevron-cell structure can become an inherently unstable system, wherein the portion or section of the frame that is undergoing reduction in diameter begins to forelengthen. Forelengthen may be the converse of foreshorten. In some implementations, forelengthen may mean the same as lengthen. The portion or section of the frame that is still fully expanded resists the forelengthening, and strut buckling can occur as a result. When partially funneled, axial beam or strut 202, for example, of the fully expanded portion of the frame can buckle in an unpredictable direction, which can lead to ovality cascade, as shown in FIG. 2D-2 (bottom view of a partially-funneled, or partially-crimped, inner frame with a conventional chevron-cell structure) and FIG. 2D-3 (side perspective view of a partially-funneled, or partially-crimped, inner frame with a conventional chevron-cell structure). When partially funneled, axial beam or strut 202 may be under compression and axial beams or struts 203, 204 may be under tension.

FIGS. 2E-1 to 2E-4, 2F-1 and 2F-2, and 2G-1 to 2G-3 illustrate various views of embodiments of inner frames having a chevron-cell structure that include structural mechanisms or features configured to dynamically absorb, or compensate for, the forelengthening of the partially-crimped section of the inner frame. The structural mechanisms are designed to be able to compress or expand in a controlled manner, thereby changing the frame from an unstable system during loading or deployment into a stable system. In several embodiments, the structural mechanisms are design to compensate for internal compression forces on slotted strut members and provide dynamic frame stability, thereby ensuring improved frame integrity and patient safety. In several embodiments, the structural mechanisms provide frame stability by increasing lateral and/or circumferential bending stiffness similar to that of a diamond cell structure but without increasing crimp length as a diamond cell structure would. In several embodiments, the structural mechanisms advantageously prevent, or reduce the likelihood of, oval loading and deployment (e.g., by creating radially non-uniform, out-of-plane expansion of slotted strut members (e.g., axial beams or struts).

In accordance with several embodiments, an expandable and compressible frame can include a plurality of structural mechanisms (e.g., axial (longitudinal) connecting portions, such as strut components, within one or more chevron or diamond-shaped cells of a distal or outflow end portion of the expandable frame) that are capable or reducing in length (e.g., foreshortening) in a predictable manner. The structural mechanisms are configured to cause at least a portion of the frame (e.g., certain cells or struts) to buckle, deform, or bend in a predictable manner or in a desired direction (such as when the frame is being compressed in a non-uniform manner (e.g., a portion of the frame is being compressed while another portion remains expanded) through a funnel-shaped loader or when the frame is being compressed in a non-uniform manner as it is being recaptured within a delivery device). The structural mechanisms may comp[rise bendable axial struts that can shorten and accommodate the temporary non-uniform shape. Although the structural mechanisms may only be included in some of the cells of the frame, the predictable bending may cause adjacent cells or portions to also bend or crimp in a similar manner, thereby providing controlled bending, and compression, of the frame. In some configurations, the structural mechanisms may be biased in a particular configuration or shape so as to bend, deform, or crimp in a desired direction.

In some configurations, an implant (e.g., replacement heart valve) includes a self-expandable frame configured to transition between a compressed configuration and an expanded configuration. The frame includes a plurality of rows of cells (e.g., chevron-shaped cells) formed by cell struts. At least one cell of a distal-most row of the plurality of rows of cells includes a structural component that is adapted to prevent cell ovality during transition between the compressed configuration and the expanded configuration, and vice-versa. The structural component may include, for example, an axial strut connecting a distal apex of the at least one cell with a distal apex of a bordering cell in a row immediately above the distal-most row. Rows other than the distal-most row may include the structural component in addition to or as an alternative to the distal-most row.

FIGS. 2E-1 to 2E-4 illustrate an embodiment of an inner frame 32 having axially asymmetric “bow spring” structural mechanisms. FIG. 2E-1 shows the inner frame 32 in a crimped configuration and FIG. 2E-2 shows the inner frame 32 in an expanded configuration. The bow spring structural mechanisms are built into one or more of the axial struts 202 extending between the chevron cells. FIG. 2E-3 shows a side perspective view of the inner frame 32 in a partially-crimped, or partially-compressed configuration in which a proximal, or inflow portion, 32A of the inner frame 32 is crimped or compressed but the distal, or outflow portion, 32C, of the inner frame 32 is still fully expanded. With reference to FIG. 2E-3, the V-shaped struts forming the top boundaries of at least the distal-most row or ring of cells fold up or compress prior to the V-shaped struts forming the lower boundaries of the distal-most row or ring of cells. Therefore, the distance between the endpoints of the bowspring axial struts 202 shortens during crimping. The bowspring axial struts could be removed but this could result in the frame being more flimsy. FIG. 2E-4 shows a top view of FIG. 2E-3 with the inner frame 32 in the same configuration. As shown in FIGS. 2E-3 and 2E-4, the bowspring axial struts 202 are designed to dynamically compensate for compression during device loading so as to avoid ovality. The bowspring axial struts 202 deform in a stable and predictable manner. The bowspring axial struts 202 may advantageously not elongate when crimped such that the frame crimp length does not increase during loading or deployment. The laser cut pattern of the bowspring axial struts 202 may comprise a narrow slot to facilitate non-lengthening (e.g., no forelengthening) of the frame during loading, deployment, and/or recapture. The bowspring axial struts 202 can be created at an angle less than perpendicular or perpendicular to a long axis of the frame 32, as desired and/or required. The performance of the bowspring feature (e.g., bowspring axial struts 202) is governed by the geometry of the intended bending region. Within this bending region, the length, wall thickness, strut width, lasercut arc, and/or taper region directly affect the degree of bending and strain experienced by the material. The embodiment shown in FIGS. 2E-1 to 2E-4 depicts a bowspring axial strut 202 wherein the intended bending region has a tapered strut width, which reduces to a minimum at the midpoint of the bowspring arc, and an arched shape, generated by the lasercut pattern, which predisposes the intended bending region to bend in the desired direction. The ratio of the bowspring features' wall thickness to strut width ensures the bending is predictable and mostly unidirectional. In some implementations, the length of the bowspring axial strut 202 is tailored to ensure the required compressive travel is within material limits.

The bowspring embodiment in FIGS. 2E-1 to 2E-4 demonstrates a mechanism to compensate for frame forelengthening under compression, wherein the bowspring axial struts 202 dynamically reduce in length. The bowspring axial struts 202 in FIGS. 2E-1 to 2E-4 comprise single curved struts that bow to one side in a predictable manner. As can be seen in the transition between FIGS. 2E-2 and 2E-4, the bend in the bowspring axial struts 202 becomes more pronounced and the bends all bow in a uniform, single direction. The principles of the mechanism work the same in reverse, wherein a pre-shaped bowspring mechanism under tension could dynamically elongate to compensate for the progressive forelengthening of the chevron-style frame design as it is loaded/deployed from its delivery device or system.

The bowspring mechanism (e.g., bowspring axial struts 202) may be suitable for frames constructed of nitinol or any other super-elastic shape-memory alloy. This mechanism may also be employed for frames comprised of steel, cobalt-chromium, or other alloys, ensuring a conically crimped implant remains circular as it is diametrically reduced along its length. Use of this design in a frame made of these materials would remain deformed and be beneficial for use in applications where forcing local regions of a frame radially inward or outward is desired, such as to generate an hourglass shape (inward) or anchoring protrusions (outward).

The ability of the axial strut (e.g., bowspring axial strut 202), the part of the unstable chevron cell structure under compression, to dynamically reduce in length during device (e.g., implant) loading, can be achieved by via number of different mechanisms, of which the bowspring concept is one. Another mechanism to achieve dynamic length change is to seed the axial beam with a multitude of latitudinal lasercut windows that could close or open to balance the compressive forces exerted on the strut during loading. Another mechanism to achieve dynamic length change of the axial beam is to build in a slot and pin mechanism, wherein the proximal section of the axial beam or strut terminates in a pin which engages a slot in the distal section of the axial beam or strut. As the frame is loaded, the pin can translate along the slot, thereby balancing the forelengthening of the chevron design, and when fully expanded and experiencing anatomical forces, the pin can lock to ensure a dependable frame structure.

The degree of axial asymmetry may vary. FIG. 2F-1 illustrates an embodiment of an inner frame 32 having highly asymmetric “bow spring” structural mechanisms and FIG. 2F-2 illustrates an embodiment of an inner frame 32 having minimally asymmetric “bow spring” structural mechanisms. The bowspring structural mechanisms may also be axially symmetric. FIGS. 2G-1, 2G-2, and 2G-3 show various views of an embodiment of an inner frame 32 having symmetric dual “bow spring” structural mechanisms. The dual bow spring structural mechanisms comprises a pair of struts that bow to opposite sides similar to how a coin purse functions. FIG. 2G-1 shows a close-up view of one symmetric dual “bow spring” structural mechanism while the inner frame is in a crimped or compressed configuration. FIG. 2G-2 shows the inner frame 32 in an expanded configuration. FIG. 2G-3 shows the inner frame 32 in a partially-crimped, or partially-compressed configuration in which a proximal, or inflow portion, 32A of the inner frame 32 is crimped or compressed but the distal, or outflow portion, 32C, of the inner frame 32 is still fully expanded. If a frame has a curved profile in the region of interest, as is the case with the hourglass profile of the inner frames 32 described herein, the out-of-plane frame expansion may convert the slot within the chevron cell into a dual bow spring mechanism. The dual bow spring mechanism converts compressive loads that, if left unchecked or uncompensated for would lead to uncontrolled buckling, into a controlled bending of the bow spring struts.

### Anchor Features

In accordance with several embodiments, the anchors 37 of an expandable frame (e.g., the inner frame 32 of a dual-frame replacement heart valve) may be formed without the use of foam cushions on the anchor tips that contact native heart tissue. The anchors may include non-foam and/or non-fabric dampeners made from flexible material (e.g., metal or metal alloy material) that is attached to an anchor tip that can be bent, deformed, or contoured to provide a cushioning effect. In some embodiments, the dampeners or anchor tips are designed to be “softer”, or more cushioned, in one direction to reduce conduction disturbances (e.g., conduction disturbances caused by pressure applied to a septal wall by a rigid anchor tip portion) and more rigid in the other opposite direction to preserve capture of native valve leaflets. The anchor tips may also have reduced anchor profiles to facilitate easier procedural navigation and placement of the replacement heart valve. The anchor tips may be further designed so as not to puncture heart anatomy (e.g., no sharp edges and provide a cushioning effect). The anchor tips may additionally be designed to reduce loading forces in the catheter or to make the loading forces more predictable.

FIGS. 2G-4A, 2G-4B, 2G-5, 2G-6, 2G-7, 2G-8, 2G-9A, 2G-9B, 2G-10, 2G-11A, 2G-11B, 2G-11C, 2G-12, 2G-13, 2G-14, 2G-15, 2G-16, and 2G-17 illustrate various views of embodiments of atraumatic anchor tips of an expandable frame of a replacement heart valve. In particular, FIGS. 2G-4A, 2G-4B, 2G-5 and 2G-6, illustrate embodiments of an attachable tip, or attachable anchor dampener, 37A, and FIGS. 2G-7, 2G-8, 2G-9A, 2G-9B, 2G-10, 2G-11A, 2G-11B, 2G-11C, 2G-12, 2G-13, 2G-14, 2G-15, 2G-16, and 2G-17 illustrate other embodiments of an attachable anchor tip, or padded tip, 37B. The embodiments of FIGS. 2G-4A, 2G-4B, 2G-5, 2G-6, 2G-7, 2G-8, 2G-9A, 2G-9B, 2G-10, 2G-11A, 2G-11B, 2G-11C, 2G-12, 2G-13, 2G-14, 2G-15, 2G-16, and 2G-17 may not incorporate the use of foam padding and may or may not incorporate the use of a cloth covering. Thus, a cloth covering may be optional in accordance with these embodiments. The anchor tips may be incorporated into all, some, or one of the anchors.

In more detail, FIGS. 2G-4A and 2G-4B illustrate one embodiment of an attachable anchor tip or dampener 37A which can be attached to an anchor 37 of inner frame 32 of a dual-frame valve prosthesis. The dampener 37A may be a single, thin polymeric (e.g., plastic or elastomeric) or metal strip (e.g., or other material flexible enough to be easily bent). For instance, the dampener 37A of FIG. 2G-4B has a thin strip shape that is bent over the distal tip (e.g., upwardly-extending tip when in an expanded configuration) of the anchor 37 to form a saddle-like design. In some configurations, the dampener 37A is formed of a flat raw material (e.g., a thin metal material). Alternatively, the dampener 37A may be formed from tubing, may be 3D printed, and/or may be formed of wire material. The material may include but is not limited to nitinol, cobalt chrome, stainless steel, or polymer material. As the dampener 37A contacts anatomical tissue, a radius of the bent loop portion increases due to the flexibility of the material, thereby resulting in a “cushioning” effect. The dampener 37A may be adhered to the anchor 37 via adhesive, welding, suture, or other attachment mechanism. For example, the dampener 37A can be tied to the anchor 37 using threads or wires inserted through one or more suture holes 37A-1 formed on the end portions of the dampener 37A. Different shapes or designs can be implemented. For example, FIG. 2G-5 illustrates another embodiment of a dampener 37A which has a plurality of slits 37A-3 so as to reduce vibration when there is an external impact on the dampener 37A. The plurality of slits 37A-3 may also cause a fanning out of the contact surface to increase surface area. Such a dampener 37A can also provide a cushioning effect while protecting the tip of the anchor 37. The dampener 37A can be tied to the anchor 37 of inner frames 32 as shown in FIG. 2G-6 by suturing around end portions 37A-4 of the dampener 37A using threads or wires 37A-2 wrapped around the end portions 37A-4 and/or inserted through one or more suture holes 37A-1 formed on the end portions 37A-4 of the dampener 37A.

FIGS. 2G-7, 2G-8, 2G-9A, 2G-9B, 2G-10, 2G-11A, 2G-11B, 2G-1C, 2G-12, 2G-13, 2G-14, 2G-15, 2G-16, and 2G-17 also illustrate embodiments of attachable anchor tips similar to those illustrated in FIGS. 2G-4A, 2G-4B, 2G-5 and 2G-6 except that the attachable tips in FIGS. 2G-7, 2G-8, 2G-9A, 2G-9B, 2G-10, 2G-11A, 2G-11B, 2G-1C, 2G-12, 2G-13, 2G-14, 2G-15, 2G-16, and 2G-17 may be made of a flat/thin raw material or a thicker rigid material. For instance, FIG. 2G-7 shows a tube-shaped attachable tip 37B that may have horizontally-formed slits 37B-3A at one side (e.g., front side) which allow inward flexion while preventing outward flexion. The slit cuts 37B-3A may help to maintain rigidity for leaflet capture. The tube-shaped attachable tip 37B further includes open cuts 37B-3B on the opposite side (e.g., radially inward side facing the inner frame 32) which allow inward flexion. The slits 37B-3A and the open cuts 37B-3B can be formed, for example, by laser cutting a flexible hypotube. The tube-shaped attachable tip 37B can distribute and dampen loads and reduce force applied inside the patient's body, and further the slits 37B-3A can maintain for rigidity for leaflet capture. An optional padded anchor tip can be attached to a top of the tube to distribute and dampen load.

FIG. 2G-8 shows a double half loop attachable tip 37B design that includes an outer half loop 37B-4A (the loop that is farther from the inner frame 32) and an inner half loop 37B-4B (the loop closer to the inner frame 32) that provide asymmetric stiffness. The half loop shapes may advantageously facilitate distributing of load. The inner half loop 37B-4B may be thicker than the outer half loop 37B-4A and thus more rigid for maintaining reliable leaflet capture. The outer half loop 37B-4A may optionally incorporate a plurality of relief cuts 37B-3C. The outer half loop 37B-4A is designed to provide a cushion effect to aid in reducing conduction disturbances and in decreasing the amount of force applied to the anatomy (e.g., septum, annulus). Similar to other attachable tip, the double half loop attachable tip 37B may have one or more suture holes 37B-1A for attaching the half loops to the inner frame 32 or anchor tip 37 by suturing or other attachment method. In addition, the double half loop attachable tip 37B may have upper suture holes 37B-1C and lower suture holes 37B-1B for suturing the outer half loop 37B-4A and the inner half loop 37B-4B together. The half loops may be laser cut from a flat sheet or tube (may be the same tube or different tubes of different thickness so that the inner tube is thicker) and shape set to the same shape using the same tooling. One or both of the half loops may optionally be covered with a sleeve (e.g., cloth sleeve).

FIGS. 2G-9A and 2G-9B show a side view and a front view, respectively, of another embodiment of an attachable anchor tip 37B that includes a half loop that terminates in a flexible spring-shaped end. The attachable anchor tip 37B of FIGS. 2G-9A and 2G-9B may be rigidly and fixedly attached to the anchor 37 by suturing one end having suture holes 37B-1 to the anchor 37 while the opposite end (e.g., spring-shaped end) thereof may remain free and unattached. The spring-shaped end of the half loop attachable tip 37B may allow the whole anchor to deflect off of sensitive anatomy (e.g., septal wall), thereby providing a cushioning effect, reducing force applied to the anatomy along the conduction pathway, and reducing conduction disturbances. The entire anchor tip design may be laser cut from a flat sheet and then the half loop portion can be shape set into a half loop shape without requiring the spring-shaped end to be shape set. FIG. 2G-10 shows an anchor tip loop attachable tip 37B similar to the embodiment of FIGS. 2G-4A and 2G-4B, but further includes a wire 37C-1 wrapped over at least a portion of the loop to provide further springy and cushiony effects. The wire may only extend along an outer side and top of the loop (e.g., side configured to contact a septal wall or annulus) and not along the entire loop. The wire 37C-1 allows the anchor tip to deflect off of sensitive anatomy as opposed to pressing rigidly into it. On the inward side (e.g., leaflet side) of the loop, there may be no wire wrap in order to preserve leaflet capture ability. The attachable tip 37B of FIG. 2G-10 can be also made by laser cutting a flat sheet to have a loop shape and the wire 37C-1 can be wrapped through holes cut through a thickness of the loop. The ends of the loop may be sutured to the anchor 37 via suture holes 37B-1 or other attachment mechanisms as described previously.

FIGS. 2G-11A, 2G-11B, 2G-11C, 2G-12, 2G-13, 2G-14, and 2G-15 illustrate other embodiments of an attachable tip 37B of one or more anchors of an inner frame. The attachable tips shown in these embodiments may have more than two arms. For example, referring to FIGS. 2G-11A and 2G-11C, the attachable tip 37B may include first opposite arms 37C-2 having suture holes 37B-1 for attachment to the anchor at each end thereof, and second opposite arms 37C-3 having arms of a generally continuous width and having free, unattached ends. The attachable tip 37B can be formed of a wire, a thin metal, or any flexible polymeric or metallic material to bend over the anchor distal tip as shown in FIG. 2G-11C, and the first opposite arms 37C-2 can be attached to the anchor 37 by suturing sutures or threads 37B-2 through the suture holes 37B-1 while the second opposite arms 37C-3 may be free at their ends as shown in FIG. 2G-11B. FIGS. 2G-12 to 2G-15 illustrate various embodiments of attachable tip designs similar to FIG. 2G-11A. That is, an attachable tip 37B of FIG. 2G-12 may have circular ends at the second opposite arms 37C-3, and an attachable tip 37B of FIG. 2G-13 may be similar to that of FIG. 2G-12 but may have a circular shape at the center with a center hole 37B-4 forming a larger surface contact with the anatomy. FIGS. 2G-14 and 2G-15 are variants of FIGS. 2G-12 and 2G-13, respectively, with more than two second opposite arms 37C-3. The number of free, unattached arms may vary.

FIGS. 2G-16 and 2G-17 illustrate an attachable tip for attaching to the end of the inner frame 32 or the anchor 37 similar to the above-described embodiments. The attachable tip of FIGS. 2G-16 and 2G-17 has a symmetric configuration so that they can be folded so that the upper and lower tips can be in contact and attached to the inner frame 32 by suturing through suture holes 37B-1.

FIGS. 2G-18A, 2G-18B, 2G-19, 2G-20A, 2G-20B, 2G-21A, 2G-21B, 2G-22, 2G-23A, 2G-23B, 2G-24A, 2G-24B, 2G-25A, 2G-25B, 2G-26A, 2G-26B, 2G-26C, 2G-27A and 2G-27B illustrate various embodiments of anchor tips for anchors designed to capture native heart valve leaflets (e.g., native leaflets of the mitral or tricuspid valve). The anchor tip configurations may advantageously provide a cushioning function without use of, or a reduction in an amount of, foam or cloth components. In accordance with several embodiments, the anchor tips represent modifications to existing frame material (e.g., modifications to on, some or all, of the anchors of the frame themselves) instead of attachments to the anchors, such as embodiments described previously. The anchor tip designs may be incorporated into one, some, or all of the anchors of a frame. In some implementations, the anchor tips comprise non-fabric and/or non-foam anchor tips made from a flexible material (e.g., metal or metal alloy material such as nitinol) that can be bent, contoured, or depressed to provide a cushioning effect on at least a portion of the anchor tip.

FIGS. 2G-18A and 2G-18B illustrate a dual-layer hoop anchor forming two independent hoops stacked on top of one another. The hoops may be cut from the anchor tube stock and then shape set to separate the independent hoops out of plane to double the contact surface area (as shown best in FIG. 2G-18B). FIG. 2G-19 illustrates a dual inward spiral anchor formed by two independent spirals positioned side by side that may be formed by cutting an anchor tube stock. The spirals can deflect to provide a cushioning effect. This anchor design may not require any shape setting or welding. A thickness D of the spirals of the dual inward spiral anchor may be, for example, from 100 μm to 200 μm.

FIGS. 2G-20A and 20B each illustrate a heart-shaped hoop anchor 37 formed by a single hoop with two lobes such that the center of the heart shape can deflect to cushion the anchor load. In particular, FIG. 2G-20A may have a length L which narrows to slip past chordae tendineae and a height H1 which deflects to reduce impact loading and wear on a leaflet or annulus as a shock absorber. In addition, the heart shaped hoop anchor 37 of FIG. 2G-20A may have a sleeve or cloth sock 37C around the anchor 37. The heart shaped hoop anchor 37 of FIG. 2G-20B may optionally have a snap configuration where a top member 37CC of the hoop snaps into a base 37CD of the hoop for shape setting and/or for reducing a crimped length (e.g., by several millimeters). Upon uncrimping, such a hoop snap becomes free. The heart shaped hoop anchor design of either FIG. 2G-20A or 20B may not require any shape setting or welding.

FIGS. 2G-21A and 2G-21B each illustrates a bunny ear cushion anchor configuration formed by two outward facing spirals next to each other which bend and separate upon loading to distribute the load and cushion the anchor contact with the heart anatomy. FIG. 2G-21A illustrates a narrower (e.g., L1 is about 2 mm while L2 is about 6-7 mm) and taller (e.g., H2 is 3-4 mm) anchor profile compared to FIG. 2G-21B, thereby allowing easier slip through or removal from chords. On the other hand, the wider version of FIG. 2G-21B may allow a wider, more distributed load when the anchor 37 or the inner frame 32 is positioned against the native valve annulus or leaflet. One or both of the spirals may optionally be covered with cloth sleeves to facilitate spreading. No shape setting or welding may be required.

FIG. 2G-22 illustrates a collapsible loop cushion anchor design formed by two outward facing loops similar to the embodiment of FIG. 2G-21A with additional support from a ledge 37D that creates a stiffer (e.g., more rigid) loop when contacting from the distal end and a softer loop while contacting from the proximal end, thereby allowing easier disengagement from interaction with chordae tendineae anatomy when pulling out the valve prosthesis. The anchor may optionally be covered with a cloth sleeve or sock 37C.

FIGS. 2G-23A and 2G-23B each illustrates a wire wrap anchor tip design where the anchor has a plurality of holes 37B-1 (e.g., laser cut holes) through which a wire 37C-1 can be wrapped loosely, creating a soft “cushioned” tip of the anchor 37. In particular, FIG. 2G-23A may optionally include sleeve or cloth sock 37C covering the wire 37C-1 and wire ends 37F may be welded or crimped as a stopper 37E, and FIG. 2G-23B may include a radiopaque marker 37G to indicate deflection from annulus contact. Wire ends 37F of FIG. 2G-23B may be welded together. The wires 37C-1 of FIGS. 2G-23A and 2G-23B may be made of nitinol, cobalt chrome, stainless steel, polymer, radiopaque metal, or the like. This anchor tip design may not require any shape setting.

FIGS. 2G-24A and 2G-24B illustrate an anchor having a thin-walled hoop cut into an end of the anchor tip, where the hoop can deflect so that load can be distributed when the anchor is in contact with an object (e.g. native heart anatomy) over a larger surface area. In the illustrated embodiment, a circular shape (with a diameter R of, e.g., 2-4 mm) is cut into the end of the anchor tip. When compressed by contact with tissue, the circular shape forms an oval shape (as shown in FIG. 2G-24B with a diameter L3 of, e.g., 3-7 mm). The thin-walled hoop can also deflect around or between chordae when in contact. In particular, the anchor tip of FIG. 2G-24B is more tolerable to greater contact loading due to the greater contact surface area to distribute. This anchor tip design may not require any shape setting or welding.

FIGS. 2G-25A and 2G-25B illustrate zig-zag spring anchors each having a zig-zag pattern cut into the tip of the anchor 37 to provide load distribution and cushioning. The zig-zag spring anchor of FIG. 2G-25A may be a slanted zig-zag pattern creating an angle greater than 0° but less than 900 with a length or width L4 (e.g., 2-3 mm) and a height H3 (e.g., 3-5 mm), while the zig-zag spring anchor of FIG. 2G-25B may by curved or bent (e.g., at a right angle of 90° or approximately 90°). This anchor tip design may not require any shape setting or welding.

FIGS. 2G-26A, 2G-26B and 2G-26C illustrate a whisk tip anchor formed by looping multiple wires 37C-4 over and passing through holes 37J around the periphery of a small circular plate 37H to form two to four or more than four wire hoops, where a center rectangular hole 371 of the plate 37H can fit over and be sutured onto the end of an anchor arm. FIG. 2G-26A shows a side view of the whisk tip anchor and FIG. 2G-26B shows a top view of the circular plate 37H and a close-up, side view of an anchor arm that includes a tip configured to receive the hole 371 of the plate 37H. The ends of the wires 37C-4 may be laser welded to the circular plate 37H. The wires 37C-4 that are looped around may optionally be covered be a sleeve or cloth sock 37C. FIG. 2G-26C illustrates a top view of the whisk tip anchor, looking from the top of the wires 37C-4. The wires may comprise nitinol or other shape memory material. For nitinol wires, a different Af temperature may be used for the nitinol wires than for the inner frame 32 (e.g., an Af temperature closer to body temperature), which may facilitate the nitinol wires providing a softer anchor cushion.

FIG. 2G-27A illustrates a cylindrical braid tip anchor formed by a fine wire 37N braided into a cylinder and looped over the anchor 37 to provide cushioning during anchor loading. The fine wire could be nitinol wire, cobalt chrome wire, stainless steel wire, polymer wire, or radiopaque metal wire, and the wire may be tube-shaped. In addition, an optional sleeve or cloth sock 37C may be looped around the fine wire 37N. FIG. 2G-27B shows another embodiment of a cylindrical braid tip anchor having a cone shape formed by an inverted cylinder braid tip. In both cylindrical braid tip anchors in FIGS. 2G-27A and 2G-27B, the end of the inner frame 32 or the anchor 37 may be split into two pinching arms 37P for securing the wire ends with an optional crimp sleeve 370. For nitinol wires, a different Af temperature may be used for the nitinol wires than for the inner frame 32 (e.g., an Af temperature closer to body temperature), which may facilitate the nitinol wires providing a softer anchor cushion.

### Co-Organizing Dual Frame Features

In accordance with several embodiments, it is desirable to provide complementary features on the structural components (e.g., inner and outer frames) of dual-frame transcatheter devices (e.g., prosthetic implants or replacement heart valves). These complementary features may be intended to ensure co-organization of the inner and outer frames. The co-organizing or complementary features and may or may not be in contact in the expanded and/or crimped states. However, these co-organizing features may advantageously interact to help promote alignment of the inner and outer frames during loading and deployment steps, and during any subsequent recapture steps.

The co-organizing or complementary features may be beneficial for device performance, ensuring organized frames for low loading/recapture force, symmetric device profiles during deployment for procedural consistency, and reduced strain concentrations in the frames that commonly result from asymmetric loading and that reduce device durability. Without the use of such co-organizing or complementary features, the structural components (e.g., inner and outer frames) can work against each other (e.g., through fighting for space) and can result in an undesirable asymmetric arrangement that can lead to a more difficult procedure or degradation of the device (e.g., prosthetic implant).

Transcatheter implants (e.g., replacement heart valves) are typically designed for two states, or configurations: an expanded state (e.g., following implantation at a desired location) and a crimped state (e.g., within a delivery device upon manufacture or upon recapturing). Between these two states, the implant undergoes some level or form of transition, such as diametric reduction (e.g., during loading) or expansion (e.g., during implant deployment). This transitory state between the expanded state and the crimped state, often an afterthought in design, can be important, as it can affect the ease and/or safety of the implantation procedure. In some instances, multi-frame (e.g., dual-frame) implants may have undesirable frame-to-frame interaction that creates instability within the implant and can lead to the implant presenting in an undesired, asymmetrical fashion to the anatomy during deployment, which can complicate achieving a successful implantation procedure. Another consequence of negative interaction between the frames can damage the implant cloth or skirt fabric material (e.g., resulting in leaks) and/or can damage the frames (which can lead to reduced frame durability and fatigue or failure).

Various co-organizing or complementary frame features may be designed to ensure that the inner and outer frames, or portions of the frames, remain aligned and organized throughout the transitory states. FIG. 2H shows a side view of an embodiment of an outer frame 34 including co-organizing frame features. The co-organizing frame features include features of a proximal portion 34A of the outer frame 34 and a distal portion 34C of the outer frame 34. The specific co-organizing frame features will be discussed in more detail below. In some implementations, the co-organizing or complementary frame features are designed to only engage one another during the transitory states. The co-organizing or complementary frame features may be designed to, for example, reduce degrees of freedom, link up to protect delicate sections or portions of the implant, or work like a seal to progressively join the frames in an organized fashion.

As one example, an outer frame of a dual-frame implant may include a structural component configured to engage with a portion of the inner frame of the dual-frame implant upon expansion and/or compression of the dual-frame implant (e.g., during a transition state) so as to reduce a likelihood of rotational and/or translational movement between the outer frame and the inner frame. FIGS. 2I-1 to 2I-3 illustrate various proximal eyelet designs configured to reduce rotational and/or translational movement between an outer frame and an inner frame of a dual-frame implant. FIG. 2I-1 shows a close-up view of proximal portions 32A, 34A of embodiments of the inner frame 32 and the outer frame 34 during a transitory state in which the proximal portions 32A, 34A are in a crimped configuration but the distal portions 32C, 34C are still expanded. As shown, the proximal eyelets of the outer frame 34 can include a hammer-head design to provide uniform spacing between the eyelets. The hammer-head design includes thickened side walls with flat edge surfaces for the upper eyelet and the lower eyelet of the outer frame 34. The thickened flat side surfaces of the eyelets are configured to contact and abut against each other to provide the uniform spacing (due to uniform dimensions of the design). FIG. 2I-2 shows a portion of a flat cut pattern of an embodiment of the outer frame 34 that shows one eyelet portion having a hammer-head design adapted to restrict rotational freedom of movement only. FIG. 2I-3 shows a portion of a flat cut pattern of an embodiment of the outer frame 34 that shows two adjacent eyelet portions having a hammer-head design configured to restrict rotational and/translational freedom of movement. As shown, the eyelet portions (shown at the top of FIGS. 2I-2 and 2I-3) include two central extensions (e.g., nubs, protrusions, tabs) on one side of a central eyelet configured to engage with a cut-out feature (e.g., recess, notch, indentation) on an opposite side of an adjacent central eyelet to restrict translational height movement when adjacent eyelet portions are engaged. The upper and lower eyelets include thickened side portions that are wider/thicker on one side than on the other side. Other designs and shapes may be used to facilitate co-organization between eyelet portions of the outer frame 34.

FIGS. 2J-1 and 2J-2 help to illustrate another example of a co-organizing frame feature (e.g., slot, opening, or guide structure) of an outer frame designed to straddle an inner frame axial strut (e.g., the inner frame axial strut extends outward within the co-organizing frame feature of the outer frame) to facilitate alignment of the outer frame and the inner frame during transition between an expanded configuration and a compressed configuration, and/or vice-versa. The outer frame may have multiple co-organizing frame features spaced circumferentially around the outer frame to straddle multiple inner frame axial struts. FIG. 2J-1 illustrates a dual-frame design without co-organizing frame features. As shown, overlaid axial beams of an outer frame with a high degree of curvature in an expanded state results in non-uniform geometry in a transitory state. FIG. 2J-2 illustrates a dual-frame design with co-organizing frame features. The outer frame 34 includes the hammer-head proximal eyelet design described previously and shown in FIG. 2I-1. The complementary or co-organizing frame feature of the inner frame 32 is an axial beam or strut 212 on a proximal, or inflow aspect, 32A of the inner frame 32. The complementary or co-organizing frame feature of the outer frame 34 may be a wide diamond cell junction at the proximal, or inflow, aspect 34A of the outer frame 34, which overlaps a tightly-radiused segment of the shape profile. In some embodiments, the co-organizing frame feature of the outer frame 34 comprises a C-shaped or U-shaped junction (e.g., forming a slot, or guide receptacle or other mechanism) designed to straddle the corresponding inner frame axial strut 212 for alignment. As the dual-frame implant is loaded into a delivery device, the radiused outer frame C-shaped junction bends inward and straddles the inner frame axial strut 212, which acts as a vertical rail and helps keep the outer frame 34 perfectly or nearly perfectly aligned to the inner frame 32 throughout loading, recapturing, repositioning. Once the implant is fully crimped, the curvature of the outer frame co-organizing frame feature (e.g., C-shaped or U-shaped junction) is straightened out and disengages from the inner frame axial strut 212. The co-organizing frame features may not be engaged (or may not interact) when the implant is in a fully-expanded configuration.

FIG. 2K-1 illustrates how an outer frame without co-organizing frame features can adversely interact with an anchor 37 on an inner frame of a dual-frame valve prosthesis during crimping. FIG. 2K-2 shows how an embodiment of an outer frame 34 can be designed to include a co-organizing frame feature such that distal outflow portion 34C of the outer frame 34 avoids interaction with inner frame anchors 37 during crimping. The distal outflow portion 34C of the outer frame 34 may be shaped and adapted such that the distal apexes of the distal cells of the outer frame 34 do not align with or overlap with the distal anchors (e.g., ventricular anchors) 37 of the inner frame 32. The anchors 37 may instead be designed to be located between the distal apexes of the distal cells of the outer frame 34 during crimping.

### Proximal/Inflow/Inlet Strut Features

FIG. 2J-3 illustrates an embodiment of an inner frame 32 design in which the proximal, or inlet, struts are at uneven, staggered, or offset heights in order to reduce a total (i.e., maximum) force required to retrieve and recapture a fully-expanded or partially atrially-expanded replacement heart valve, or valve prosthesis. The offset, staggered, or uneven heights distributes the force during recapture, rather than having one large spike at once as all the struts are pulled into a delivery system simultaneously, as is the case when the heights are all uniform and not offset (e.g., are axisymmetric). FIG. 2J-4 is a graph showing expected results of force reduction using the offset height design of FIG. 2J-3. Referring to FIG. 2J-3, proximal, or inlet, struts 202 of an inner frame 32 may have different heights (e.g., height difference H4) in a way that adjacent struts are offset relative to one another. The alternating, offset heights allow half of the struts 202 to be pulled into the delivery system first, and the remainder to be pulled in subsequently, thus creating two small spikes in recapture force rather than one large spike as shown in FIG. 2J-4. The force reduction may be, for example, a 25-50% reduction in force. That is, the offset configuration can create sequential seating of the struts 202 inside a pusher 506 or capsule tip of a capsule subassembly 306, lower recapture forces, reduce tension on the recapture sutures, reduce force on the dual-frame valve, and reduce compression during the recapturing process. Accordingly, a reduction in force to load a valve prosthesis and recapture a valve prosthesis is expected. The recapture force reduction may result in less tension on the suture during recapture and less compression on the mid-shaft subassembly 22 during recapture. The staggered or offset heights may also help reduce risk of a strut catching on a dstial tip or edge of the capsule subassembly 306 as the implant 30 is recaptured within the capsule subassembly 306. The heights of the struts 202 can be varied by, for example, changing the strut length (e.g., height above a connection point to a main frame body (e.g., cell structure)), angle, or the like. There may be two different heights, with the height alternating with each strut around a circumference of the frame. There may be more than two different heights (e.g., three different heights, four different heights), with different pairs or groups of struts having different heights.

In accordance with several configurations, an outer frame of a dual-frame implant may include a cantilevered or hinged attachment tab that allows attachment between the outer frame and an inner frame in a manner that allows an angle to be formed between the attached portions of the outer frame and the inner frame because the attached portion of the outer frame bends on an independent plane from the attached portion of the inner frame, thereby reducing a radius of curvature of the dual-frame implant along the region where the outer frame and the inner frame are attached. FIGS. 2L-1 to 2L-3 illustrate various examples of an attachment or connection structure between proximal eyelets and connecting struts of an outer frame of a dual-frame valve prosthesis. FIG. 2L-1 shows that a bottom (e.g., distal-most or lower-most) eyelet of the eyelets 35 of the proximal tab 33 of the proximal portion 34A of the outer frame 34 may be connected to the proximal end of one or more struts 34E, 34F of the outer frame 34 by a bridge 34G. The struts can include at least two outer strut legs 34E connected to the bridge 34G. The struts may further include at least two inner leg struts 34F of which one end is coupled to an upper inner portion of a respective one of the at least two outer leg struts 34E. The bridge 34G may have a predetermined length between the lowermost eyelet of the eyelets 35 and a junction C. In addition, the at least two outer legs 34E may extend downwards from the junction C. When at least one of the plurality of eyelets of each of the plurality of tabs of the outer frame 34 is engaged with at least one of the plurality of eyelets of the plurality of tabs of the inner frame, the bridge 34G of each connection structure is in surface contact with a respective tab of the inner frame 32. In several instances, this design may require tangency with the inner frame eyelets when the outer frame 34 and the inner frame 32 are aligned and engaged together, which can force a high radius of curvature profile that can result in high strain during crimping and a concentrated fatigue strain on the reverse taper of the junction C between the bridge 34G and the proximal end of the struts.

FIG. 2L-2 shows another example of a linking element or connection structure of the outer frame 34. In this example, the bridge 34G has been substantially shortened compared to that shown in FIG. 2L-1. The bridge 34G is not connected to the bottom, or distal-most, eyelet but is connected to a proximal-most, or upper, eyelet via an outer framework 341 of the tab 33 that extends from the bridge 34G and surrounds the more distal eyelets, thereby forming a “pop tab” configuration like the tab used to open a can of soda pop. The bridge 34G in FIG. 2L-2 may have the same or shorter length than that in FIG. 2L-1. Similar to the embodiment of FIG. 2L-1, the at least two outer leg struts 34E in FIG. 2L-2 may extend downwards from the junction C. The bridge 34G of FIG. 2L-2 may advantageously separate planes of movement such that the tab 33 can bend along a plane independent of the outer framework 341, the bridge 34G, and/or the outer leg struts 34E and independent of the attached portion of the inner frame 32. Thus, the attached portion of the outer frame 34 can bend at an angle with respect to the attached portion of the inner frame 32, thereby facilitating a reduced radius of curvature along the proximal inflow regions of the dual-frame implant or valve prosthesis.

FIG. 2L-3 shows still another example of a linking element or connection structure of the outer frame 34. As shown in FIG. 2L-3, the bridge 34G and junction C have been removed completely from the structure. The at least two outer leg struts 34E are connected to respective sides of the upper-most, or proximal-most eyelet but do not connect to respective sides of the other eyelets, thus forming a “paper clip” configuration. extend downwards from the outer tab 33B, and the inner tab 33A can be spaced apart from the at least two outer legs 34E along at least a portion of an edge of the inner tab 33A.

In accordance with several embodiments, the geometry implementations of FIGS. 2L-2 and 2L-3 advantageously eliminate the requirement for eyelet tangency with the connecting struts by creating an independent plane (e.g., bendable or cantilever tab portion) for eyelet attachment between one or more attachment eyelets of the inner frame 32 and outer frame 34 and provides more flexibility for future profile designs of the outer and inner frames. For example, the inflow struts on the bendable or cantilever tab portion of the outer frame may act as a cantilever that keeps the outer frame 34 closed until the capsule subassembly 306 is fully retracted.

FIGS. 2L-4 to 2L-6 illustrate various embodiments of tabs 33 and/or eyelets 35 of proximal, or inlet, struts of an outer frame. In accordance with several implementations, these embodiments may advantageously prevent, or reduce the likelihood of, suture or tether loops being cow hitched, looped, or “locked” around a tip of a proximal, or inlet, strut during removal of the suture or tether during a step of releasing the valve prosthesis from attachment to the delivery system. Instead, the suture or tether loop can be readily disconnected from the outer frame 34 of the valve prosthesis through the uppermost or proximal-most eyelet 35A. In particular, FIGS. 2L-4 and 2L-5 show a linking element or connection structure of the outer frame 34, similar to FIG. 2L-2 forming a “pop tab” configuration like the tab used to open a can of soda pop. However, the embodiments of FIGS. 2L-4 to 2L-6 can be incorporated into the “paper clip” configuration or other configurations as well. The embodiments of FIGS. 2L-4 and 2L-5 can be formed by laser cutting. An uppermost or proximal-most eyelet 35A may have a half circle (semi-circle) shape (such as shown in FIG. 2L-4), an oval shape (such as shown in FIG. 2L-5), or a bean shape (such as shown in FIG. 2L-6). The uppermost or proximal-most eyelet 35A may have a generally rounded geometry as shown in these figures. Further, a height H5 of an attachment hole centerline of the proximal-most eyelet 35A may be varied (e.g., decreased) such that the suture or tether cannot catch, loop or hitch on the proximal tip of the proximal, or inlet, strut. With reference to FIG. 2L-6, the proximal-most eyelet 35A has a radius R that is greater than that of FIG. 2L-4 but smaller than that of FIG. 2L-5. In one embodiment, the radius R may be about 0.1 mm to 0.3 mm. The height H6 and the height H7 combine to be the height H5. Either or both height H6 and height H7 can be reduced to reduce height H5. The height H6 may range from 0.230 mm to 0.330 mm in some embodiments and the height H7 may range from 0.520 to 0.580 mm in some embodiments. By reducing either or both height H6 and height H7, and thus reducing height H5, the thickness of the suture or tether, in combination with the reduced height, prevents the suture or tether from looping, catching, or hitching, on the proximal tip of the proximal, or inlet, strut. The proximal tip of the proximal, or inlet, strut may also have a rounded or chamfered outside top geometry. For example, the proximal tip of the proximal, or inlet, strut may have a radius of curvature R2. In accordance with several embodiments, the radius of curvature R2 is designed to be less than the height H5. The side geometry of the proximal, or inlet, strut may be straight in some embodiments (such as shown in FIGS. 2L-2 to 2L-5) as opposed to a “snowman” side geometry (such as shown in FIG. 2L-1).

Different tab configurations particularly varying the eyelet configuration as described above can bring different advantages such as ease of manufacturing the outer frame, ease of attachment of the replacement heart valve (e.g., by suturing), reduction of tensile stress, etc. In accordance with several embodiments, a series of maneuvers (e.g., posterior, anterior, lateral, and medial maneuvers) may be performed during the tether/suture release step to provide an indication of any likelihood of hitching or looping.

FIGS. 2M-1 and 2M-2 illustrate various radii of curvature profiles of a dual-frame valve when an inner frame and an outer frame are engaged. For instance, when the embodiment of the outer frame of FIG. 2L-1 is engaged with the inner frame, the outer profile may have a radius of curvature as shown in FIG. 2M-1, while when the embodiments of FIG. 2L-2 or FIG. 2L-3 are engaged with the inner frame, the outer profile may have a radius of curvature smaller than that in FIG. 2M-1, as shown in FIG. 2M-2. A high radius of curvature may make it challenging for a physician to capture chordae tendineae beneath the annulus of a mitral valve, for example, because the outer frame of the dual-frame valve prosthesis may have to be deployed at the same time the ventricular anchors reach their full diameter. Thus, by changing the configuration of the outer frame, more specifically, by changing the configurations of the eyelet and strut connection or attachment structures of the outer frame, the radius of curvature of the dual-frame heart valve prosthesis can be adjusted so as to delay the deployment of the outer frame in addition to reducing crimping strains at locations that undergo compound radial and circumferential bending due to the curvature of the profile, e.g., at the junction C. Thus, the dual-frame valve prosthesis may be designed to have a reduced radius of curvature at a proximal end when in the expanded configuration, as shown in FIG. 2M-2.

In accordance with several embodiments, the implementations shown in FIGS. 2L-2 and 2L-3 and 2M-2 provide flexibility to create a new cork profile for the dual-frame valve prosthesis. The connection structures shown in FIGS. 2L-2 and 2L-3 and the more gradual profile or radius of curvature of FIG. 2M-2 may allow for a delayed release of the outer frame during delivery and may reduce both crimp strain and fatigue strain. The delayed release may be accomplished by using the inflow struts as a cantilever that keeps the outer frame closed until a delivery capsule (e.g., capsule subassembly 306 described below) is fully retracted. The reduced radius of curvature may provide a significant reduction in fatigue strain at junction C and an improved crimp strain distribution.

FIGS. 2N-1 and 2N-2 illustrate an outer frame having the “pop tab” connection structure design of FIG. 2L-2 in an expanded configuration and shows the reduced radius of curvature profile of this design. FIGS. 2N-3 and 2N-4 illustrate an outer frame having the “paperclip” connection structure design of FIG. 2L-3 in an expanded configuration and shows the reduced radius of curvature profile of this design.

FIG. 20-1 illustrates a dual-frame valve prosthesis in which an inner frame 32 and an outer frame 34 are engaged in a pre-expansion state where the outer frame 34 is not deployed. FIG. 20-2 illustrates a dual-frame valve prosthesis in which an inner frame 32 and an outer frame 34 are engaged in a capsule retracted state where the outer frame 34 is deployed. As described herein, by changing the linking or connection structure (e.g., shapes, connections, etc.) of a proximal portion of the outer frame 34, it is possible to delay the deployment of the outer frame 34 as shown in FIG. 20-2.

In some examples, the outer frame and the inner frame of the dual-frame valve prosthesis may be engaged by aligning and attaching one or more of the plurality of eyelets 35 thereof, for example, in a “snowman” method of inner and outer frame fixation. The larger diameter of the outer frame can be served to engage with the native anatomy for the purposes of sealing and securement in the large annulus native anatomy. The smaller inner diameter of the inner frame can serve to hold tissue leaflets of a prosthetic valve and can provide a smaller prosthetic valve diameter to reduce tissue bulk, pulsatile frame loading, and frame radial crimping forces. The dual-frame valve prosthesis structures can provide the above advantages by creating an appreciable difference between the expanded diameters of the inner and outer frames.

In certain embodiments, proximal eyelet portions of the inner frame and the outer frame may be engaged with each other adapting the “snowman” method of aligning eyelets of each frame and wrapped sutures multiple times through the aligned inner and outer eyelets to hold the frame struts together at the inflow side of the valve. To maintain the appreciable difference in expanded diameters between the inner and outer frames assembled using the “snowman” methods described above, sharp bends are needed to create space between the inner and outer frames, resulting in increased strains and crimp loading forces. For instance, referring to FIGS. 2P-1 and 2P-2 which show eyelets 35 of each of the inner frame 32 and the outer frame 34 being engaged with each other by a suture looping around each of the eyelets a predetermined number of times to secure the attachment of the eyelets. Here, the outer frame 34 may have an attachment configuration corresponding to the example of FIG. 2L-1 described above.

FIGS. 2P-3 and 2P-4 illustrate another example of connecting or engaging the inner frame 32 and the outer frame 34 of a dual-frame valve prosthesis. For example the inner frame 32 and the outer frame 34 may include corresponding, or complementary, engagement or attachment features that allow for an angle to be formed between the engaged portions of the inner frame 32 and the outer frame 34 at the attachment point. In the examples of FIGS. 2P-3 and 2P-4, an inner lock tab member 33A of the tab 33 of the outer frame 34 comprises a puzzle piece lock tab end configured to fit within a corresponding slot on a proximal inflow end of a corresponding tab or strut 202 of the inner frame 32, thereby providing a compact mechanical lock between the strut of the inner frame 32 and the inner lock tab member 33A of the outer frame 34. As shown in FIG. 2P-3, the “puzzle piece lock tab” design may advantageously enable a larger angle between the inner frame and the outer frame at the attachment point than the embodiment of FIGS. 2P-1 and 2P-2 and may provide a more gradual curve profile for the outer frame 34, thereby reducing strain and crimp loading force.

For certain embodiments, the inner lock tab member 33A comprises a joint (e.g., dovetail-shaped joint) that fits within a correspondingly-shaped slot of the strut 202 of the inner frame 32 (e.g., a simple planar fit), thus reducing a load off the suture by the mechanical lock between the interacting metal components of the frames. The connection or engagement can involve use of a single suture lashing to keep the two frames coplanar at the joint point or mechanical fit interface, or can optionally involve off-center/off-axis laser cutting that can provide a tapered or beveled fit between the tabs 33 of the outer frame 34 and the inner frame 32 to reduce the suture usage while keeping the tab 33 of the outer frame 34 and the inner frame 32 coplanar by the spring force of the tab holding the frames together as shown in FIG. 2P-4. FIG. 2P-4 also shows a detailed cross-section view along section line B-B. The detailed cross-section view shows in more detail how the interface between the inner lock tab 33A and the strut tab opening of the inner frame 32 can optionally be beveled with off-axis laser cutting to lock the metal tabs of the inner frame and the outer frame together without requiring any sutures.

In another example, referring to FIGS. 2P-5 and 2P-6, the proximal ends of the inner frame and outer frame may be connected or joined using a dovetail joint connection structure. This embodiment can provide a dovetail joint in which a proximal end or strut of the inner frame 32 comprises a dovetail shape (e.g., cut with a perpendicular laser cutting operation), while a strut of the outer frame 34 has an angled cut to match the angle of the dovetail joint member on the inner frame 32, thereby forming a dovetail joint or matched fit that allows fitting the parts together one way but preventing the parts from pulling apart any other way. The dovetail angle of the inner frame 32 and the off-center taper angle of the strut of the outer frame 34 can be adjusted to allow for different angle between inner frame 32 and the outer frame 34 (e.g., 45 degrees, 60 degrees, 90 degrees, or other angle). Two alternative optional techniques for preventing the inner and outer frames from coming apart (e.g., inner frame dovetail member backing out of dovetail groove on outer frame under loading conditions) include (1) that eyelets 35 of the inner frame and the outer frame may be optionally engaged to each other by a tensioned suture or tether wrapping therethrough and/or (2) the outer frame may comprise a snap lock 34J integrally or detachably connected to a strut of the outer frame to secure the attachment of the inner frame 32 and the outer frame 34, as shown in FIG. 2P-6.

The joint structure as illustrated in FIGS. 2P-3 and 2P-4 or FIGS. 2P-5 and 2P-6 can advantageously facilitate achievement of a greater angle between the inner frame and outer frame at the attachment point, while also reducing valve space in the crimp length direction and avoiding total reliance on suture wraps for fixation. The joint structure as illustrated in FIGS. 2P-3 and 2P-4 or FIGS. 2P-5 and 2P-6 can also advantageously provide for easier access and sewing during manufacture of the connection structure. FIG. 2P-7 illustrates a close-up view of another example of a dovetail joint structure. As shown, one or more dovetail tabs can be formed to provide secure engagement.

### Delivery Device

Referring briefly back to FIG. 1, the delivery device 15 can include a shaft assembly 12 comprising a proximal end and a distal end, with a handle 14 coupled to the proximal end of the shaft assembly 12. The delivery device 15 can be used to hold the implant (e.g., prosthesis, replacement heart valve) for advancement of the same through the vasculature to a treatment location. In some embodiments, the shaft assembly 12 can hold at least a portion of an expandable implant (e.g., prosthesis, replacement heart valve) in a compressed state for advancement of the implant within the body. The shaft assembly 12 may then be used to allow controlled expansion of the implant at a desired implantation location (e.g., treatment location). In some embodiments, the shaft assembly 12 may be used to allow for sequential controlled expansion of the implant as discussed in detail below.

The shaft assembly 12 of the delivery device 15 can include one or more subassemblies, such as an outer sheath subassembly 20, a rail subassembly 21, a mid-shaft subassembly 22, a release subassembly 23, a manifold subassembly 24, and/or a nose cone subassembly, as will be described in more detail below. In some embodiments, the shaft assembly 12 of the delivery device 15 may not have all of the subassemblies disclosed herein. The delivery device 15 may include multiple layers of concentric subassemblies, shafts, or lumens. The various lumen or shaft subassemblies will be described starting from an outermost layer. In some embodiments, the subassemblies disclosed below may be in a different radial order than is discussed.

### Outer Subassembly

FIG. 3A shows a perspective view of an embodiment of the outer sheath subassembly 20 of the delivery device 15 of the delivery system 10. The outer sheath subassembly 20 forms a radially outer covering, or sheath, to surround an implant retention area and prevent at least a portion of the implant (e.g., replacement heart valve or valve prosthesis) 30 from radially expanding until ready for implantation. Specifically, the outer sheath subassembly 20 can prevent a distal end portion of the implant 30 from radially expanding.

The outer sheath subassembly 20 can include an outer proximal shaft 302 having a proximal end portion operably coupled (e.g., via threaded outer sheath adapter 303) to a capsule knob 905 (which may be a distal-most knob, as shown in FIGS. 9A and 9B) of the handle 14 such that rotation of the capsule knob 905 causes proximal and distal translation of the outer sheath subassembly 20 (e.g., clockwise and counter-clockwise rotation). A capsule subassembly 306 can be attached to a distal end of the outer proximal shaft 302. The components of the outer sheath subassembly 20 can form an outer-most lumen for the other subassemblies to pass through.

The outer proximal shaft 302 may be a tube formed of a plastic, but could also be formed of a metal hypotube or other material. The outer proximal shaft 302 may include an outer jacket or liner made of fluorinated ethylene propylene (FEP) material, polytetrafluoroethylene (PTFE) material, ePTFE material, or other polymeric material so as to make the outer surface of the outer proximal shaft 302 smooth and/or hemostatic. The outer proximal shaft 302 may include a connector (e.g., flexible reflow member) at its distal end to facilitate connection or coupling to the capsule subassembly 306. At least a portion of the outer proximal shaft 302 may comprise a laser cut hypotube with a universally flexible pattern (e.g., an interrupted spiral pattern or an interrupted coil).

FIG. 3B shows a side cross-section view of the capsule subassembly 306. The capsule subassembly 306 may include a distal hypotube, or capsule stent, 308, an inner liner inside of the hypotube 308, a distal capsule tip 309, and one or more outer liners or jackets 311 surrounding the hypotube 308. The one or more outer liners or jackets 311 may comprise polyether block amide (e.g., PEBAX® material) or other suitable polymer or thermoplastic elastomer material, such as polytetrafluoroethylene (PTFE) or expanded polytetrafluoroethylene (ePTFE). The inner liner may comprise PTFE, which may be pre-compressed before application to the inside of the hypotube 308. The distal capsule tip 309 may comprise an atraumatic tip adapted to act as a funnel to facilitate recapture (e.g., crimping) of a valve prosthesis or other implant. The distal capsule tip 309 may be comprised of polyetheretherketone (PEEK) or other thermoplastic, polymeric, or metallic material. The distal capsule tip 309 may be loaded with radiopaque material (e.g., 5-40% barium sulfate loading) to facilitate detection (e.g., made fluorogenic) under radiographic imaging (e.g., fluoroscopy). The distal capsule tip 309 may fit within an open distal end of the hypotube 308.

FIG. 3C shows a perspective view of the distal hypotube, or capsule stent 308. The capsule stent 308 can be formed from one or more materials, such as PTFE, ePTFE, polyether block amide (e.g., PEBAX), polyetherimide (e.g., Ultem® material), PEEK, urethane, Nitinol, stainless steel, and/or any other biocompatible material. The capsule stent 308 is preferably flexible while still maintaining a sufficient degree of radial strength to maintain an implant (e.g., replacement valve) 30 within the capsule stent 308 without substantial radial deformation, which could increase friction between the capsule stent 308 and an implant contained therein. The capsule stent 308 also preferably has sufficient column strength to resist buckling, and sufficient tear resistance to reduce or eliminate the possibility of the implant tearing and/or damaging the capsule stent 308. The proximal end and/or distal end of the distal hypotube, or capsule stent 308 may include multiple laser cut windows 313 adapted to make the proximal and/or distal end fluorogenic and/or echogenic to facilitate visualization under certain imaging modalities (e.g., noninvasive ultrasound imaging or invasive fluoroscopic imaging). In several implementations, a separate radiopaque element or member is not added to the hypotube 308 to facilitate imaging because of the presence of the laser cut windows 313. The laser cut windows 313 may also promote adhesion of the outer jacket 311 to the capsule stent 308 and to the inner liner(s) by allowing glue or other adhesive to flow through the laser cut windows 313. One or more layers of connection members made of PEBAX or other suitable material may surround the laser cut windows 313 to facilitate coupling of the hypotube, or capsule stent 308 to the distal capsule tip 309.

The hypotube 308 may be formed of a plastic or metallic material. In some implementations, the hypotube 308 can be a metal hypotube. If metallic, the metallic material of the hypotube 308 may comprise cobalt chrome, stainless steel, titanium or metal alloy, such as nickel-titanium alloy material. The coil construction or cut patterns of the outer proximal shaft 302 and/or the hypotube 308 can allow the outer proximal shaft 302 to follow the rail subassembly 21 in any desired direction. A cut pattern of the outer proximal shaft 302 and/or the hypotube 308 may be modified (e.g., cut per revolution, pitch, spine distance) to control tension resistance, compression resistance, flexibility, and torque resistance. For example, cuts per revolution may range between 1.5 and 5.5, pitch may range between 0.005″ and 0.15″, and spine distance may range between 0.015″ and 0.125″. The hypotube 308 may advantageously provide both tension and compression. The one or more outer liners or jackets 311 may allow the capsule subassembly 306 to be more flexible. The capsule hypotube 308 can bend in multiple directions. In some implementations, a distal terminus of the outer liner or jacket 311 may be positioned proximal of the distal terminus of the hypotube 308.

The capsule subassembly 306 may have a similar diameter as the outer proximal shaft 302 or a different diameter. In some embodiments, the capsule subassembly 306 has a uniform or substantially uniform diameter along its length. In some embodiments, the capsule subassembly 306 can be 28 French or less in size (e.g., 27 French). In some embodiments, the capsule subassembly 306 may include a larger diameter distal portion and a smaller diameter proximal portion. The capsule subassembly 306 can be configured to retain the implant (e.g., valve prosthesis) 30 in the compressed position within the capsule subassembly 306 (e.g., within an implant retention area 316 occupying a distal-most ˜2 inches (or ˜50 mm) of the capsule subassembly 306.) Additional structural and operational details of a capsule subassembly, such as those described in connection with capsules in U.S. Publication No. 2019/0008640 and U.S. Publication No. 2019/0008639, which are hereby incorporated by reference herein, may be incorporated into the capsule subassembly 306.

The outer sheath subassembly 20 is configured to be individually slidable (translatable) with respect to the other assemblies by rotation of the capsule knob 905. Further, the outer sheath subassembly 20 can slide (translate) distally and proximally relative to the rail subassembly 21 together with the mid-shaft subassembly 22, manifold subassembly 24, release subassembly 23, and/or nose cone subassembly.

FIG. 3D schematically illustrates how at least a portion of a length of one or more components of the capsule subassembly 306 (e.g., inner liner 310) can include excess material such that the capsule subassembly 306 includes built-in slack along a portion of its length (e.g., a portion of the length proximal to the implant retention area 316) to facilitate flexible bending of the capsule subassembly 306 (e.g., to navigate tight turns within a heart or vasculature surrounding the heart).

FIGS. 3E to 3G illustrate an alternative embodiment of a distal capsule tip of capsule subassembly 306. Comparing with the distal capsule tip 309 of FIG. 3B (which has a straight shape end or a perpendicular flush cut distal end), distal capsule tip 309A has an uneven end (e.g., lobed tip or waved shape), as shown in FIG. 3E, formed by alternately protruding and recessed lobes 309A-1 and 309A-2. Such an uneven end of the distal capsule tip 309A allows a staged deployment or recapture of anchors 37. FIG. 3F illustrates a side partial cross-section view of the distal capsule tip 309A and schematically shows the staged or offset deployment or recapture of anchors 37 due to the lobed design of the distal capsule tip 309A. FIG. 3G is a flat plan view that illustrates that when recapturing anchors, the capsule subassembly 306 can recapture, for example, one or more (e.g., two, three or more) anchors 37 first, then the remaining anchors 37 (either individually or in pairs, trios, or other groupings) at subsequent stages (e.g., two or three stages). The staged recapture or deployment can advantageously distribute the recapture force to straighten the anchors 37 over time to reduce the overall force amplitude (e.g., by 20-40%, for example) at any one time during recapture. In this example, three lobes are shown, at 12 o'clock, 4 o'clock, and 8 o'clock. Such a configuration allows some anchors to begin unflexing before others during the recapture process in which the capsule is advanced over J-shaped anchors. This staggering or staging of the anchor recapture distributes the forces to unflex the anchors and advance the capsule, thus reducing peak loads or forces. Other numbers of lobes or shapes of lobes may be used.

### Rail Subassembly

FIG. 4A shows a perspective view of a rail subassembly 21 of the delivery device 15 of the delivery system 10 of FIG. 1. FIG. 4A shows approximately the same view as FIG. 3A, but with the outer sheath subassembly 20 removed, thereby exposing the rail subassembly 21. FIG. 4B further shows a cross-section of the proximal and distal end portions of the rail subassembly 21 to view the pull wires that facilitate steering of the rail subassembly 21. The rail subassembly 21 can include a rail shaft 402 (or rail) generally attached (and operably coupled) at its proximal end to the handle 14. The rail shaft 402 can be made up of a rail proximal shaft 404 directly attached to the handle 14 at a proximal end and a rail hypotube 406 attached to the distal end of the rail proximal shaft 404 (e.g., via a connector, ring-like structure, or insert 407). The rail subassembly 21 is operably coupled to the handle 14 via primary flex adapter 403A (which controls medial-lateral trajectory of the distal end portion of the rail subassembly 21 via one or more distal pull wires 410A), via secondary flex adapter 403B (which controls anterior-posterior trajectory of the distal end portion of the rail subassembly 21 via one or more proximal pull wires 410B), and via rail adapter 405 (which includes a side needleless injection port to facilitate flushing and de-airing functions). The rail proximal shaft 404 may include an interrupted spiral cut pattern along a large portion of its length to facilitate compression. The rail hypotube 406 can further include an atraumatic rail tip 408 at its distal tip. The atraumatic rail tip 408 may not comprise slits and is configured to extend up to 1 inch beyond the distal terminus of the rail hypotube 406 and is configured not to dig into the outer shaft subassembly 20 to avoid friction and fatigue and to prolong use. These components of the rail subassembly 21 can form a rail lumen for the other inner subassemblies to pass through.

FIG. 4B shows a side cross-section view of the rail subassembly 21 of FIG. 4A. As shown in FIG. 4B, attached to an inner surface of the rail hypotube 406 are one or more pull wires 410 which can be used apply forces to the rail hypotube 406 and steer the rail subassembly 21. The pull wires 410 can extend distally from the primary and secondary flex knobs 915 (illustrated in FIGS. 9A and 9B) in the handle 14 to the rail hypotube 406. In some embodiments, pull wires 410 can be attached at different longitudinal locations on the rail hypotube 406, thus providing for multiple bending locations in the rail hypotube 406, allowing for multidimensional steering. For example, the rail hypotube 406 may provide a primary bend or flex along a medial/lateral trajectory and a secondary bend or flex along an anterior/posterior trajectory.

The rail hypotube 406 can include a number of circumferential slots (e.g., laser cut into the hypotube) to facilitate bending and flexibility. The rail hypotube 406 can generally be broken into a number of different sections. At the most proximal end is an uncut (or unslotted) hypotube section corresponding to the location of insert 407. Moving distally, the next section is the proximal slotted hypotube section 406P. This section includes a number of circumferential slots cut into the rail hypotube 406. Generally, two slots are cut around each circumferential location forming almost half of the circumference. Accordingly, two backbones are formed between the slots extending up the length of the rail hypotube 406. This is the section that can be guided by the proximal pull wire(s) 410B. Moving further distally is the location where the proximal pull wires 410 connect, and thus slots can be avoided. This section is just distal of the proximally slotted section 406P and may correspond to the location of insert, or pull wire connector, 411.

Distally following the proximal pull wire connection area is the distal slotted hypotube section 406D. This section is similar to the proximal slotted hypotube section 406P, but may have significantly more slots cut out in an equivalent length. Thus, the distal slotted hypotube section 406D may provide easier bending and an increased bend angle compared to the proximal slotted hypotube section 406P. In some embodiments, the proximal slotted section 406P can be configured to experience a bend of approximately 90 degrees with a bend radius of between 0.25″ and 1″ (e.g., between 0.25″ and 0.75″, between 0.4″ and 0.6″, between 0.5″ and 1″, overlapping ranges thereof, or any value within the recited ranges), whereas the distal slotted section 406D can bend at approximately 180 degrees with a bend radius of between 0.25″ and 1″ (e.g., between 0.25″ and 0.75″, between 0.4″ and 0.6″, between 0.5″ and 1″, overlapping ranges thereof, or any value within the recited ranges). Further, as shown in FIGS. 4A and 4B, the spines of the distally slotted hypotube section 406D are circumferentially offset from the spines of the proximally slotted hypotube section 406P. Accordingly, the two sections will achieve different bend patterns, allowing for three-dimensional steering of the rail subassembly 21. In some embodiments, the spines can be offset 30, 45, or 90 degrees, though the particular offset is not limiting. At the distal-most end of the distal slotted hypotube section 406D is the distal pull wire connection area which is again a non-slotted section of the rail hypotube 406.

In some embodiments, one distal pull wire 410A can extend to a distal section (e.g., to rail tip 408) of the rail hypotube 406 and two proximal pull wires 410B can extend to a proximal section of the rail hypotube 406; however, other numbers of pull wires can be used, and the particular amount of pull wires is not limiting. For example, two distal pull wires 410A can extend to a distal location and a single proximal pull wire 410B can extend to a proximal location. In some embodiments, ring-like structures or inserts attached inside the rail hypotube 406, known as pull wire connectors, can be used as attachment locations for the proximal pull wires 410B, such as insert 411. In some embodiments, the pull wires 410 can directly connect to an inner surface of the rail hypotube 406.

The distal pull wire(s) 410A can be connected (either on its own or through rail tip connector 408) generally at the distal end of the rail hypotube 406. The proximal pull wire(s) 410B can connect (either on their own or through the insert 411) at a location approximately one quarter, one third, or one half of the length up the rail hypotube 406 from the proximal end. In some embodiments, the distal pull wire(s) 410A can pass through a small diameter pull wire lumen (e.g., tube, hypotube, cylinder) attached on the inside of the rail hypotube 406. This can prevent the pull wires 410 from pulling on the rail hypotube 406 at a location proximal to the distal connection. Further, the lumen can comprise compression coils to strengthen the proximal portion of the rail hypotube 406 and prevent unwanted bending. Thus, in some embodiments the lumen is only located on a proximal portion (e.g., proximal half) of the rail hypotube 406. In some embodiments, multiple lumens, such as spaced longitudinally apart or adjacent, can be used per distal pull wire 410A. In some embodiments, a single lumen is used per distal wire 410A. In some embodiments, the lumen can extend into the distal portion (e.g., distal half) of the rail hypotube 406. In some embodiments, the lumen is attached on an outer surface of the rail hypotube 406. In some embodiments, the lumen is not used. In some embodiments, one or more compression coils 413 extend from the insert 407 to the insert 411. The compression coils 413 may be configured to bypass load in length between a distal primary flex point and a proximal secondary flex point. The compression coils 413 facilitate independent flex planes so that both planes of flex do not activate when one plane of flex is desired to flex. The compression coils 413 may allow for the proximally slotted hypotube section 406P to retain rigidity for specific bending of the distally slotted hypotube section 406D. The compression coils 413 may isolate force so only the primary flex is flexed.

For the pair of proximal pull wires 410B, the wires can be spaced approximately 180° from one another to allow for steering in both directions. Similarly, if a pair of distal pull wires 410A is used, the wires can be spaced approximately 180° from one another to allow for steering in both directions. In some embodiments, the pair of distal pull wires 410A and the pair of proximal pull wires 410B can be spaced approximately 90° from each other. Opposing wires could be used to provide anti-flex mechanism. In some embodiments, the pair of distal pull wires 410A and the pair of proximal pull wires 410B can be spaced approximately 0° from each other. However, other locations for the pull wires can be used as well, and the particular location of the pull wires is not limiting. In some embodiments, the distal pull wire 410A can pass through a lumen attached within the lumen of the rail hypotube 406. This can prevent an axial force on the distal pull wire 410A from creating a bend in a proximal section of the rail hypotube 406. The rail subassembly 21 is disposed so as to be slidable (e.g., translatable) over the radially inner subassemblies. As the rail hypotube 406 is bent, it presses against the other subassemblies to bend them as well, and thus the other subassemblies of the delivery device 15 can be configured to steer along with the rail subassembly 21 as a cooperating single unit, thus providing for full steerability of the distal end of the delivery device 15. Additional structural and operation details of a rail subassembly, such as those described in connection with rail assemblies in U.S. Publication No. 2019/0008640 and U.S. Publication No. 2019/0008639, which are hereby incorporated by reference herein, may be incorporated into the rail subassembly 21.

FIG. 4C schematically illustrates how an outer compression coil 413A and proximal pull wire 410B1 can have a longer length than an inner compression coil 413B and proximal pull wire 410B2 of the rail subassembly 21 so that they don't occupy the same space, to reduce lumen obstruction during bending, and/or to facilitate ease of bending in one direction.

FIG. 4D-2 schematically illustrates a method of manufacturing that comprises thru-wall welding performed during manufacture of the rail subassembly (as compared to prior direct wire welding techniques). FIG. 4D-1 illustrates a prior art welding technique and FIG. 4D-2 illustrates an embodiment of a thru-wall welding technique. The thru-wall welding technique may advantageously be used to weld the pull wires 410 to the inserts (e.g., insert 407, 411 tip 408) within a lumen of the rail hypotube 406. In accordance with several embodiments, thru-wall welding advantageously does not comprise welding directly to the pull wires 410. Welding directly to the wires 410 (as is shown in FIG. 4D-1) can cause annealing and embrittling of a majority or of an entirety of a circumference of a pull wire (which has hard temper for strength) if heated too much. With reference to FIG. 4D-2, thru-wall welding can involve intentionally forming through-holes in between an outer diameter and inner diameter of a wall of a lumen and controlling a wall thickness to facilitate thru-wall welding in a manner that penetrates the hypotube or lumen wall but limits circumferential extent of heating of the pull wire (e.g., less than 20% of circumference, less than 25% of circumference, less than 30% of circumference). In some embodiments, thru-wire welding allows for welding along a single line (e.g., a line extending between the pull wires) instead of along multiple lines (e.g., one line for each pull wire).

### Mid-Shaft Subassembly

Moving radially inwardly, the next subassembly is the mid-shaft subassembly 22. FIG. 5A shows a perspective view of the mid-shaft subassembly 22 of the delivery device 15 of the delivery system 10. FIG. 5B shows a side view of the mid-shaft subassembly 22. The mid-shaft subassembly 22 can include a distal mid-shaft hypotube 502 generally attached at its proximal end (e.g., via laser welding or a heat shrink connector) to a mid-shaft proximal tube 504, which in turn can be attached at its proximal end to the handle 14 (e.g., via mid-shaft adapter 505), and a distal outer retention member or pusher 506 located at the distal end of the mid-shaft hypotube 502. These components of the mid-shaft subassembly 22 can form a lumen (e.g., middle lumen) for other inner subassemblies to pass through.

The mid-shaft subassembly 22 can be located within a lumen (e.g., rail lumen) of the rail subassembly 21. The mid-shaft hypotube 502 can be formed of a metallic alloy (e.g., cobalt chrome, nickel-chromium-cobalt alloy, nickel-cobalt base alloy, nickel-titanium alloy, stainless steel and titanium). The mid-shaft hypotube 502 may comprise an interrupted spiral cut pattern. In alternative embodiments, the mid-shaft hypotube 502 comprises a longitudinally pre-compressed high density polyethylene (HDPE) tube. FIG. 5A shows a similar view as FIG. 4A, but with the rail subassembly 21 removed, thereby exposing the mid-shaft subassembly 22.

Similar to the other subassemblies, the mid-shaft hypotube 502 and/or mid-shaft proximal tube 504 can comprise a tube or lumen, such as a hypodermic tube or hypotube (not shown). The tubes can be made from one of any number of different materials including Nitinol, stainless steel, and medical grade plastics. The tubes can be a single piece tube or multiple pieces connected together. Using a tube made of multiple pieces can allow the tube to provide different characteristics along different sections of the tube, such as rigidity and flexibility. The mid-shaft hypotube 502 can be a metal hypotube. The mid-shaft hypotube 502 can have a number of slots/apertures cut into the hypotube. In some embodiments, the cut pattern can be the same throughout. In some embodiments, the mid-shaft hypotube 502 can have different sections having different cut patterns. The mid-shaft hypotube 502 can be covered or encapsulated with a layer of ePTFE, PTFE, or other material so that the outer surface of the mid-shaft hypotube 502 is generally smooth. At least a portion of a length of the mid-shaft proximal tube 504 may be covered with a heat shrink tubing or wrap.

The pusher 506 may be configured for radially retaining a portion of the implant (e.g., prosthesis) 30 in a compacted configuration, such as a proximal end of the implant 30. For example, the pusher 506 may be a ring or covering that is configured to radially cover a proximal end portion (e.g., suture eyelets portion or proximal-most inflow portion) of the implant 30.

FIGS. 5B-1 to 5B-3 illustrate an embodiment of a distal pusher 506 of a mid-shaft subassembly 22, in which FIG. 5B-3 is a cross sectional view of the line 5B-3-5B-3 of FIG. 5B-2. FIGS. 5B-4 to 5B-6 illustrate another embodiment of a distal pusher 506A of the mid-shaft subassembly 22, in which FIG. 5B-6 is a cross sectional view of the line 5B-6-5B-6 of FIG. 5B-5. A distal pusher 506 of FIGS. 5B-1 to 5B-3 and a distal pusher 506A of FIGS. 5B-4 to 5B-6 have substantially the same outer and inner diameters Φ1 and Φ2 forming a cylindrical shape when viewed from the top. However, the distal pusher 506A does not have a lip and cup portion 507 having a height H7, thus having a flat top surface 509, compared to the distal pusher 506 having a thin wall having a radius of curvature R1 at the upper surface. Therefore, a total height H6 of the distal pusher 506 of FIGS. 5B-1 to 5B-3 is reduced by about a height H7. Further, removal of the material comprising the lip and cup portion 507 may leave only a flat surface to oppose an inflow side of the valve prosthesis during capsule retraction for valve deployment. The distal pusher 506A may have increased room (e.g., increased cross-sectional area) to fit the inflow struts of the outer frame 34 against the inside of the pusher 506A. The distal pusher 506A may also provide reduced docking forces (e.g., about 50% reduction in docking force compared to the distal pusher 506) as the suture portions attached to the proximal-most or inflow struts tension the outer frame 34 against the flat pusher surface without any lip or bump to pull the eyelets 35 over.

FIG. 5C shows a side cross-section view showing a close-up view of a distal end portion of the mid-shaft subassembly 22, which shows the proximal end portion (e.g., proximal-most portion, or just the suture eyelets 35) of the implant 30 being retained within the pusher 506. The pusher 506 can also be considered to be part of the implant retention area 316 and may be at the proximal end of the implant retention area 316. The pusher 506 may comprise a frustoconical or cup shape that is riveted or fastened on its opposite sides to the distal end of the mid-shaft hypotube 502. The pusher 506 may be formed of PEEK material, ferrous material, platinum iridium, or other fluorogenic material to facilitate radiographic imaging. The pusher 506 may also be formed of other thermoplastic, polymeric, or metallic materials. The pusher 506 may be loaded with radiopaque material (e.g., 5-40% barium sulfate loading) to facilitate detection (e.g., made fluorogenic) under radiographic imaging (e.g., fluoroscopy). The mid-shaft subassembly 22 may be disposed so as to be individually slidable (e.g., translatable) with respect to the other subassemblies. The mid-shaft adapter 505 operably couples to the depth knob 920 to effect ventricular/atrial movement within a heart (e.g., for implementations in which the implant 30 is a mitral or tricuspid replacement heart valve). Additional structural and operational details of a mid-shaft subassembly 22, such as those described in connection with mid assemblies in U.S. Publication No. 2019/0008640 and U.S. Publication No. 2019/0008639, which are hereby incorporated by reference herein, may be incorporated into the mid-shaft subassembly 22.

### Release and Manifold Subassemblies

In accordance with several configurations, a delivery device includes a suture-based release mechanism that includes a plurality of suture portions that are only coupled to a distal end portion of the delivery device and no not extend along the delivery device to a proximal handle that controls operation of the suture-based release mechanism. A first end of each of the plurality of suture portions may be fixedly attached to the distal end portion of the delivery device and a second end of each of the plurality of suture portions are releasably attached to the distal end portion of the delivery device after being inserted through a retention member (e.g., opening or eyelet) of an implant (e.g., replacement heart valve). The suture portions may be released (e.g., decoupled) from the implant by operator actuation of an actuator on a handle of the delivery device.

The suture-based release mechanism may include dual coaxial sliding shafts, or lumens. It should be appreciated that reference to lumens in the disclosure may be referring to shafts or tubes comprising lumens. The dual coaxial sliding shafts may be operably coupled to the actuator on the handle of the delivery device. The first end of each of the plurality of suture portions may be fixedly attached to a distal tip of an inner lumen of the dual coaxial sliding shafts. The second end of each of the plurality of suture portions may be releasably coupled to one or more retention members of the distal end portion of the inner shaft. Translation of the outer shaft with respect to the inner shaft of the dual coaxial sliding shafts or lumens by actuation of the actuator on the handle may cause the suture portions to be disengaged or decoupled from the one or more retention members of the distal end portion of the inner shaft.

Moving radially inward from the mid-shaft subassembly 22, FIG. 6A shows a perspective view of a release subassembly 23 of the delivery device 15 of the delivery system 10. FIG. 6B shows a side cross-section view of the release subassembly 23 of FIG. 6A. The release subassembly 23 operates in conjunction with the manifold subassembly 24 to facilitate retention and release of the implant or prosthesis 30. The release subassembly 23 extends through a central lumen of the mid-shaft subassembly 22. The release subassembly 23 includes a release shaft 602 that includes a lumen. The manifold subassembly 24 extends through the lumen of the release subassembly 23. The mid-shaft subassembly 22 acts as a compression member backstop and the manifold subassembly 24 acts as the tension member such that the mid-shaft subassembly 22 prevents retreating of the implant 30 when the capsule subassembly 306 is pulled back and the manifold subassembly prevents deployment/expansion of the implant 30 (or distal movement of the implant 30).

The distal portion of the release shaft 602 may include laser cut portions having various spine patterns. For example, a distal-most portion (e.g., ˜1 cm) of the release shaft 602 may include a dual spine laser cut pattern and a portion proximal of the distal-most portion (e.g., ˜5 cm proximal of the distal-most portion) may include a universal laser cut spine pattern. The dual spine pattern portion may only travel through the primary distal flex portion of the rail hypotube 406 and the universal spine pattern portion may travel through both the primary and secondary flex portions of the rail hypotube 406. At least a portion of a length of the release shaft 602 may be surrounded by a heat-shrink wrap or liner. The proximal end of the release shaft 602 is operably coupled to the handle 14 (e.g., via release adapter 604). The release subassembly 23 also includes a distal release tip 605 coupled to a distal end of the release shaft 602 via coupler 607, which may be formed of PEBAX or other thermoplastic elastomer material. The distal release tip 605 may be welded to the distal end of the release shaft 602. The release adapter 604 includes release snaps 606 on opposite lateral sides. The release snaps 606 engage with a distal potion of the manifold adapter 704 after release of the tethers or sutures so as to prevent movement of the manifold subassembly 24 and release subassembly 23 with respect to each other, which could cause the windows 610 of the distal release tip 605 to close and inadvertently retain one of the sutures or tethers. Thus, the release snaps 606 convert the release/manifold mechanism from a normally-closed configuration to an open configuration and allows the manifold subassembly 24 and release subassembly 23 to track proximally together. The release subassembly 23 further includes a release spring 608 that extends between the release adapter 604 and a location within a manifold adapter 704 of the manifold subassembly 24.

FIGS. 6C, 6D, and 6E show a close-up, side view, side cross-section view, and bottom view, respectively, of the distal release tip 605. The distal release tip 605 cooperates in conjunction with a distal end portion of the manifold subassembly 24 to facilitate prevention of premature release of the implant 30 and to facilitate release (e.g., untethering) of the implant 30 when ready for final implantation. The distal release tip 605 includes three windows 610 spaced apart around a circumference of the distal release tip 605 and three slots 612, with each slot 612 positioned between two adjacent windows 610. The windows 610 may be laser cut into the distal release tip 605. The three windows 610 may be equally spaced apart circumferentially and the slots 612 may be positioned equally circumferentially between adjacent windows 610. A distal end of each of the slots 612 includes an inwardly-protruding retention member 614 (e.g., tab, protrusion, lock, anchor). The inwardly-protruding tabs 614 are adapted to be aligned with and extend within corresponding slots of the manifold subassembly 24 so as to control axial movement (e.g., to provide positive datums for distal and proximal travel) and to prevent rotation of the release subassembly 23 with respect to the manifold subassembly 24, as will be described in more detail below.

Moving radially inward, FIG. 7A shows a perspective view of the manifold subassembly 24 of the delivery device 15. FIG. 7B shows a side cross-section view of the manifold subassembly 24 of FIG. 7A. The manifold subassembly 24 extends through and along the lumen of the release subassembly 23. The manifold subassembly 24 includes a proximal subassembly 701 and a distal subassembly 703. The proximal subassembly 701 includes a proximal shaft 702 having a proximal end that extends into the handle 14 of the delivery device 15 and is operably coupled to the handle 14 via a manifold adapter 704. The proximal shaft 702 may be coupled to the distal subassembly 703 by a manifold cable 705. The manifold cable 705 may comprise a multi-layer cable comprised of two, three, four, five or more layers. In some implementations, the manifold cable 705 comprises a tri-layer cable in which two outer layers function for tension and act together to prevent unwrapping of the outer layers and an inner layer comprises a single-filar coil that provides compression and prevents collapse. In some implementations, each layer is wound in an opposite direction as the adjacent layer (e.g., clockwise, counter-clockwise, clockwise or counter-clockwise, clockwise, counter-clockwise). The wire size, wire tension, pitch, number of filars in each layer, material, and material properties may vary. An inner coil may comprise one to ten filars closely wound with a 0 to 0.005″ gap. The middle and outer coils may each comprise one to ten filars and be closely wound with a 0 to 0.010″ gap. The manifold cable 705 may be formed of one or more materials, including, for example, nitinol, ferrous material such as stainless steel, and/or cobalt chrome material. The temper (e.g., strength) of the wires may range from 100 KSI to 420 KSI (kip/square inch) and an ultimate tensile strength of the manifold cable 705 may be greater than 110 pounds of force. The cross-section of the wires may be flat or round. The tri-layer cable may be configured to prevent diameter change during stretching. In other implementations, the proximal shaft 702 extends all the way to and is bonded with a proximal end of the distal subassembly 703.

FIG. 7C shows a close-up view of the distal subassembly 703 of the manifold subassembly 24. FIG. 7D shows a bottom view of the distal subassembly 703 of the manifold subassembly 24. As shown, the distal subassembly 703 includes a proximal tether retention component 706 and a distal tether retention component 707. The distal tether retention component 707 may be coupled (e.g., permanently bonded, welded) to a distal end of the proximal tether retention component 706. As shown best in FIG. 7D, the distal tether retention component 707 may comprise a cog that includes outwardly-extending tether cleats 708 circumferentially spaced around the cog. Openings or gaps 709 exist between adjacent tether cleats 708 to receive portions of the tether or suture 710. The distal tether retention component 707 may be formed of metal through an electrical discharge machining process. The proximal tether retention component 706 may also be formed of metal and formed via a laser cutting or electrical discharge machining process. The distal tether retention component 707 may include proximal and distal seal members 711, 713 (e.g., retention rings) that are sealed (e.g., welded, glued or otherwise adhered) to opposite upper and lower sides of the distal tether retention component 707 during manufacture to seal off the openings or gaps 709 between the tether cleats 708 so as to prevent the tether or suture 710 from being removed or uncoupled from the distal tether retention component 707. In accordance with several embodiments, the tether 710 is intended to be permanently coupled to (i.e., non-removable from) the distal tether retention component 707. The number of tether cleats 708 may correspond to the number of eyelets on the implant 30 (e.g., upper eyelets of the outer frame 34). The number of tether cleats 708 is nine in the illustrated embodiment; however, other numbers of tether cleats 708 may be used.

The tether or suture 710 may be a continuous piece of tether or suture that forms offset proximal loops and distal loops along its continuous length upon assembly during manufacturing. The proximal loops are wrapped around the tether cleats 708 and the distal loops are fed through a respective eyelet on a proximal end of the implant or prosthesis 30 (e.g., upper eyelet of an outer frame 34) and then removably coupled to the delivery device 15 (e.g., the proximal tether retention component 706 of the manifold subassembly 24).

During assembly, the continuous tether or suture 710 may be coupled to the distal tether retention component 707 according to the following example implementation. One end of the continuous tether or suture 710 may start at a location spaced distal to the distal tether retention component 707. With the one end remaining there, the tether 710 is then wrapped around a first tether cleat 708 and then fed back through an opening or gap 709 on the other side of the first tether cleat 708 to form a first proximal loop and then brought back to a location spaced distal to the distal tether retention component 707 to start formation of a first distal loop. The process is repeated for each of the tether cleats 708 until all of the proximal and distal loops are formed and the second end of the continuous tether 710 is brought near the first end of the continuous tether 710 and the two ends are knotted together and bonded to form a single continuous strand. The tether assembly process may be facilitated by an assembly component that can be placed at an appropriate spacing distance distal of the distal tether retention component 707 and that includes pegs around which portions of the continuous tether 710 can be wrapped to form the distal loops at uniformly-spaced distances from the distal tether retention component 707. The proximal loops may be prevented from unhooking from the tether cleats 708 by the proximal and distal seal members 711, 713. The continuous tether 710 may comprise ultra-high-molecular-weight polyethylene (UMHWPE) force fiber suture, an aramid suture, or an aramid and UMHWPE blend suture material. In some embodiments, aramid material may advantageously bond and prevent floss and/or fretting failure due to asymmetric loading of the suture during detachment. In accordance with several embodiments, the continuous tether 710 advantageously does not run an entire length of the delivery device or system (e.g., all the way to the handle) because elongation at load would be significant and any mechanism added to compensate could add increased complexity and could potentially be unreliable and/or not user-friendly.

FIG. 7E shows a flat cut pattern of the proximal tether retention component 706 of the distal subassembly 703. As shown, the proximal portion of the proximal tether retention component 706 comprises a dual spine laser cut pattern. The dual spine laser cut pattern of the proximal tether retention component 706 may match a dual spine laser cut pattern of the rail subassembly 21 and the release subassembly 23. The distal end portion of the proximal tether retention component 706 comprises three circumferentially spaced slots 714 and three openings or windows 715. The slots 714 are configured to align circumferentially with the slots 612 of the distal release tip 605 and the openings or windows 715 are configured to align circumferentially with the windows 610 of the distal release tip 605. Other numbers of slots 714 and openings 715 (e.g., two, four, five, six, seven, eight, nine) may also be used in other embodiments. Each opening 715 includes a tab, finger, or peg, 716 extending a certain distance into a respective opening 715 from a distal edge of the respective opening 715. A length of each tab 716 is sufficient such that one or more distal tether loops can be looped over a top (or proximal end) of a respective tab 716 and pushed distally so as to retain the one or more distal tether loops. As shown, the three tabs 716 each have a different length in order to facilitate the initial tether assembly process. However, in other configurations, the three tabs 716 may have an equal or substantially equal length. Each tab 716 may receive one or more distal tether loops. In one implementation where there are nine distal tether loops, each tab 716 may retain three distal tether loops. The slots 714 may be equally circumferentially spaced around a longitudinal axis of the proximal tether retention component 706 and may be sized and spaced so as to align with corresponding slots 612 of the release subassembly 23 so as to receive a respective inwardly-protruding retention member 614.

### Operation of the Suture-Release Mechanism

FIGS. 8A and 8B show distal end portions of the release and manifold subassemblies in a locked configuration and unlocked configuration, respectively. The locked configuration shown in FIG. 8A is the default configuration after assembly. The release and manifold subassemblies are intended to remain in the locked configuration until a clinician has determined that the implant 30 is in a final desired implantation location. In the locked configuration, the proximal ends of the tabs 716 are positioned proximal of the proximal edge of the release windows 610 such that the distal tether loop(s) wrapped around the tabs 716 cannot be unhooked from the tabs 716, which could cause premature release of the tether 710. For simplicity and to avoid confusion in the figure, only one distal tether loop is shown wrapped around one of the tabs 716; however, two, three, or more tether loops may be hooked onto, or wrapped around, each of the tabs 716. The spring 608 shown in FIG. 6A (which is biased in a compressed configuration) keeps the release adapter 604 and the manifold adapter 704 apart and forces the release subassembly 23 distal in compression so that the release subassembly 23 and the manifold subassembly 24 do not move longitudinally with respect to each other, thereby keeping the release subassembly 23 and the manifold subassembly 24 in the locked configuration shown in FIG. 8A until an operator is ready to release the suture(s) or tether(s). As discussed in connection with FIGS. 9A and 9B, a safety member (e.g., pin) 927 of the handle also prevents the manifold subassembly 24 from moving distally out of the locked configuration until ready.

Once the clinician has determined that the implant 30 is in a final desired implantation position and all verification processes have been performed and confirmed, the safety member 927 is removed from the handle and the spring 608 is placed even more in compression. As the release knob 925 is rotated distally, the spring 608 is compressed further and pushes the manifold subassembly 24 distally out of the release subassembly 23 into the unlocked configuration shown in FIG. 8B. As shown in FIG. 8B, the manifold subassembly 24 has been pushed distally enough with respect to the release subassembly 23 that the proximal end of at least one of the tabs 716 is within the release window 610 such that a distal tether loop of the tether 710 can be unhooked from the tab 716, especially upon continued distal advancement of the manifold subassembly 24. FIG. 8C illustrates how one of the tether or suture loops transitions from being tethered to being untethered, or released, as the release and manifold subassemblies effect transition between a locked configuration and an unlocked configuration. Also as shown in FIG. 8C, the corresponding slots 612 and 714 are aligned so as to prevent rotation of the manifold subassembly 24 with respect to the release subassembly 23 (due to inwardly-protruding retention members 614), thereby retaining alignment of the tabs 716 within the windows 610 of the release subassembly 23. FIG. 8D shows an implant 30 fully tethered between eyelets on a proximal end of the implant (e.g., upper eyelet of an outer frame 34 of a valve prosthesis 30) and the manifold subassembly 24 of the delivery device 15. As shown, there are nine tether loops or portions connected to nine eyelets; however, the number may vary as desired and/or required. The suture or tether retention mechanism described in connection with FIGS. 8A-8D advantageously does not require the tethers or sutures 710 to extend through and along a long portion of the length of the delivery device 15 (e.g., to a proximal handle 14), thereby advantageously preventing or reducing the likelihood of snagging or catching of the suture or tether portions on intervening components within the delivery device, preventing or reducing the likelihood of tangling of suture or tether portions due to decreased lengths, reducing complexity of operation required by an operator to release a tether, simplifying assembly and manufacture, and/or reducing an amount of suture or tether material required. Instead, the suture or tether portions are advantageously only coupled to the distal end portion of the delivery device.

### Handle

FIG. 9A shows a perspective view of the handle 14 of the delivery device 15. FIG. 9B shows a side cross-section view of the handle 14. The handle 14 includes multiple actuators, such as rotatable knobs, that can manipulate different components (e.g., cause movement of respective subassemblies of the shaft assembly 12) of the delivery system 10. The distal end of the handle 14 includes a capsule knob 905. Rotation of the capsule knob 905 in one direction causes proximal movement of the outer sheath subassembly 20 in an axial direction so as to unsheathe and deploy a distal portion (e.g., ventricular portion) of the implant 30 from the capsule subassembly 306. Rotation of the capsule knob 905 in the opposite direction causes distal movement of the outer sheath subassembly 20 (including the capsule subassembly 306) so as to recapture, retrieve, or resheath, the implant 30 within the capsule subassembly 306. The outer sheath subassembly 20 may be individually translated with respect to the other subassemblies in the delivery device 15. With reference back to FIG. 5C, the distal end of the implant 30 can be released first, while the proximal end (e.g., proximal-most eyelets 35 but not a proximal circumferential shoulder of an outer frame) of the implant 30 can remain radially compressed within the pusher 506 of the mid-shaft subassembly 22. Because the capsule assembly 306 is so robust and provides both tension and compression strength, only the proximal-most portion of the implant 30 (e.g., the eyelets 35) need to be retained by the pusher 506 and the pusher 506 can be relatively short in length. The tethers 710 and release subassembly 23 and manifold subassembly 24 also remain within the mid-shaft subassembly 22 until rotation of a release knob 925.

Moving proximally, the handle 14 includes a stabilizer mounting area 910 adapted to interface with a clamp of a stabilizer assembly 1100 configured to control the medial/lateral position of the delivery device 15. Moving further proximally are the primary flex rail knob 915A and the secondary flex rail knob 915B. Rotation of the primary flex rail knob 915A causes flexing of the primary flex portion, or distal slotted hypotube section 406D of the rail hypotube 406 to effect changes in medial/lateral trajectory. Rotation of the secondary flex rail knob 915B causes flexing of the primary flex portion, or proximal slotted hypotube section 406P of the rail hypotube 406 to effect changes in anterior/posterior trajectory. However, the number of flex rail knobs 915 can vary depending on the number of pull wires used.

Proximal to the secondary flex rail knob 915B is a depth knob 920 that, in some embodiments, controls simultaneous movement of the outer assembly 20, mid-shaft subassembly 22, release subassembly 23, manifold subassembly 24, and nose cone subassembly either distal or proximal (thereby moving the delivery device 15 ventricular or atrial for a mitral valve or tricuspid valve implantation). The depth knob 920 may move the subassemblies together relative to the rail subassembly 21. Further proximal is the release knob 925 (sometimes also referred to as the manifold knob since it controls simultaneous longitudinal movement of both the release subassembly 23 and the manifold subassembly 24 until the release subassembly 23 encounters a hard stop member within the handle 14 and then only the manifold subassembly 24 continues to move longitudinally in a distal direction with respect to the release subassembly 23). The release knob 925 may be rotated proximally to put tension on the manifold subassembly 24 during loading or during recapture, or retrieval, of the implant 30. The release knob 925 may be rotated distally to deploy the proximal portion (e.g., atrial portion) of the implant 30 after the capsule subassembly 306 has been retracted to deploy the distal portion (e.g., ventricular portion) of the implant 30. Distal movement of the release knob 925 takes tension off the manifold subassembly 24. As discussed above, the safety stop member 927 prevents the release knob 925 from moving distally enough to allow release of the implant 30 until the safety stop member 927 is removed from the handle 14. Once the safety stop member 927 has been removed, continued distal movement of the release knob 925 causes the manifold subassembly 24 to move distally relative to the release subassembly 23 (after the release subassembly 23 abuts against a mechanical stop member within the handle 14 that prevents further distal movement of the release subassembly 23) to facilitate release of the tether 710 from the manifold subassembly 24 (e.g., the distal tether loops are allowed to be pushed off of the tabs 716 of the proximal tether retention member 706 of the manifold subassembly 24 by the windows 610 of the release subassembly 23). The proximal-most knob is the nose cone knob 930, rotation of which causes proximal and distal movement of the nose cone subassembly.

### Nose Cone Subassembly

The nose cone subassembly is the most radially-inward subassembly and may include a nose cone shaft having a distal end connected to a nose cone 87 (labeled in FIG. 14C). For example, the knob 930 can be a portion of the nose cone subassembly that extends from a proximal end of the handle 14. Thus, a user can rotate the knob 930 to translate the nose cone shaft distally or proximally individually with respect to the other shafts. This can be advantageous for proximally translating the nose cone 87 into the outer sheath assembly 20/capsule subassembly 306, thus facilitating withdraw of the delivery device 15 from the patient. The nose cone 87 can have a tapered tip. The nose cone 87 can be made of a thermoplastic or elastomer (e.g., PEBAX or polyurethane) for atraumatic entry and to minimize injury to venous vasculature. The nose cone 87 can also be radiopaque to provide for visibility under fluoroscopy. The nose cone assembly is preferably located within a lumen of the manifold subassembly 24. The nose cone assembly can include a lumen for a guide wire to pass therethrough. Additional structural and operation details of a handle and a nose cone assembly, such as those described in connection with handles and nose cone assemblies in U.S. Publication No. 2019/0008640 and U.S. Publication No. 2019/0008639, which are hereby incorporated by reference herein, may be incorporated into the handle 14 and nose cone subassembly herein.

### Introducer Assembly

FIG. 10 shows components of an introducer assembly 1000 of the delivery system 10. The introducer assembly 1000 includes an introducer sheath 1005, a dilator 1010, an introducer 1012, and a loader 1015. The dilator 1010 helps to dilate the vasculature for insertion of the delivery device 15 and/or introducer sheath 1005. The dilator 1010 may be removed and replaced with additional dilators (e.g., dilators of differing diameters) if desired and/or required. After removal of the dilator 1010, the introducer 1012 (which may be inserted into and advanced along the introducer sheath 1005 so that a tapered distal tip of the introducer 1012 extends beyond an open distal end of the introducer sheath 1005) and the introducer sheath 1005 are advanced together into the dilated vasculature through an incision. For a transfemoral delivery approach, the vasculature is a femoral vein within a leg of the subject. The introducer sheath 1005 may include a side portion to facilitate heparinized saline or other flushing fluid. The introducer sheath 1005 may be configured to remain stationary with respect to the leg of the subject. The loader 1015 is adapted to be inserted into the proximal end of the introducer sheath 1005 in order to open up aggressive one-way valves in the introducer sheath 1005 prior to insertion of the delivery device 15 to make insertion of the delivery device 15 through the introducer sheath 1005 easier. The loader 1015 may also advantageously reduce friction between the delivery device 15 and the introducer sheath 1005 while the delivery device 15 is inserted and while the delivery device 15 is manipulated during an implantation procedure. In some implementations, the introducer 1012 and introducer sheath 1005 may not be used and the delivery device 15 may be inserted directly into the dilated vasculature.

### Stabilizer Assembly

FIG. 11 illustrates how the handle 14 of the delivery device 15 interfaces with an embodiment of the stabilizer assembly 1100 of the delivery system 10. FIG. 11A shows a perspective view of the stabilizer assembly 1100 without the delivery device 15 attached. FIG. 11B shows a top view of the stabilizer assembly 1100 of FIG. 11A. The stabilizer assembly 1100 includes a clamp 1105, a guide assembly 1110, a rail 1115, and a base 1120. The clamp 1105 is configured to couple to the stabilizer mounting area 910 of the handle 14 of the delivery device 15. The guide assembly 1110 is configured to cause changes in the medial/lateral position of the delivery device 15 by movement along the rail 1115. The rail 1115 may be mounted on and secured to the base 1120. Additional details regarding the stabilizer assembly 1100 may be found in US Pat. Publ. No. 2020/0108225 published on Jan. 10, 2020, the entire contents of which are incorporated by reference herein.

### Delivery Methods

FIG. 12 illustrates a schematic representation of a transseptal delivery approach. As shown in FIG. 12, in one embodiment the delivery system 10 can be placed in the ipsilateral femoral vein 1074 and advanced toward the right atrium 1076. A transseptal puncture using known techniques can then be performed to obtain access to the left atrium 1078. The delivery system 10 can then be advanced in to the left atrium 1078 and then to the left ventricle 1080. FIG. 12 shows the delivery system 10 extending from the ipsilateral femoral vein 1074 to the left atrium 1078. In embodiments of the disclosure, a guide wire is not necessary to position the delivery system 10 in the proper position, although in other embodiments, one or more guide wires may be used.

Accordingly, it can be advantageous for a user to be able to steer the delivery system 10 through the complex areas of the heart in order to position a replacement mitral valve in line with the native mitral valve. This task can be performed with or without the use of a guide wire with the above disclosed system. The distal end of the delivery system 10 can be advanced into the left atrium 1078. A user can then manipulate the rail subassembly 21 to target the distal end of the delivery system 10 to the appropriate area. A user can then continue to pass the bent delivery system 10 through the transseptal puncture and into the left atrium 1078. A user can then further manipulate the delivery system 10 to create an even greater bend in the rail subassembly 21. Further, a user can torque the entire delivery system 10 to further manipulate and control the position of the delivery system 10. In the fully bent configuration, a user can then place the replacement valve in the proper location. This can advantageously allow delivery of a replacement valve to an in-situ implantation site, such as a native mitral valve, via a wider variety of approaches, such as a transseptal approach.

FIG. 13 illustrates a schematic representation of a portion of an embodiment of a replacement heart valve (implant 30) positioned within a native mitral valve of a heart 83. Further details regarding how the implant 30 may be positioned at the native mitral valve are described in U.S. Pat. Pub No. 2015/032800 published on Nov. 19, 2005, the entirety of which is hereby incorporated by reference, including but not limited to FIGS. 13A-15 and paragraphs [0036]-[0045]. A portion of the native mitral valve is shown schematically and represents typical anatomy, including a left atrium 1078 positioned above an annulus 1106 and a left ventricle 1080 positioned below the annulus 1106. The left atrium 1078 and left ventricle 1080 communicate with one another through the annulus 1106. Also shown schematically in FIG. 13 is a native mitral leaflet 1108 having chordae tendineae 1111 that connect a downstream end of the mitral leaflet 1108 to the papillary muscle of the left ventricle 1080. The portion of the implant 30 disposed upstream of the annulus 1106 (toward the left atrium 1078) can be referred to as being positioned supra-annularly. The portion generally within the annulus 1106 is referred to as positioned intra-annularly. The portion downstream of the annulus 1106 is referred to as being positioned sub-annularly (toward the left ventricle 1080).

As illustrated in FIG. 13, the implant 30 can be positioned so that the ends or tips of the distal anchors 37 are on a ventricular side of the mitral annulus 1106. The distal anchors 37 can be positioned such that the ends or tips of the distal anchors 37 are on a ventricular side of the native leaflets beyond a location where chordae tendineae 1111 connect to free ends of the native leaflets. The distal anchors 37 may extend between at least some of the chordae tendineae 1111 and, in some situations can contact or engage a ventricular side of the annulus 1106. It is also contemplated that in some situations, the distal anchors 37 may not contact the annulus 1106, though the distal anchors 37 may still contact the native leaflet 1108. In some situations, the distal anchors 37 can contact tissue of the left ventricle 1080 beyond the annulus 1106 and/or a ventricular side of the leaflets 1108.

FIGS. 14A-14E illustrate operation of the delivery device 15 by showing various steps of deployment and implantation of the implant (e.g., replacement heart valve) 30 using the delivery device 15 described herein. FIGS. 14A-14E show the positioning of the various subassemblies of the delivery device 15 with respect to each other and with respect to the implant 30 at the various steps of the procedure. The subassemblies are shown in a side cross-section view to facilitate visualization of the various subassemblies. For sake of simplicity and illustration, various portions of the implant 30 (e.g., skirt assembly 38 and padding 39) are not shown. FIG. 14A illustrates the delivery device 15 at a time in an implantation procedure in which the replacement heart valve 30 is completely retained within the capsule subassembly 306 of the outer subassembly 20 in a compressed configuration. As shown, a proximal-most portion (e.g., eyelet portion) of the replacement heart valve 30 is retained within the pusher 506 of the mid-shaft subassembly 22 and the remainder of the replacement heart valve 30 is compressed by the capsule subassembly 306. With reference to FIG. 14B, the capsule subassembly 306 has been retracted proximally (e.g., toward a proximal handle 14 of the delivery device 15 by rotating capsule knob 905 of the handle 14) to a position such that the replacement heart valve 30 is no longer constrained by the capsule subassembly 306 and the replacement heart valve 30 has been allowed to partially self-expand. The proximal-most portion (e.g., eyelet portion) of the replacement heart valve 30 remains constrained in a compressed configuration by the pusher 506 of the mid-shaft subassembly 22 such that the entire replacement heart valve 30 is not yet fully deployed.

As can be appreciated, the deployment of the distal and mid portions of the replacement heart valve 30 may occur in stages over time and not in an immediate full deployment. For example, the distal anchors 37 of the inner frame 32 of a dual-frame structure may be deployed first prior to deployment of the outer frame 34 (e.g., while the outer frame 34 and mid portion of the inner frame 32 remain constrained within the capsule subassembly 306), such as shown for example, in FIG. 5C. The distal anchors 37 of the inner frame 32 may be positioned through chordae tendineae of a native heart valve (e.g., mitral valve) and/or subannularly so as to capture the native leaflets of the heart valve between the distal anchors 37 and a main body of the outer frame so as to keep the native leaflets in an open configuration and to anchor the replacement heart valve 30 as a whole. Such a configuration and position is shown in FIG. 14J.

With reference to FIG. 14C, the manifold subassembly 24 and the release subassembly 23 have been advanced distally by rotation of the release knob 925 (as discussed previously herein) while the mid-shaft subassembly 22 remains fixed such that the proximal-most portion (e.g., eyelet portion) of the replacement heart valve 30 is advanced distally out of the pusher 506 of the mid-shaft subassembly 22, thereby deploying the replacement heart valve 30 into a fully-expanded configuration. However, the replacement heart valve 30 still remains tethered to the manifold subassembly 24 by the tether(s) 710 because the manifold subassembly 24 and the release subassembly 23 are in the “locked” configuration, as described previously herein in connection with FIGS. 8A-8D.

With reference to FIG. 14D, the manifold subassembly 24 has been moved distally relative to the release subassembly 23 (to transition the release subassembly 23 and the manifold subassembly 24 into the unlocked configuration described in connection with FIGS. 8A-8D) and the suture loop ends of the tether(s) 710 that were previously coupled to the tabs 716 of the manifold subassembly 24 have been uncoupled or released. With reference to FIG. 14E, the manifold subassembly 24 and the release subassembly 23 are retracted proximally together until the free suture loop ends of the tether(s) 710 are pulled out of the proximal eyelets 35 of the replacement heart valve 30 and the delivery device 15 is then removed from the implantation location, thereby leaving the replacement heart valve 30 in its final implantation location. The manifold subassembly 24 and the release subassembly 23 may be retracted into the outer sheath subassembly 20 or the outer sheath subassembly 20 may be advanced to cover the distal ends of the manifold subassembly 24 and the release subassembly 23 prior to withdrawal of the delivery device 15. However, the distal ends of the manifold subassembly 24 and the release subassembly 23 may alternatively remain distal of (outside) the outer sheath subassembly 20 as the delivery device 15 is withdrawn.

FIGS. 14F-4K illustrate various steps of deployment and recapture of the implant (e.g., replacement heart valve) 30 using the delivery device 15 described herein. For sake of simplicity and illustration, only the inner frame 32 and outer frame 34 of the implant 30 is illustrated (e.g., skirt assembly 38 and padding 39 as shown in FIG. 2C is not shown). The capsule subassembly 306 advantageously facilitates recapture of the implant 30 after an initial deployment. FIG. 14F illustrates an initial deployment of the implant 30 from the delivery device 15. For example, the initial deployment may be within a mitral valve annulus following a transfemoral and/or transseptal approach. Note that the implant 30 remains tethered to the delivery device 15 upon initial full deployment of the implant 30 to a fully expanded configuration. In some instances, a clinician may decide after performing various tests (e.g., using various imaging modalities and measurements) that the initial deployment location is not ideal. For example, the ideal position may be more superior (e.g., toward the atrium) or inferior (e.g., toward the ventricle) of the initial deployment location. In order to prevent damage to the implant 30 and to the heart, the implant 30 may be recaptured prior to movement of the implant 30 to a new implantation location. Recapturing of the implant 30 may be performed by advancing the capsule subassembly 306 of the outer sheath subassembly 20 distally over the implant 30 to cause the implant 30 to transition to a compressed configuration. FIGS. 14G and 14H show various stages of recapturing of the implant 30. As shown in FIG. 14G, the capsule subassembly 306 has been advanced distally (e.g., by rotating capsule knob 905 in a first direction) to capture the proximal portion of the implant 30. FIG. 14H shows full recapture of the implant 30, with the capsule subassembly 306 being fully advanced distally (e.g., until contact with a nose cone 87 of the nose cone subassembly or until the implant 30 is fully retained within the capsule subassembly 306). The configuration of FIG. 14H corresponds to the configuration of FIG. 14F but within the heart location.

After movement of the distal end of the delivery device 15 to a new location, the capsule subassembly 306 of the outer sheath subassembly 20 can again be retracted proximally (e.g., by rotating capsule knob 905 in an opposite, second direction from the first direction) to unsheathe the distal portion of the implant 30 (e.g., at a new implantation location within a mitral valve annulus or tricuspid valve annulus), as shown in FIG. 14I. The manifold and release subassemblies 23, 24 may then be advanced distally together (e.g., by rotation of release knob 925) to deploy the proximal-most portion of the implant 30 (e.g., proximal eyelets, posts or struts) out of the pusher 506 of the mid-shaft subassembly 22, as shown in FIG. 14J. After confirmation that the fully-deployed implant 30 is in an ideal and proper final implantation location, the tether 710 (e.g., tether loop ends) may be caused to be released from the manifold subassembly 24 (as shown in FIG. 14K) by continued rotation of the release knob 925 so that the release knob 925 translates further distally until the release subassembly 23 encounters a physical stop member in the handle 14 and the manifold subassembly 24 continues to translate distally with respect to the release subassembly 24. The delivery device 15 can be retracted and removed from the heart and then from the vasculature and then from the subject altogether.

### Skirt Assembly and Methods of Manufacturing or Assembling

FIGS. 15A and 15B illustrate different views of a configuration of a fully-assembled implant (e.g., valve prosthesis) 1230 including a skirt assembly 1238 (shown in FIGS. 17A-17D) positioned between the frames 1232, 1234 (shown in FIGS. 16A and 16B) and padding 1239 surrounding the anchors 1237. The implant 1230 can be similar to the configuration of the implant 30 illustrated in and described in relation to FIGS. 2-2K-2. Reference numerals of the same or substantially the same features may share the same last two digits.

FIG. 15C shows a prosthetic leaflet stitched to an inner frame 32 of a dual-frame valve prosthesis (e.g., implant 30, 1230). The inner frame 32 of the dual-frame valve prosthesis may include a prosthetic valve assembly composed of a plurality of flexible leaflets 1108A arranged to collapse in a ti-leaflet arrangement and reinforcing strips 1108B for securing the plurality of prosthetic leaflets 1108A to the inner frame 32 and securing a cusp edge portion 1108C of each prosthetic leaflet 1108A to the first end portion of the reinforcing strip 1108B. The dual-frame valve prosthesis (e.g., implant 1230) may be implanted to replace any heart valve (e.g., mitral valve, tricuspid valve, aortic valve, pulmonic valve) and the inner frame 32 of the dual-frame valve prosthesis may be configured to have an “hourglass” profile or shape when in an expanded configuration, as described elsewhere herein. Although the prosthetic leaflet stitching and valve assembly implementations are generally described herein with reference to a dual-frame valve prosthesis, the leaflet stitching and valve assembly implementations may also be used for assembly/manufacturing of a single frame implant or implants with more than two frames (e.g., three or more frames). For example, aortic and pulmonic prosthetic valve implants may incorporate a single frame (e.g., single frame valve with an hourglass profile) instead of a dual frame.

FIG. 15D-1 to 15D-5 show double stitching applied to a prosthetic leaflet to securely attach to an inner frame of the dual-frame valve prosthesis; however, the double stitch line 1108D can be incorporated into stitching for any prosthetic valve (e.g., single frame or more than two frames) and not only dual-frame valve prostheses. The double stitch line 1108D can be seen in FIGS. 15D-1 to 15D-3 by following, or connecting, the two separate rows of dots in the figures. Methods of assembling prosthetic leaflets 1108A to other components of the dual-frame valve prosthesis (e.g., portions or components of a skirt assembly and/or frame assembly) include folding over portions of the prosthetic leaflet edges or cloth skirt edges so as to cover exposed suture portions and to prevent direct contact between suture portions or potentially abrasive skirt edges and the prosthetic leaflets (e.g., belly portions of the prosthetic leaflets). The skirt assembly (e.g. skirt assembly 1238, 1248) may include multiple skirt portions. For example, the skirt assembly may include a first portion that includes a double stitch line with pre-drilled laser holes configured to align with holes of a double stitch line of a prosthetic leaflet. In other implementations, there are no pre-drilled laser holes and the stitching is sewn through cloth or tissue free hand without pre-formed (e.g., laser-drilled) holes. The first portion may comprise a reinforcing cloth skirt strip 1248A adapted to facilitate attachment of the skirt assembly to the prosthetic leaflets. The skirt assembly may also include a main portion 1248B adapted to facilitate attachment to a frame structure. In some implementations, a first portion of the skirt assembly (e.g., reinforcing cloth skirt strip(s) 1248A) that is sutured to the prosthetic leaflet 1108A can be folded on itself (either outwardly or inwardly) so as to cover a first line of exposed sutures 1109A, thereby preventing contact of the leaflet 1108A with a potentially abrasive skirt edge formed by cutting of the reinforcing cloth skirt strip 1248A and also preventing any portion of the sutures 1109A, 1109B from contacting the leaflet 1108A, which contact could also cause abrasion over time. FIGS. 15D-4 and 15D-5 illustrate examples of different portions of the reinforcing cloth skirt strip 1248A of the skirt assembly being folded over itself to prevent exposure or contact of the sutures 1109 with the leaflet 1108A.

For example, a method of assembling the leaflet 1108A to a dual-frame valve structure includes securing at least a component or portion of the skirt assembly (e.g., skirt assembly 1238) to an inner frame via a first line of sutures 1109A using reinforcing strips (e.g., reinforcing strips 1248A); securing the leaflets 1108A to the reinforcing strips 1248A via the primary suture or first line of sutures 1109A; folding the reinforcing strips 1248A over the first line of sutures 1109A to cover them and then suturing the folded-over portion of the reinforcing strips 1248A of the skirt assembly with a second line of sutures (e.g., secondary sutures) 1109B parallel to and spaced apart from the first line of sutures 1109A, which also do not contact any portion of the leaflet 1108A. The primary sutures 1109A and secondary sutures 1109B create more than one stitch line 1108D (e.g., a double stitch line, or two stitch lines). Again, the method of assembly may be applied to a single frame valve structure in addition to a dual-frame valve structure.

With reference to FIGS. 15E-1 to 15E-4, the method of assembling the leaflets 1108A to the dual-frame valve structure (e.g., implant 30, 1230) may alternatively or additionally include folding a cusp edge portion or tab 1108C of the leaflets 1108A inwardly and applying sutures 1109 to secure the folded cusp edge portion or tab 1108C to the reinforcing cloth strips 1237A of the skirt assembly. In this implementation, neither the sutures 1109 nor the skirt assembly (e.g., reinforcing cloth strips 1237A) are in contact with a belly portion of the leaflets 1108A. Again, this method of assembling may be applied to a single frame valve structure as well.

In some implementations, a double stitch line 1108D can include a second stitch line at the cusp edge portion 1108C of each prosthetic leaflet 1108A where it is attached to other components of the dual-frame valve prosthesis, so as to increase the retention strength of the stitch line and more evenly distribute stress throughout valve opening and closing while adding minimal extra bulk. The folded cusp edge portion or tab 1108C being positioned between the cloth of the skirt assembly and the exposed suture portions advantageously acts as a barrier to prevent abrasion on leaflet bellies as the prosthetic valve opens and closes over time. The leaflets 1108 may be formed of bovine or porcine pericardial tissue (such as RESILIA® bovine pericardial tissue). The RESILIA bovine pericardial tissue may advantageously resist calcification.

FIG. 16A illustrates a configuration of the inner frame 1232 coupled to a prosthetic valve assembly 1231 comprising a plurality of prosthetic valve leaflets (not shown). FIG. 16B illustrates a configuration of the outer frame 1234. The inner frame 1232 can be similar to the configuration of the inner frame 32 and the outer frame 1234 can be similar to the configuration of the outer frame 34 illustrated in and described in relation to FIGS. 2-2K-2. Reference numerals of the same or substantially the same features may share the same last two digits.

FIGS. 17A-17D illustrate a configuration of the skirt assembly 1238. The skirt assembly 1238 can include a cloth material. For example, the skirt assembly 1238 can include a single, integral piece of cloth or multiple pieces of cloth coupled together. The skirt assembly 1238 can include a proximal, or inflow, portion 1238A, a middle, or intermediate, portion 1238B, and a distal, or outflow, portion 1238C.

As shown in FIG. 17B, the skirt assembly 1238 can include varying diameters. For example, the skirt assembly 1238 can include a plurality of diameters D1, D2, D3, D4, D5, D6, D7. In some configurations, the third diameter D3 can be the greatest diameter. In some configurations, the seventh diameter D7 can be the smallest diameter. The first, second, fourth, fifth, and sixth diameters D2, D3, D4, D5, D6 can be between the third diameter D3 and the seventh diameter D7. The plurality of diameters D1, D2, D3, D4, D5, D6, D7 can be the same diameter or each of the diameters can be different. In accordance with several implementations, the skirt assembly 1238 techniques described herein advantageously facilitate transitioning between varying diameters within one single piece of cloth without having to cut the cloth into multiple components. Advantageously, by having a skirt assembly 1238 as an integral component with varying diameters D1, D2, D3, D4, D5, D6, D7, the amount of cloth used can be reduced and the thickness of the skirt assembly 1238 can be reduced. By reducing the thickness of the skirt assembly 1238, the loading and retrieval forces exerted on the implant 1230 during delivery and retrieval can be reduced.

As shown in the illustrated configuration, the skirt assembly 1238 can include a plurality of portions or extensions 1240A, 1240C to vary the diameter of the skirt assembly 1238. For example, the middle portion 1238B can include a body portion 1240B, the inflow portion 1238A can include a plurality of proximal portions or extensions 1240A extending from the body portion 1240B, and the outflow portion 1238C can include a plurality of distal portions or extensions 1240C extending from the body portion 1240B. The proximal extensions 1240A can be configured to be positioned between the inner frame 1232 and the outer frame 1234. For example, the outer frame 1234 may include a plurality of openings 1234D (as shown in FIG. 16B) and the proximal extensions 1240A can be received by the plurality of openings 1234D such that the proximal extensions 1240A can be positioned between the inner and outer frames 1232, 1234. The body portion 1240B can be configured to be positioned exterior to the outer frame 1234 when the implant 1230 is assembled. The distal extensions 1240C can be configured to be positioned between the inner frame 1232 and the outer frame 1234 on the inflow side of the implant 1230. For example, the distal extensions 1240C can be inserted through the space distal to a distal edge of the outer frame 1234 such that the distal extensions 1240C can be positioned between the inner and outer frames 1232, 1234 on the outflow side of the implant 1230.

In the illustrated configuration, the skirt assembly 1238 has a plurality of trapezoidal portions 1240A, 1240C. In other configurations, the skirt assembly 1238 can include portions 1240A, 1240C having a square shape, a triangular shape, a circular shape, or any other suitable shape. The plurality of proximal extensions 1240A can include 18 proximal extensions 1240A. In other configurations, the plurality of proximal extensions 1240A can include any number of proximal extensions (e.g., less than or more than 18 proximal extensions). The plurality of distal extensions 1240C can include 9 distal extensions. In other configurations, the plurality of distal extensions 1240C can include any number of distal extensions (e.g., less than or more than 9 distal extensions).

As shown in FIG. 17C, the skirt assembly 1238 can include a plurality of features 1242A, 1242B, 1242C, 1242D, 1242E, 1242F, 1242G, 1242H configured to assist in the assembly of the skirt assembly 1238 and the implant 1230. For example, the plurality of features can include a plurality of tabs 1242A that can extend from one or more of the proximal extensions 1240A. The tabs 1242A can be configured to be positioned between the eyelets 1235 of the inner frame 1232 and the outer frame 1234. Advantageously, the tabs 1242A can prevent corrosion of the eyelets 1235. In the illustrated configuration, the plurality of tabs 1242A can include 9 tabs 1242A on alternating proximal extensions 1240A. In some configurations, the plurality of tabs 1242A can be on each of the proximal extensions 1240A or on fewer than half of the proximal extensions 1240A.

In some configurations, the plurality of features can include a keying feature 1242B. The keying feature 1242B can be positioned on one side of one or more of the proximal extensions 1240A. The keying feature 1242B can indicate which side of the proximal extensions 1240A should be positioned on top of adjacent proximal extensions 1240A when the skirt assembly 1238 is folded and sewed into the folded configuration, as further describe below in reference to FIG. 17D.

In some configurations, the plurality of features can include a plurality of holes 1242C in the distal extensions 1240C. For example, each of the distal extensions 1240C can include one or more holes 1242C. In the illustrated configurations, each distal extension 1240C has a single hole 1242C. The plurality of holes 1242C can allow blood to flow into the enclosed volume of the implant 1230 (e.g., the volume between the inner frame 1232 and prosthetic valve assembly 1231, and the outer frame 1234 and skirt assembly 1238). The plurality of holes 1242C can be sized such that blood can flow through the holes 1242C into the implant 1230 but the blood is prevented or restricted from flowing out of the implant 1230. The holes 1242C can be positioned between the anchors 1237 of the inner frame 1232 (shown in FIG. 16A) when the implant 1230 is assembled so that the anchors 1237 do not restrict the blood from through the holes 1242C. Moreover, the holes 1242C can assist the manufacturer in properly attaching the skirt assembly 1238 to the inner and outer frames 1232, 1234 by ensuring the holes 1242C are positioned between the anchors 1237.

In some configurations, the plurality of features can include at least one tapered section 1242D. The at least one tapered section 1242D can be positioned on the outside of the outer frame 1234. In some configurations, the at least one tapered section 1242D can include two tapered sections 1242D that can be sewn together when the implant 1230 is assembled.

In some configurations, the plurality of features can include first alignment features 1242E and second alignment features 1242F. The first alignment features 1242E can be positioned on at least one side of at least one distal extension 1240C and/or adjacent the hole(s) 1242C. In the illustrated configuration, each distal extension 1240C includes a pair of first alignment features 1242E positioned on either side of the hole 1242C. The first alignment features 1242E can be configured to align with a distal portion of the anchors 1237 to ensure proper placement of the skirt assembly 1238 relative to the inner and outer frames 1232, 1234. The first alignment features 1242E can include a plurality of holes, a plurality of dots, and/or other visual or tactile indicator.

The second alignment features 1242F can be positioned on at least one distal extension 1240C. In the illustrated configuration, each distal extension 1240C includes second alignment features 1242F along an edge of the distal extension 1240C. The second alignment features 1242F can be configured to be aligned with the inner skirt of the prosthetic valve assembly 1231 to ensure proper placement of the skirt assembly 1238 relative to the inner and outer frames 1232, 1234. The second alignment features 1242F can include a plurality of holes, a plurality of dots, and/or other visual or tactile indicator.

FIG. 17D illustrates the skirt assembly 1238 in a folded configuration with the distal extensions 1240C sewed together and the tapered sections 1242D sewed together. When the skirt assembly 1238 is folded, adjacent proximal extensions 1240A can overlap and/or adjacent distal extensions 1240C can overlap such that the adjacent proximal extensions 1240A and/or the adjacent distal extensions 1240C can be sewn together.

In some embodiments, a cloth material of the skirt assembly may be treated to soften an edge which may be roughened when laser cutting is applied. FIGS. 17E-1 and 17E-2 show softened edges of cloth material used for the skirt assembly of FIGS. 17A to 17D. The roughened edge can be softened by applying a soldering iron with heat within a threshold temperature to an edge of the integral piece of cloth material. For example, a soldering iron can be applied to melt the fibers of the cloth into one smooth melted edge 1238D. Alternatively, a z-axis feature of a laser to defocus the laser can be applied to create a thicker area of melted cloth that is smooth along the edge.

FIG. 17F shows a process of applying an interlocking stitch of the cloth material used for the skirt assembly of FIGS. 17A to 17D to eliminate knots. In the current method, a transcatheter heart valve is generally hand sewn using a suture, and therefore, there is typically a knot that acts as a speed bump for a delivery system to go over when the valve is crimped. In some implementations, an interlocking stitching technique can be applied to eliminate the knot. The interlocking stitch may use a woven structure of a suture itself to puncture and interlock within its own strands and can secure the suture without creating a bulky knot. In some implementations, with reference to FIG. 17F, a needle tip can be punctured within a center of the woven structure of the suture to form an interlocked structure, which can create a secure beginning or end point for the suture. The interlocking method may include sewing a needle through a force fiber (1), pulling a suture taut (2), sewing the needle through the force fiber again to create the interlock stitch on the opposite side (3), and finally pulling the suture taut again (4) to complete the interlock stitch.

### Additional Tether Retention Assembly Configuration

FIGS. 18A-18F show a configuration of a distal subassembly 1303. The distal subassembly 1303 can be similar to the configuration of the distal subassembly 703 illustrated in and described in relation to FIGS. 7A-7E. Reference numerals of the same or substantially the same features may share the same last two digits.

As shown in FIGS. 18A-18C, the distal tether retention component 1307 can be configured to retain the tether or suture 710. The tether or suture 710 can include a plurality of distal loops 1320. The distal tether retention component 1307 can be spaced from the proximal tether retention component 1306. For example, the distal subassembly 1303 can include a middle component 1312 between the proximal and distal tether retention components 1306, 1307. In some configurations, the middle component 1312 can include a tube. The middle component 1312 can be made of a metal material, such as stainless steel. In some configurations, the proximal tether retention component 1306 can have a diameter greater than a diameter of the middle component 1312 and/or the manifold cable 705. In some configurations, the distal tether retention component 1307 can have a diameter greater than the diameter of the middle component 1312 and/or the manifold cable 705.

As shown in FIG. 18A, the distal tether retention component 1307 can include a plurality of slots 1318 along the portion of the distal tether retention component 1307 that extends radially beyond the middle component 1312 and/or the manifold cable 705. The plurality of slots 1318 can include a length that extends along a longitudinal axis of the distal subassembly 1303. The illustrated configuration has nine slots 1318 in the distal tether retention component 1307. Other numbers of slots 1318 (e.g., two, four, five, six, seven, eight) may also be used in other configurations. The slots 1318 can be configured to receive portions of the tether or suture 710 and prevent the tether or suture 710 from being removed or uncoupled from the distal tether retention component 1307.

As shown in FIGS. 18B and 18C, the proximal tether retention component 1306 can include a plurality of slots 1314 along the portion of the proximal tether retention component 1306 that extends radially beyond the middle component 1312 and/or the manifold cable 705. The plurality of slots 1314 can include a length that extends along a longitudinal axis of the distal subassembly 1303. The number of slots 1314 of the proximal tether retention component 1306 may correspond with the number of slots 1318 of the distal tether retention component 1307. The illustrated configuration has nine slots 1314 in the proximal tether retention component 1306. Other numbers of slots 1314 (e.g., two, four, five, six, seven, eight) may also be used in other configurations. In some configurations, one or more of the slots 1314 can be configured to receive a distal loop 1320 of the tether or suture 710. In other configurations, one or more of the slots 1314 can be configured two or more distal loops 1320 of the tether or suture 710. In some configurations, the slots 1314 of the proximal tether retention component 1306 can align with the slots 1318 of the distal tether retention component 1307. In other configurations, the slots 1314 of the proximal tether retention component 1306 can be offset from the slots 1318 of the distal tether retention component 1307.

FIG. 18D illustrates the tether or suture 710 being secured to the distal subassembly 1303. As previously described, the slots 1314 can receive a distal loop 1320 of the tether or suture 710. A release (or locking) tether/suture 1322 can extend through the distal loops 1320, thus preventing the implant 30, 1230 from being released from the distal subassembly 1303 until ready. For example, a free end 1324 of the release tether/suture 1322 can be inserted through the distal loops of the tether or suture 710 to secure the tether or suture 710 to the implant 30, 1230.

FIGS. 18E and 18F illustrate the tether or suture 710 being removed from the distal subassembly 1303. The release tether/suture 1322 can be withdrawn so that a free end 1324 of the release tether/suture 1322 can pass through the distal loops 1320, thus releasing the implant 30, 1230 from the tethered attachment to the distal subassembly 1303. Multiple release (or locking) tethers/sutures 1322 may be used in some embodiments (e.g., one for each distal loop 1320 or one for multiple distal loops 1320).

FIGS. 19A and 19B illustrate another configuration of a proximal tether retention component 1406 and a middle component 1412 similar to the embodiments of the proximal tether retention component 706, 1306 and the middle component 1312 illustrated in and described in relation to FIGS. 7A-7E and 18A-18F. Reference numerals of the same or substantially the same features may share the same last two digits.

The plurality of slots 1414 of the proximal tether retention component 1406 can include three slots 1414. Each of the slots 1414 can be configured to receive one or more distal loops 1320 of the tether or suture 710 (not shown). In some configurations, as shown in FIG. 19A, the shaft extending between the middle component 1412 and the manifold cable 705 can include a plurality of apertures 1426. The apertures 1426 can be circumferentially spaced apart. As shown, the apertures 1426 can align with the slots 1414. In some configurations, the apertures 1426 can be at least partially offset from the slots 1414.

### Clocking or Implant Orientation Control

FIGS. 20A-20C illustrate a configuration of a handle 1514 similar to the embodiments of the handle 14 illustrated in and described in relation to FIGS. 1 and 11. The handle 1514 can be configured to rotate an implant 30, 1230 during delivery. For example, the implant 30, 1230 can be rotated to avoid certain anatomical structures, to enhance sealing of the implant 30, 1230, and/or to avoid erosion in certain anatomical areas (e.g., the aortic root in the atrium).

As shown, the handle 1514 can include a capsule knob 1505 (similar to the capsule knob 905 illustrated in and described in relation to FIGS. 9A and 9B), an orientation mechanism 1516 configured to rotate the implant 30, 1230 (not shown) during implantation, and a linear guide 1524. For example, the orientation mechanism 1516 can include an orientation knob 1516 extending from a side of the handle 1514 that can be rotated about a longitudinal axis of the orientation knob 1516. In some configurations, the handle 1514 can include a rotation mechanism 1518 coupled to the orientation knob 1516. When the orientation knob 1516 is rotated, the rotation mechanism 1518 can also rotate. In some configurations, the rotation mechanism 1518 can include a worm gear mechanism 1520 and an adapter 1522. The orientation knob 1516 can be coupled to the worm gear mechanism 1520 and be configured to rotate the worm gear mechanism 1520 when the orientation knob 1516 is rotated. The worm gear mechanism 1520 can be coupled to the linear guide 1524 such that the worm gear mechanism 1520 can rotate the linear guide 1524 when the orientation knob 1516 is rotated. The adapter 1522 can be coupled to the linear guide 1524 such that the linear guide 1524 can rotate the adapter 1522 when the linear guide 1524 is rotated. The adapter 1522 can be coupled to the outer proximal shaft 302 of the capsule assembly 306 (not shown). When the linear guide 1524 rotates the adapter 1522, the adapter 1522 can rotate the outer proximal shaft 302. In some configurations, the adapter 1522 can be configured to control linear motion of the outer proximal shaft 302 when the capsule knob 1505 is rotated.

In some configurations, the orientation knob 1516 can rotate the outer proximal shaft 302 of the capsule assembly 306. During delivery of the implant 30, 1230, the orientation knob 1516 can be actuated to rotate the outer proximal shaft 302 of the capsule subassembly 306 and the implant 30, 1230 within the capsule assembly 306 for positioning the implant 30, 1230 within the patient.

In some configurations, the orientation knob 1516 can include a plurality of indicators on an outer surface of the orientation knob 1516. The indicators on the orientation knob 1516 can correlate with the rotation of the implant 30, 1230. For example, the indicators can show a certain number of degrees that the implant 30, 1230 has been rotated. In some configurations, the orientation knob 1516 can be directly coupled to the outer proximal shaft 302 of the capsule assembly 306 such that rotating the orientation knob 1516 can directly rotate the outer proximal shaft 302. In some configurations, the orientation mechanism 1516 can be a lever configured to be pushed and/or pulled to rotate the implant 30, 1230 during delivery.

FIGS. 20D, 20E, 20F and 20G further illustrate an embodiment of an orientation mechanism of FIG. 20C connected to an outer lumen 20A within which an implant (e.g., implant 30, 1230) can be rotated. The detailed gear mechanism is described above in connection with FIGS. 20B and 20C, and therefore, the detailed description of the gear mechanism of the orientation mechanism is omitted here. By rotating the orientation mechanism or knob 1516, as shown in FIG. 20F to FIG. 20G, an implant 30, 1230 (not shown) can be rotated during implantation via the gear mechanism to position the implant to have a desired rotational orientation (e.g., to avoid potential for conduction disturbances caused by contact of a portion of the implant with certain tissue). As discussed previously, the gear mechanism can include a worm gear mechanism 1520 and a capsule adapter 1522. The orientation knob 1516 can be coupled to the worm gear mechanism 1520 and be configured to rotate the worm gear mechanism 1520 when the orientation knob 1516 is rotated. The worm gear mechanism 1520 can be coupled to a linear guide 1524 such that the worm gear mechanism 1520 can rotate the linear guide 1524 (and thus the outer sheath subassembly 20 and capsule subassembly 306 and the implant positioned therein) when the orientation knob 1516 is rotated. Rotation of the outer sheath subassembly 20 may passively cause rotation of other subassemblies and the implant due to being operably coupled to the outer sheath subassembly 20 but may not be rotated directly by rotation of the orientation knob 1516.

An implant (e.g., dual-frame valve prosthesis or replacement heart valve) may be pre-loaded with a desired orientation based on pre-procedural planning. For example, a predicted location of a bundle of His may be identified and a predicted amount of secondary flex believed to be required to implant the implant within a heart valve location may be determined. An orientation of the implant may be set during loading so as to avoid contact of an anchor or other implant portion with the bundle of His based on the determination. In addition, or alternatively, real-time clocking may be performed via the orientation mechanism 1516 based on direct or indirect fluoroscopy markers. Referring to FIGS. 20H to 201, which illustrate a virtual representation of implant 30, 1230 superimposed on images (e.g., fluoroscopic images) of the patient's inner body (e.g., heart anatomy) that have been taken before performing the rotation, the implant 30, 1230 can be positioned by rotating the orientation knob 1516, to avoid contact of one or more anchors 37 or other portions of the implant 30, 1230 with, for example, the bundle of His of the patient, represented by the marker 3000 superimposed on the image. The rotation (orientation) of the implant 30, 1230 can be performed intraprocedurally (e.g., by rotating from FIG. 20H to FIG. 20I) before deployment of the implant, to prevent (or reduce the likelihood of) the anchors 37 from contacting the bundle of His or other undesired tissue contact location based on the location of the marker 3000. That is, the orientation mechanism 1516 can be used not only during implantation as described with reference to FIGS. 23A-23C but also before delivery of the implant by marking a point 3000 to be avoided, e.g., the bundle of His of the patient, on the image taken before performing the delivery of the implant. With respect to indirect visualization, a relationship (e.g., angle offset Θ) between an anchor-free zone and a fluoroscopic indicator on the implant can be determined. Then, a marker 3000 identifying a location on the bundle of His can be marked and the angle offset Θ can be drawn on a pre-operative image (e.g., CT scan) of the patient's heart. The implant view can then be set to place the fluoroscopic plane orthogonal to the fluoroscopic indicator. The implant can then be loaded consistent to the determined angle offset Θ. Then, the clinician can bring the fluoroscopic indicator into a center of view in a fluoroscopic image to place the implant in a desired orientation without flipping to a direct fluoroscopic view. The fluoroscopic indicator may be an existing feature of the implant and not a separate indicator. In this instance, the loading step may not be necessary.

FIG. 21 illustrates another configuration of a handle 1614 similar to the embodiments of the handle 14, 1514 illustrated in and described in relation to FIGS. 1, 11, and 20A-20C. Reference numerals of the same or substantially the same features may share the same last two digits. The handle 1614 can be configured to rotate an implant 30, 1230 during delivery. The orientation knob 1616 can extend along a longitudinal axis of the handle 1614 and be configured to rotate about the longitudinal axis of the handle 1614. The orientation knob 1616 can be configured to rotate the outer proximal shaft 302 and the implant 30, 1230 when the orientation knob 1616 is rotated.

FIGS. 22-23C illustrate an implant 30 delivered to a heart. As shown, the heart may include a hot spot 2000. The hot spot 2000 can be within the ventricular septum of the heart near the aortic valve that includes conduction fibers (e.g., right and/or left branches of the bundle of His). When the implant 30 is delivered to the tricuspid valve of the heart, the anchors 37 of the implant 30 may contact the conduction fibers within the hot spot 2000. If the anchors 37 of the implant 30 contact the conduction fibers, this can cause an atrioventricular block (“AV block”) within the tricuspid valve. Advantageously, any of the orientation knobs 1516, 1616 described herein can be used to rotate, or clock, the implant 30 during delivery so that the anchors 37 of the implant 30 do not contact the conduction fibers. For example, the clocking of the implant may advantageously cause the anchors 37 to avoid the main fibrous bundle running along the right ventricle septum near the aortic valve. In addition, clocking functionality may facilitate use of asymmetric implant designs that may offer additional benefits, such as enhanced sealing capability or avoidance of erosion in key areas, such as the aortic root in the atrium. Although the implant 30 is shown and described, other implants (e.g., implant 1230 or other implants described herein) can also be delivered or “clocked” as described herein.

### Additional Statements and Terminology

From the foregoing description, it will be appreciated that an inventive product and approaches for implant delivery systems are disclosed. While several components, techniques and aspects have been described with a certain degree of particularity, it is manifest that many changes can be made in the specific designs, constructions and methodology herein above described without departing from the spirit and scope of this disclosure.

The section headings used herein are merely provided to enhance readability and are not intended to limit the scope of the embodiments disclosed in a particular section to the features or elements disclosed in that section. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination. In some embodiments, the delivery system or delivery device comprises various features that are present as single features (as opposed to multiple features). For example, in one embodiment, the delivery system includes a single delivery device with a single implant. Multiple features or components are provided in alternate embodiments.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Spatially relative terms, such as “proximal”, “distal”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

In some configurations, the delivery system comprises one or more of the following: means for introducing the delivery device, means for stabilizing the delivery device, means for steering the delivery device, means for releasing the implant from the delivery device, etc.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A delivery system for delivering an expandable implant to a body location, the delivery system comprising:
an outer sheath subassembly comprising an outer proximal shaft having an outer lumen and a proximal end and a distal end, wherein the outer sheath subassembly comprises a capsule subassembly configured to retain the expandable implant in a compressed configuration;
a rail subassembly located within the outer lumen, the rail subassembly comprising a rail shaft having a proximal end and a distal end, wherein the rail subassembly comprises one or more pull wires attached to the rail shaft configured to provide an axial force on the rail shaft to steer the rail subassembly;
a mid-shaft subassembly within a lumen of the rail subassembly, the mid-shaft subassembly comprising a hypotube having a lumen and a proximal end and a distal end, wherein the mid-shaft subassembly comprises a pusher configured to radially restrain at least a proximal-most portion of the expandable implant; and
a proximal handle comprising a capsule knob operably coupled to the outer sheath subassembly and one or more flex knobs operably coupled to the one or more pull wires, wherein rotation of the capsule knob causes translation of the outer sheath subassembly, and wherein rotation of the one or more flex knobs causes bending of the rail shaft.

2. The delivery system of claim 1, wherein a distal tip of the capsule subassembly comprises a plurality of lobes configured to facilitate staggered recapture of distal anchors of the expandable implant as the outer sheath subassembly is translated distally to recapture the expandable implant after initial deployment.

3. The delivery system of claim 1, wherein the pusher comprises a flat distal contact surface without a lip or cup portion.

4. The delivery system of claim 1, wherein the handle further comprises an orientation knob configured to rotate an orientation of the expandable implant.

5. The delivery system of claim 4, wherein the orientation knob is operably coupled to a worm gear that is operably coupled to an adapter and linear guide of the outer sheath subassembly positioned within the handle such that rotation of the orientation knob causes rotation of the linear guide of the outer sheath subassembly and thus rotation of the implant retained within the capsule subassembly of the outer sheath subassembly.

6. The delivery system of claim 1, further comprising the expandable implant, wherein the expandable implant comprises a frame having an inflow end and an outflow end, wherein the inflow end comprises an axial inflow strut having an eyelet configured to receive a tether loop to facilitate releasable attachment of the expandable implant to the delivery system, wherein a radius of curvature of a proximal upper edge of a proximal tip of the axial inflow strut is less than a height from the proximal tip of the axial inflow strut to a distal-most edge of the eyelet.

7. The delivery system of claim 6, wherein the eyelet has a semi-circular shape, an oval shape, or a bean shape.

8. A delivery system for delivering an expandable implant to a body location, the delivery system comprising:
a shaft assembly comprising a proximal end portion and a distal end portion, wherein the proximal end portion of the shaft assembly comprises a handle including at least one actuator;
a plurality of suture portions,
wherein a first end of each of the plurality of suture portions is permanently coupled to the distal end portion of the shaft assembly,
wherein a second end of each of the plurality of suture portions is removably coupled to at least one retention member of the distal end portion of the shaft assembly after being inserted through a coupling member of an implant,
wherein actuation of the at least one actuator causes the second end of each of the plurality of suture portions to be decoupled from the at least one retention member of the distal end portion of the shaft assembly.

9. The delivery system of claim 8, wherein the at least one retention member comprises a plurality of retention members spaced circumferentially around the distal end portion of the shaft assembly.

10. The delivery system of claim 8, wherein the second end of each of the plurality of suture portions comprises a suture loop that is configured to be looped onto the at least one retention member.

11. The delivery system of claim 8, wherein the at least one actuator comprises a rotatable knob.

12. The delivery system of claim 8, wherein the plurality of suture portions are formed from a single continuous suture strand.

13. The delivery system of claim 8, wherein the shaft assembly comprises an inner manifold shaft and an outer release shaft, wherein the outer release shaft is coaxial with the inner release shaft.

14. The delivery system of claim 13, wherein the first end of each of the plurality of suture portions is permanently coupled to the distal end portion of the inner manifold shaft, wherein the second end of each of the plurality of suture portions is removably coupled to at least one retention member of the distal end portion of the inner manifold shaft, wherein actuation of the at least one actuator causes translation of the inner manifold shaft with respect to the outer release shaft, and wherein translation of the inner manifold shaft with respect to the outer release shaft causes at least one structure of the distal end portion of the outer release shaft to decouple the second end of each of the plurality of suture portions from the at least one retention member of the distal end portion of the outer release shaft.

15. The delivery system of claim 14, wherein the at least one structure comprises a plurality of windows, and wherein the second end of each of the plurality of suture portions is inserted through one of the plurality of windows prior to being removably coupled to the at least one retention member of the distal end portion of the inner manifold shaft.

16. The delivery system of claim 8, wherein the handle further comprises an orientation knob configured to rotate an orientation of the expandable implant.

17. The delivery system of claim 8, wherein the at least one retention member comprises three retention members.

18. The delivery system of claim 8, wherein the at least one retention member comprises a release suture.

19. A delivery system for delivering an expandable implant to a body location, the delivery system comprising:
an outer shaft comprising a proximal end and a distal end;
an inner manifold shaft comprising a proximal end and a distal end, wherein the manifold shaft is coaxially positioned within the outer shaft, wherein the inner manifold shaft comprises a distal tether retention component at the distal end of the manifold shaft;
nine suture loops, wherein each of the nine suture loops comprise a first end opposite a loop end, wherein the first end is non-removably coupled to the distal tether retention component; and
a release suture configured to releasably secure the nine suture loops to the inner manifold shaft;
wherein the loop end of each of the nine suture loops is looped through a respective eyelet positioned at a proximal end of a replacement heart valve,
wherein each loop end of the nine suture loops is configured to receive the release suture, and
wherein removing the release suture from the loop end of the nine suture loops allows the suture loops to be released from the respective eyelets, thereby allowing the replacement heart valve to be decoupled from the delivery device.

20. The delivery system of claim 19, wherein the nine suture loops are formed from a single continuous strand.
